(19) United States
(12) Patent Application Publication
(10) Pub. No.: US 2023/0368465 A1
(43) Pub. Date: Nov. 16, 2023

(54) METHODS AND APPARATUS FOR THREE-DIMENSIONAL RECONSTRUCTION

(71) Applicant: JointVue LLC, Knoxville, TN (US)

(72) Inventors: Mohamed R. Mahfouz, Knoxville, TN (US); Manh Duc Ta, Knoxville, TN (US)

(73) Assignee: JointVue LLC, Knoxville, TN (US)

(21) Appl. No.: 18/316,276

(22) Filed: May 12, 2023

Related U.S. Application Data
(60) Provisional application No. 63/364,656, filed on May 13, 2022.

(51) Int. Cl. G06T 17/00; A61B 34/10; G06T 7/60; G06T 19/20; G06T 7/33

(52) U.S. Cl. CPC G06T 17/00; A61B 34/10; G06T 7/60; G06T 19/20; G06T 7/344; A61B 2034/105; A61B 2034/107; G06T 2210/41; G06T 2219/2004; G06T 2210/56; G06T 2207/10132; G06T 2207/30012; G06T 2207/10028; G06T 2207/10116

(57) ABSTRACT

Methods of generating three-dimensional models of musculoskeletal systems, and three-dimensional bone and soft tissue model reconstruction, and associated apparatus, are disclosed. An example method of generating a virtual 3-D patient-specific bone model may include obtaining a preliminary 3-D bone model of a first bone; obtaining a supplemental image of the first bone; registering the preliminary 3-D bone model of the first bone with the supplemental image of the first bone; extracting geometric information about the first bone from the supplemental image of the first bone; and/or generating a virtual 3-D patient-specific bone model of the first bone by refining the preliminary 3-D bone model of the first bone using the geometric information about the first bone from the supplemental image of the first bone.

### CROSS-REFERENCE TO RELATED APPLICATIONS

This application claims the benefit of U.S. Provisional Patent Application No. 63/364,656, titled “METHODS AND APPARATUS FOR THREE-DIMENSIONAL RECONSTRUCTION USING MULTIPLE IMAGING MODALITIES,” filed May 13, 2022, which is incorporated by reference in its entirety.

### INTRODUCTION

The present disclosure relates generally to methods of generating three-dimensional virtual models of musculoskeletal systems and, more particularly, to three-dimensional bone and soft tissue model reconstruction, and associated apparatus.

The present disclosure contemplates that three-dimensional (“3-D”) models of anatomical structures, such as musculoskeletal systems (e.g., bones, ligaments, tendons, and/or cartilage), may be used in connection with diagnosis and/or treatment involving such musculoskeletal systems. For example, 3-D bone models may be used in connection with orthopedic surgery, such as for preoperative planning, intraoperative surgical navigation, intraoperative bone preparation, and/or postoperative assessment.

The present disclosure contemplates that various imaging modalities which may be used in connection with anatomical structures may be associated with certain potential advantages and/or potential disadvantages. For example, in the field of orthopedics, ultrasound imaging may facilitate highly accurate 3-D surface mapping and may not expose the patient or nearby persons to ionizing radiation. However, ultrasound may generally be limited to imaging the exterior features of bones. More specifically, ultrasound may be limited in its ability to image certain anatomical structures, such as internal features of bones and/or external features of bones that are occluded by other bones. As another example, X-ray imaging and/or fluoroscopic imaging may allow visualization of internal features of bones and/or portions of bones that are occluded by other bones. However, these modalities may expose the patient and nearby persons to ionizing radiation. Additionally, most common X-ray and fluoroscopic imaging techniques provide only two-dimensional imaging.

Accordingly, there is a need for improved methods and apparatuses associated with 3-D imaging of musculoskeletal features.

It is an aspect of the present disclosure to provide a method of generating a virtual 3-D patient-specific bone model, including obtaining a preliminary virtual 3-D bone model of a first bone; obtaining a supplemental image of the first bone; registering the preliminary virtual 3-D bone model of the first bone with the supplemental image of the first bone; extracting geometric information about the first bone from the supplemental image of the first bone; and/or generating a refined virtual 3-D patient-specific bone model of the first bone by refining the preliminary virtual 3-D bone model of the first bone using the geometric information about the first bone from the supplemental image of the first bone.

In a detailed embodiment, obtaining the preliminary 3-D bone model may include obtaining a point cloud of the first bone and reconstructing the preliminary 3-D bone model by morphing a generalized 3-D bone model using the point cloud of the first bone. Obtaining the point cloud of the first bone may utilize a first imaging modality. Obtaining the supplemental image of the first bone may utilize a second imaging modality. The first imaging modality may be different than the second imaging modality. The first imaging modality may include ultrasound. The second imaging modality may include 2-D X-ray. The second imaging modality may include 2-D X-ray.

In a detailed embodiment, the supplemental image of the first bone may include at least one portion of the first bone that was not included in the point cloud of the first bone. Obtaining the point cloud of the first bone may include performing an ultrasound scan of the first bone. Obtaining the supplemental image of the first bone may include obtaining a 2-D X-ray of the first bone. The 2-D X-ray of the first bone may include at least one portion of the first bone that was not available from the ultrasound scan of the first bone. The at least one portion of the first bone that was not available from the ultrasound scan of the first bone may have been at least partially occluded from ultrasound scanning by an anatomical structure.

In a detailed embodiment, the at least one portion of the first bone that was not available from the ultrasound scan of the first bone may include an internal structure of the first bone. The occluded internal structure of the first bone may include a medullary canal. The first bone may include a femur. The medullary canal may include the femoral medullary canal.

In a detailed embodiment, the at least one portion of the first bone that was not visible on the ultrasound scan of the first bone may include an external structure of the first bone. The external structure of the first bone may have been at least partially occluded from ultrasound scanning by a second bone. One of the first bone and the second bone may include a femoral head and the other of the first bone and the second bone may include an acetabular cup.

In a detailed embodiment, the external structure of the first bone that was occluded from ultrasound scanning by the second bone may include a soft tissue. The soft tissue may include cartilage. The cartilage may include hip articular cartilage. The cartilage may include knee articular cartilage. The cartilage may include shoulder articular cartilage.

In a detailed embodiment, each of the first bone and the second bone may include one or more of a pelvis, a femur, a tibia, a patella, a scapula, and a humerus.

In a detailed embodiment, the first bone may include one or more of a pelvis, a femur, a tibia, a patella, a scapula, and a humerus.

In a detailed embodiment, registering the preliminary 3-D bone model of the first bone with the supplemental image of the first bone may include solving for a pose of the preliminary 3-D bone model which produces a 2-D projection corresponding to a projection of the supplemental image.

In a detailed embodiment, obtaining a supplemental image of the first bone may include obtaining a plurality of supplemental images of the first bone. Registering the preliminary virtual 3-D bone model of the first bone with the supplemental image of the first bone may include registering the preliminary virtual 3-D bone model of the first bone with the plurality of supplemental images of the first bone. Extracting geometric information about the first bone from the supplemental images of the first bone may include extracting geometric information about the first bone from the plurality of supplemental images of the first bone. Refining the preliminary virtual 3-D bone model of the first bone using the geometric information about the first bone from the supplemental image of the first bone may include refining the preliminary virtual 3-D bone model of the first bone using the geometric information about the first bone from the plurality of supplemental images of the first bone.

In a detailed embodiment, the method may include obtaining a preliminary virtual 3-D bone model of a second bone; obtaining a supplemental image of the second bone; registering the preliminary virtual 3-D bone model of the second bone with the supplemental image of the second bone; extracting geometric information about the second bone from the supplemental image of the second bone; and/or generating a refined virtual 3-D patient-specific bone model of the second bone by refining the preliminary virtual 3-D bone model of the second bone using the geometric information about the second bone from the supplemental image of the second bone.

In a detailed embodiment, obtaining the point cloud of the second bone may include performing an ultrasound scan of the second bone. Obtaining the supplemental image of the second bone may include obtaining a 2-D X-ray of the second bone. The 2-D X-ray of the second bone may include at least one portion of the second bone that was not visible on the ultrasound scan of the second bone.

In a detailed embodiment, extracting geometric information from the supplemental image of the first bone may include extracting at least one of a length dimension, an angular dimension, or a curvature of the first bone from the supplemental image of the first bone.

In a detailed embodiment, a method of preoperatively sizing an orthopedic implant may include generating the refined virtual 3-D patient-specific bone model according to the method described above; and/or sizing an orthopedic implant using the refined virtual 3-D patient-specific bone model.

In a detailed embodiment, an apparatus may be configured to perform the method described above. In a detailed embodiment, a memory may include instructions that, when executed by a processor, cause the processor to perform the method described above.

It is an aspect of the present disclosure to provide a method of generating a virtual 3-D patient-specific bone model, including obtaining ultrasound data pertaining to an exterior surface of a first bone; obtaining X-ray data pertaining to at least one of an internal feature of the first bone and/or an occluded feature of the first bone; and/or generating a 3-D patient-specific bone model of the first bone using the ultrasound data and the X-ray data, the 3-D patient-specific bone model representing the exterior surface of the first bone and the at least one of the internal feature of the first bone and/or the occluded feature of the first bone.

In a detailed embodiment, obtaining the ultrasound data pertaining to the exterior surface of the first bone may include obtaining an ultrasound point cloud of the exterior surface of the first bone and generating a preliminary 3-D bone model of the first bone. Generating the 3-D patient-specific bone model of the first bone may include refining the preliminary 3-D bone model of the first bone using the X-ray data.

In a detailed embodiment, an apparatus may be configured to perform the method described above. In a detailed embodiment, a memory may include instructions that, when executed by a processor, cause the processor to perform the method described above.

It is an aspect of the present disclosure to provide a method of determining a spine-pelvis tilt, including obtaining a virtual 3-D model of a pelvis; obtaining a first ultrasound point cloud of the pelvis and a first ultrasound point cloud of a lumbar spine with the pelvis and the lumbar spine in a first functional position; registering the virtual 3-D model of the pelvis to the first point cloud of the pelvis; and/or determining a first spine-pelvis tilt in the first functional position using a first relative angle of the first point cloud of the lumbar spine to the 3-D model of the pelvis.

In a detailed embodiment, the method may include positioning at least one of the pelvis or the lumbar spine into the first functional position.

In a detailed embodiment, the method may include obtaining a second ultrasound point cloud of the pelvis and a second ultrasound point cloud of the lumbar spine with the pelvis and the spine in a second functional position; registering the virtual 3-D model of the pelvis to the second point cloud of the pelvis; and/or determining a second spine-pelvis tilt in the second functional position using a second relative angle of the second point cloud of the lumbar spine to the 3-D model of the pelvis.

In a detailed embodiment, the method may include positioning at least one of the pelvis or the lumbar spine into the second functional position.

In a detailed embodiment, the method may include obtaining a third ultrasound point cloud of the pelvis and a third ultrasound point cloud of the lumbar spine with the pelvis and the lumbar spine in a third functional position; registering the virtual 3-D model of the pelvis to the third point cloud of the pelvis; and/or determining a third spine-pelvis tilt in the third functional position using a third relative angle of the third point cloud of the lumbar spine to the 3-D model of the pelvis.

In a detailed embodiment, the method may include positioning at least one of the pelvis or the lumbar spine into the third functional position.

In a detailed embodiment, each of first functional position, the second functional position, and the third functional position may include one of a sitting position, a standing position, and/or a supine position.

In a detailed embodiment, obtaining the virtual 3-D model of the pelvis may include generating the virtual 3-D model of the pelvis using ultrasound.

In a detailed embodiment, obtaining the first ultrasound point cloud of the pelvis and the first ultrasound point cloud of the lumbar spine in the first functional position may include obtaining a sparse ultrasound point cloud of the pelvis and a sparse ultrasound point cloud of the lumbar spine.

In a detailed embodiment, at least one of the first ultrasound point cloud of the pelvis and the first ultrasound point cloud of the lumbar spine with the pelvis and the lumbar spine in the first functional position may include additional points pertaining to a femur. The method may include determining at least one of a femoral version, an acetabular version, or a combined version. Determining the at least one of the femoral version, the acetabular version, and/or the combined version may include identifying an transepicondylar axis or a posterior condylar axis of the femur to determine a femoral version angle reference axis.

In a detailed embodiment, the method may include obtaining information pertaining to a leg length by obtaining data from at least one X-ray taken with the subject in a standing position.

In a detailed embodiment, an apparatus may be configured to perform the method described above. In a detailed embodiment, a memory may include instructions that, when executed by a processor, cause the processor to perform the method described above.

It is an aspect of the present disclosure to provide a method of generating a virtual 3-D patient-specific model of a ligament, including obtaining a virtual 3-D patient-specific bone model of a joint; detecting at least one ligament loci on the virtual 3-D patient-specific bone model; obtaining ultrasound data pertaining to a ligament associated with the at least one ligament loci by scanning, using ultrasound, the ligament; and/or reconstructing a virtual 3-D model of the ligament using the ultrasound data.

In a detailed embodiment, obtaining the ultrasound data pertaining to the ligament may be performed at a plurality of joint angles of the joint across the joint's range of motion.

In a detailed embodiment, obtaining the virtual 3-D patient-specific bone model of the joint may include reconstructing the joint using ultrasound. Reconstructing the joint using ultrasound may include obtaining at least one point cloud associated with one or more bones of the joint.

In a detailed embodiment, detecting the at least one ligament loci on the patient-specific virtual 3-D bone model may include determining at least one insertion location of the ligament.

In a detailed embodiment, scanning, using ultrasound, the ligament may include providing automated guidance information. Providing the automated guidance information may include providing a display comprising a current position of an ultrasound probe relative to one or more anatomical structures. Providing the automated guidance information may include providing a display comprising an indication of a desired location or direction of scanning.

Providing the automated guidance information may include providing a display comprising an A-mode or B-mode ultrasound image.

In a detailed embodiment, the joint may include a knee. The ligament may include a medial collateral ligament.

In a detailed embodiment, the joint may include a knee. The ligament may include a lateral collateral ligament.

In a detailed embodiment, an apparatus may be configured to perform the method described above. In a detailed embodiment, a memory may include instructions that, when executed by a processor, cause the processor to perform the method described above.

It is an aspect of the present disclosure to provide a method of generating a virtual 3-D patient-specific anatomical model, including obtaining a preliminary virtual 3-D anatomy model of a first anatomy; obtaining a supplemental image of the first anatomy; registering the preliminary virtual 3-D anatomy model of the first anatomy with the supplemental image of the first anatomy; extracting geometric information about the first anatomy from the supplemental image of the first anatomy; and/or generating a refined virtual 3-D patient-specific anatomy model of the first anatomy by refining the preliminary virtual 3-D anatomy model of the first anatomy using the geometric information about the first anatomy from the supplemental image of the first anatomy.

In a detailed embodiment, obtaining the preliminary 3-D anatomy model may include obtaining a point cloud of the first anatomy and reconstructing the preliminary 3-D anatomy model by morphing a generalized 3-D anatomy model using the point cloud of the first anatomy. Obtaining the point cloud of the first anatomy may utilize a first imaging modality. Obtaining the supplemental image of the first anatomy may utilize a second imaging modality. The first imaging modality may be different than the second imaging modality.

In a detailed embodiment, the first imaging modality may include ultrasound. The second imaging modality may include 2-D X-ray. The second imaging modality may include 2-D X-ray.

In a detailed embodiment, the supplemental image of the first anatomy may include at least one portion of the first anatomy that was not included in the point cloud of the first anatomy. Obtaining the point cloud of the first anatomy may include performing an ultrasound scan of the first anatomy. Obtaining the supplemental image of the first anatomy may include obtaining a 2-D X-ray of the first anatomy. The 2-D X-ray of the first anatomy may include at least one portion of the first anatomy that was not available from the ultrasound scan of the first anatomy. The at least one portion of the first anatomy that was not available from the ultrasound scan of the first anatomy may have been at least partially occluded from ultrasound scanning by an anatomical structure.

In a detailed embodiment, the at least one portion of the first anatomy that was not available from the ultrasound scan of the first anatomy may include an internal structure of the first anatomy. The occluded internal structure of the first anatomy may include a medullary canal. The first anatomy may include a femur. The medullary canal may include the femoral medullary canal.

In a detailed embodiment, the at least one portion of the first anatomy that was not visible on the ultrasound scan of the first anatomy may include an external structure of the first anatomy. The external structure of the first anatomy may have been at least partially occluded from ultrasound scanning by a second anatomy. One of the first anatomy and the second anatomy may include a femoral head and the other of the first anatomy and the second anatomy may include an acetabular cup.

In a detailed embodiment, the external structure of the first anatomy that was occluded from ultrasound scanning by the second anatomy may include a soft tissue. The soft tissue may include cartilage. The cartilage may include hip articular cartilage. The cartilage may include knee articular cartilage. The cartilage may include shoulder articular cartilage.

In a detailed embodiment, each of the first anatomy and the second anatomy may include one or more of a pelvis, a femur, a tibia, a patella, a scapula, and/or a humerus.

In a detailed embodiment, the first anatomy may include one or more of a pelvis, a femur, a tibia, a patella, a scapula, and/or a humerus.

In a detailed embodiment, registering the preliminary 3-D anatomy model of the first anatomy with the supplemental image of the first anatomy may include solving for a pose of the preliminary 3-D anatomy model which produces a 2-D projection corresponding to a projection of the supplemental image.

In a detailed embodiment, obtaining a supplemental image of the first anatomy may include obtaining a plurality of supplemental images of the first anatomy. Registering the preliminary virtual 3-D anatomy model of the first anatomy with the supplemental image of the first anatomy may include registering the preliminary virtual 3-D anatomy model of the first anatomy with the plurality of supplemental images of the first anatomy. Extracting geometric information about the first anatomy from the supplemental images of the first anatomy may include extracting geometric information about the first anatomy from the plurality of supplemental images of the first anatomy. Refining the preliminary virtual 3-D anatomy model of the first anatomy using the geometric information about the first anatomy from the supplemental image of the first anatomy may include refining the preliminary virtual 3-D anatomy model of the first anatomy using the geometric information about the first anatomy from the plurality of supplemental images of the first anatomy.

In a detailed embodiment, the method may include obtaining a preliminary virtual 3-D anatomy model of a second anatomy; obtaining a supplemental image of the second anatomy; registering the preliminary virtual 3-D anatomy model of the second anatomy with the supplemental image of the second anatomy; extracting geometric information about the second anatomy from the supplemental image of the second anatomy; and/or generating a refined virtual 3-D patient-specific anatomy model of the second anatomy by refining the preliminary virtual 3-D anatomy model of the second anatomy using the geometric information about the second anatomy from the supplemental image of the second anatomy. Obtaining the point cloud of the second anatomy may include performing an ultrasound scan of the second anatomy. Obtaining the supplemental image of the second anatomy may include obtaining a 2-D X-ray of the second anatomy. The 2-D X-ray of the second anatomy may include at least one portion of the second anatomy that was not visible on the ultrasound scan of the second anatomy.

In a detailed embodiment, extracting geometric information from the supplemental image of the first anatomy may include extracting at least one of a length dimension, an angular dimension, or a curvature of the first anatomy from the supplemental image of the first anatomy.

In a detailed embodiment, a method of preoperatively sizing an orthopedic implant may include generating the refined virtual 3-D patient-specific anatomy model according to the method described above; and/or sizing an orthopedic implant using the refined virtual 3-D patient-specific anatomy model.

In a detailed embodiment, an apparatus may be configured to perform the method described above. In a detailed embodiment, a memory may include instructions that, when executed by a processor, cause the processor to perform the method described above.

In a detailed embodiment, the first anatomy may include a first bone.

It is an aspect of the present disclosure to provide any method, process, device, apparatus, or system associated with any aspect or embodiment described above, or as described herein. It is an aspect of the present disclosure to provide any combination of any elements of any of the preceding aspects or embodiments, or as described herein.

### BRIEF DESCRIPTION OF THE DRAWINGS

The accompanying drawings, which are incorporated in and constitute a part of this specification, illustrate embodiments of the present disclosure and, together with the detailed description of the exemplary embodiments given below, serve to explain the principles of the present disclosure.

FIG. 1 is a perspective view of an ultrasound instrument in accordance with one embodiment of the present invention.

FIG. 2 is a perspective view of a hybrid probe comprising an ultrasound probe and an optical marker, in accordance with one embodiment of the present invention.

FIG. 2A is a side elevational view of a position sensor for use with the optical marker of the hybrid probe.

FIG. 3 is a diagrammatic view of a computer system suitable for generating a 3-D patient-specific bone model from A-mode ultrasound RF signals in accordance with one embodiment of the present invention.

FIG. 4 is a flow chart illustrating one exemplary method of calibrating the optical system and generating a transformation between a local frame and a world frame.

FIGS. 5A-5C are diagrammatic views of a knee joint, showing the anterior, the medial, and the posterior portions, respectively.

FIGS. 6A-6F are fluoroscopic images of the knee joint in a plurality of degrees of flexion.

FIG. 7 is a flow chart illustrating one exemplary method of acquiring A-mode ultrasound RF signal and generating the 3-D patient-specific bone model.

FIG. 8 is a diagrammatic view of the method of acquiring A-mode ultrasound RF signals in accordance with FIG. 7.

FIG. 9 is a B-mode ultrasound image of a knee joint, which may be generated from the A-mode ultrasound RF signal.

FIG. 10A is an example of a raw RF signal as acquired by one transducer of the transducer array of an ultrasound probe.

FIG. 10B is an ultrasound frame illustrating select ones of the RF signals overlaid the B-mode ultrasound image of FIG. 9.

FIG. 10C is the ultrasound frame of FIG. 10B with a bone echo contour identified.

FIG. 10D is a 3-D rendering of the RF signals acquired in a data frame, which is shown in the B-mode image format in FIG. 10C.

FIG. 10E is another 3-D rendering of an ultrasound frame with select ones of the RF signals delineated.

FIG. 11 is a flow chart illustrating one exemplary method of identifying and extracting the bone echo from the A-mode ultrasound RF signal.

FIG. 12A is a 3-D rendering of an ultrasound frame after envelope detection.

FIGS. 12B-12E respectively illustrate four exemplary envelopes of the sampled A-mode ultrasound RF signal, with the echoes identified in each envelope.

FIGS. 13A and 13D are B-mode ultrasound frames calculated from exemplary A-mode ultrasound RF signals.

FIGS. 13B and 13E are ultrasound frames corresponding to FIGS. 13A and 13-D, respectively, with a bone contour identified before noise removal and overlain on the B-mode image.

FIGS. 13C and 13F are plots of the local standard deviation of the bone contours of FIGS. 13B and 13E, respectively.

FIGS. 14A and 14D are ultrasound frames illustrating exemplary B-mode images constructed from A-mode ultrasound RF signals, and in which no bone tissue was scanned.

FIGS. 14B and 14E are ultrasound frames corresponding to FIGS. 14A and 14D, respectively, with the noisy false bone contours shown.

FIGS. 14C and 14F are plots of the local standard deviation of the last echoes of FIGS. 14B and 14E, respectively.

FIG. 15 is a flow chart illustrating one exemplary method of generating a bone point cloud from the isolated bone contours.

FIGS. 16A, 16C, 17A, and 17C are exemplary bone point clouds, generated in accordance with one embodiment of the present invention.

FIGS. 16B, 16D, 17B, and 17D are examples in which the bone point clouds of FIGS. 16A, 16C, 17A, and 17C, respectively, are aligned to a bone model.

FIG. 18 is a flow chart illustrating one exemplary method of generating a statistical atlas of bone models.

FIG. 19 is a flow chart illustrating one exemplary method of optimizing a bone model to the bone point cloud.

FIG. 20 is a diagrammatic view of a medical imaging system including an ultrasound machine, electromagnetic tracking system, and a computer that operate cooperatively to provide real-time 3-D images to the attending physician.

FIG. 21 is a flow chart illustrating a method in accordance with an alternative embodiment of the invention by which the imaging system in FIG. 20 generates a real-time 3-D bone model.

FIG. 22 is a graphical view illustrating an ultrasound signal that is swept in frequency.

FIGS. 23A and 23B are graphical views illustrating an RF signal, a signal envelope generated from the RF signal, and a plurality of amplitude peaks identified in the signal envelope using a linear Gaussian filter.

FIGS. 24A-24D are graphical views illustrating an RF signal, a signal envelope generated from the RF signal, and a plurality of amplitude peaks identified in the signal envelope using a non-linear, non-Gaussian filter.

FIG. 25 is a graphical view illustrating one method by which a contour line is derived from a plurality of ultrasound scan line signal envelopes.

FIG. 26 is a graphical view illustrating a contour generated from a plurality of ultrasound scan line envelopes using first peak detection, and a contour generated from the plurality of scan line envelopes using a Bayesian smoothing filter.

FIG. 27 is a 3-D view of an ultrasound frame after envelope detection, and a corresponding registered point cloud for an imaged joint.

FIG. 28 is a flow diagram of an example method of generating a virtual 3-D model of an anatomical structure using multiple imaging modalities.

FIG. 29A is an isometric view of an example ultrasound point cloud of a femur.

FIG. 29B is an isometric view of an example ultrasound point cloud of pelvis.

FIG. 30A is an isometric view of the point clouds of FIGS. 29A and 29B arranged as obtained by scanning a patient's hip joint.

FIG. 30B is an isometric view of the point clouds of FIGS. 29A and 29B overlaid on a preliminary 3-D model.

FIG. 31 is an example supplemental image comprising a 2-D X-ray.

FIG. 32 illustrates a preliminary 3-D model registered with the supplemental image.

FIG. 33 illustrates a refined 3-D model overlain with the supplemental image.

FIG. 34 illustrates an example display facilitating anatomical measurements using the refined 3-D model.

FIG. 35 is a flow diagram illustrating an example method of determining spine-pelvis tilt.

FIG. 36 is a flow diagram of an example method of generating a virtual 3-D model of an anatomical structure including at least one ligament or other soft tissue.

FIG. 37 is an example display shown during an ultrasound scan of a femur.

FIG. 38 is an example display shown during an ultrasound scan of a lateral aspect of a knee.

FIG. 39 is an example display shown during an ultrasound scan of a lateral aspect of a knee.

FIG. 40 is an example display shown during an ultrasound scan of a medial aspect of a knee.

### DETAILED DESCRIPTION

The present disclosure includes, among other things, methods and apparatuses associated with creation of virtual models of anatomical structures, such as generation of 3-D models of musculoskeletal features. Some example embodiments according to at least some aspects of the present disclosure are described and illustrated below to encompass devices, methods, and techniques relating to generation of virtual musculoskeletal models using multiple imaging modalities, such as ultrasound and X-ray imaging. Of course, it will be apparent to those of ordinary skill in the art that the embodiments discussed below are examples and may be reconfigured and combined without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure.

Some example embodiments according to at least some aspects of the present disclosure may utilize ultrasound imaging in connection with reconstruction of 3-D models of anatomical structures. Accordingly, the following section provides a description of exemplary methods and apparatus for reconstructing 3-D models of joints (e.g., bones and/or soft tissues) using ultrasound.

3-D Reconstruction of Joints Using Ultrasound

The reconstruction of a 3-D model for joint, such as the articulating bones of a knee, is a key component of computer-aided joint surgery systems. The existence of a pre-operatively acquired model enables the surgeon to pre-plan a surgery by choosing the proper implant size, providing the femoral and tibial cutting planes in the case of knee surgery, and evaluating the fit of the chosen implant. The conventional method of generating the 3-D model is segmentation of computed tomography (“CT”), or magnetic resonance imaging (“MRI”) scans, which are the conventional imaging modalities for creating 3-D patient-specific bone models. The segmentation methods used are either manual, semi-automated, or fully automated. Although these methods produce highly accurate models, CT and MRI have inherent drawbacks, i.e., both are fairly expensive procedures (especially for the MRI), and CT exposes the patient to ionizing radiation.

One alternative method of forming 3-D patient-specific models is the use of previously acquired X-ray images as a priori information to guide the morphing of a generalized bone model whose projection matches the X-ray images. Several X-ray based model reconstruction methodologies have been developed for the femur (including, specifically, the proximal and distal portions), the pelvis, the spine, and the rib cage.

Conventional ultrasound imaging utilizes B-mode images. B-mode images are constructed by extracting an envelope of received scanned lines of radiofrequency (“RF”) signals using the Hilbert transformation. These envelopes are then decimated (causing a drop in the resolution) and converted to grayscale (intensity of each pixel is represented by 8 bits) to form the final B-mode image. The conversion to grayscale results in a drop in the dynamic range of the ultrasound data.

The use of ultrasound in computer aided orthopedic surgery has gained interest in the recent decade due to its relatively low cost and radiation-free nature. More particularly, A-mode ultrasound intra-operative registration has been used for computer aided orthopedic surgery and, in limited cases, in neurosurgery. Ultrasound-MRI registration has been developed utilizing B-mode ultrasound images. However, it has proven difficult to generate 3-D bone models having sufficient quality using conventional ultrasound technology due to limitations in the quality of the images.

Therefore, there is a need to develop improved apparatuses and methods that utilized ultrasound techniques to construct 3-D patient-specific bone and cartilage models.

The present invention overcomes the foregoing problems and other shortcomings, drawbacks, and challenges of high cost or high radiation exposure imaging modalities to generate a patient-specific model by ultrasound techniques. While the present invention will be described in connection with certain embodiments, it will be understood that the present invention is not limited to these embodiments. To the contrary, this invention includes all alternatives, modifications, and equivalents as may be included within the spirit and scope of the present invention.

In accordance with one embodiment of the present invention, a method of generating a 3-D patient-specific bone model is described. The method includes acquiring a plurality of raw radiofrequency (“RF”) signals from an A-mode ultrasound scan of the bone, which is spatially tracked in 3-D space. The bone contours are isolated in each of the plurality of RF signals and transformed into a point cloud. A 3-D patient-specific model of the bone is then optimized with respect to the point cloud.

According to another embodiment of the present invention, a method for 3-D reconstruction of a bone surface includes imaging the bone with A-mode ultrasound. A plurality of RF signals is acquired while imaging. Imaging of the bone is also tracked. A bone contour is extracted from each of the plurality of RF signals. Then, using the tracked data and the extracted bone contours, a point cloud representing the surface of the bone is generated. A generalized model of the bone is morphed to match the surface of the bone as represented by the point cloud.

In yet another embodiment of the present invention, a computer method for simulating a surface of a bone is described. The computer method includes executing a computer program in accordance with a process. The process includes extracting a bone contour from each of a plurality of A-mode RF signals. The extracted bone contours are transformed from a local frame of reference into a point cloud in a world-frame of reference. A generalized model of the bone is compared with the point cloud and, as determined from the comparing, the generalized model is deformed to match the point cloud.

Another embodiment of the present invention is directed to a computer program product that includes a non-transitory computer readable medium and program instructions stored on the computer readable medium. The program instructions, when executed by a process, cause the computer program product to isolate a bone contour from a plurality of RF signals. The plurality of RF signals being previously acquired from a reflected A-mode ultrasound beam. The bone contours are then transformed into a point cloud and used to optimize a 3-D model of the bone.

Still another embodiment of the present invention is directed to a computing device having a processor and a memory. The memory includes instructions that, when executed by the processor, cause the processor to isolate a bone contour from a plurality of RF signals. The plurality of RF signals being previously acquired from a reflected A-mode ultrasound beam. The bone contours are then transformed into a point cloud and used to optimize a 3-D model of the bone.

The various embodiments of the present invention are directed to methods of generating a 3-D patient-specific bone model. To generate the 3-D patient-specific model, a plurality of raw RF signals is acquired using A-mode ultrasound acquisition methodologies. A bone contour is then isolated in each of the plurality of RF signals and transformed into a point cloud. The point clouds may then be used to optimize a 3-D model of the bone such that the patient-specific model may be generated. Although the various embodiments of the invention are shown herein with respect to a human patient, persons having ordinary skill in the art will understand that embodiments of the invention may also be used to generate 3-D patient-specific bone models of animals (e.g., dogs, horses, etc.) such as for veterinarian applications.

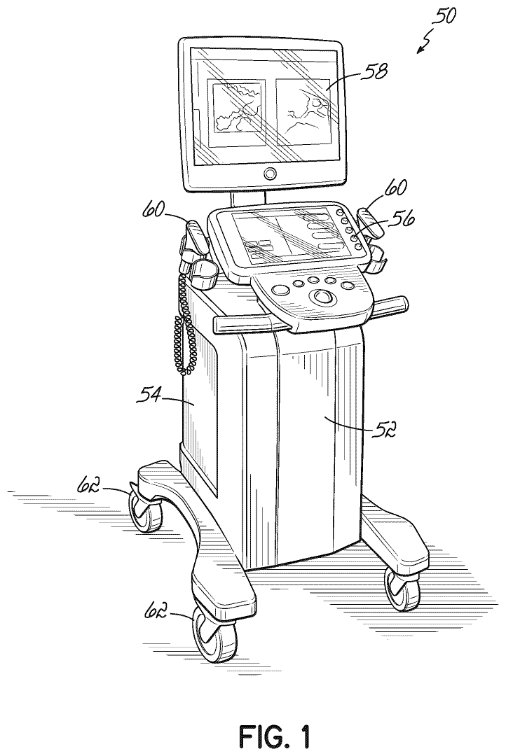

Turning now to the figures, and in particular to FIG. 1, one embodiment of an ultrasound instrument 50 for use with one or more embodiments of the present invention is shown. The ultrasound instrument 50 should be configurable such that the user may access acquired RF ultrasound data. One suitable instrument may, for example, include the diagnostic ultrasound model SonixRP by Ultrasonix Inc. (Richmond, British Columbia, Canada). The ultrasound instrument 50 includes a housing 52 containing a controller, (for example, a computer 54), an energy or power source (not shown), a user input device 56, an output device (for example, a monitor 58), and at least one ultrasound probe 60. The housing 52 may include caster wheels 62 for transporting the ultrasound instrument 50 within the medical facility.

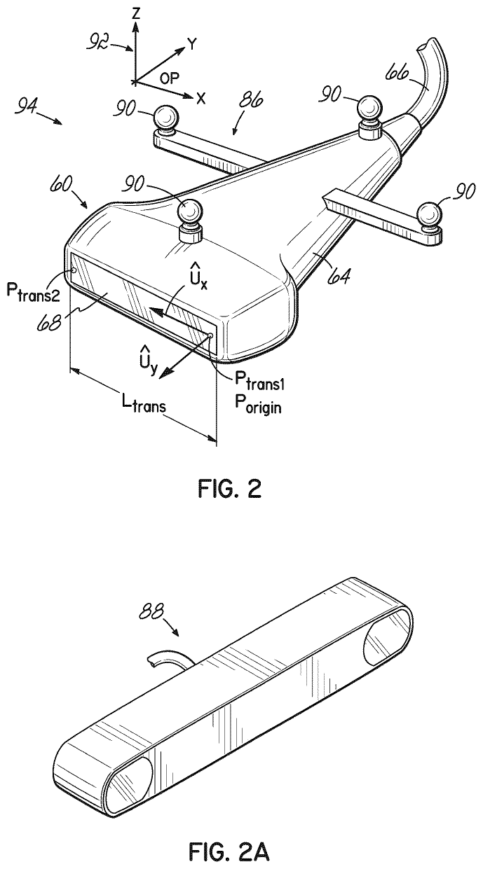

The at least one ultrasound probe 60 is configured to acquire ultrasound raw radiofrequency (“RF”) signals, and is shown in greater detail in FIG. 2. The ultrasound probe 60, such as the particular embodiment shown, may be a high resolution linear transducer with a center frequency of 7.5 MHz, as is conventionally used in musculoskeletal procedures. The sampling frequency used in digitizing ultrasound echo may be, for example, 20 MHz and must be at least twice the maximum ultrasound frequency. Generally, the ultrasound probe 60 includes a body 64 that is coupled to the ultrasound instrument housing 52 by a cable 66. The body 64 further includes a transducer array 68 configured to transmit an ultrasound pulse and to receive reflected ultrasound RF energy. The received RF echo is transmitted along the cable 66, to the computer 54 of the ultrasound instrument 50 for processing in accordance with an embodiment of the present invention.

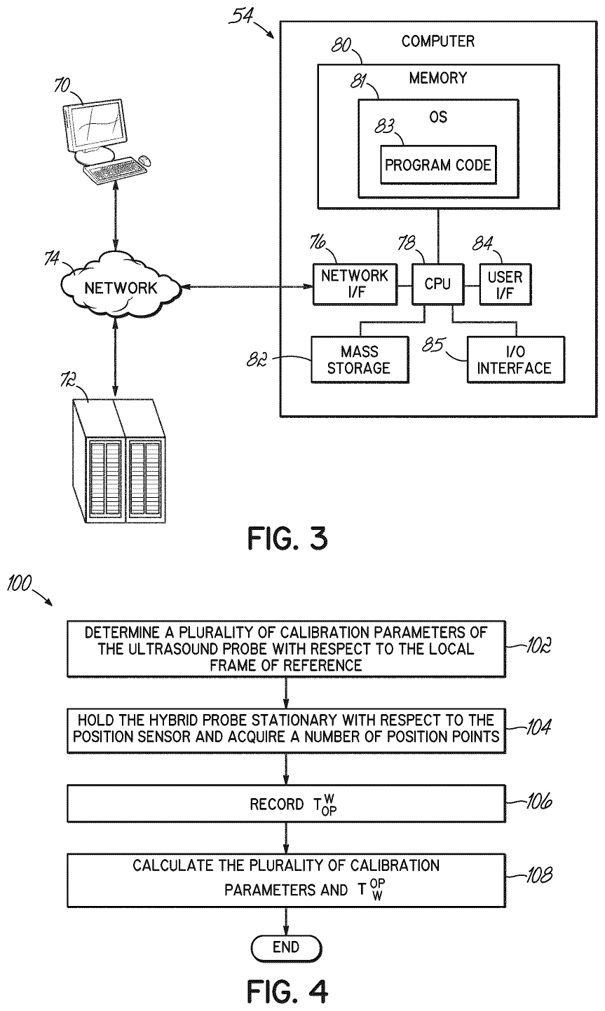

The computer 54 of the ultrasound instrument 50, as shown in FIG. 3, may be considered to represent any type of computer, computer system, computing system, server, disk array, or programmable device such as multi-user computers, single-user computers, handheld devices, networked devices, or embedded devices, etc. The computer 54 may be implemented with one or more networked computers 70 or networked storage devices 72 using one or more networks 74, e.g., in a cluster or other distributed computing system through a network interface 76 (illustrated as “NETWORK 1/F”). For brevity's sake, the computer 54 will be referred to simply as “computer,” although it should be appreciated that the term “computing system” may also include other suitable programmable electronic devices consistent with embodiments of the present invention.

The computer 54 typically includes at least one processing unit 78 (illustrated as “CPU”) coupled to a memory 80 along with several different types of peripheral devices, e.g., a mass storage device 82, the user interface 84 (illustrated as “User 1/F,” which may include the input device 56 and the monitor 58), the Network 1/F 76, and an Input/Output (10) interface 85 for coupling the computer 54 to additional equipment, such as the aforementioned ultrasound instrument 50. The memory 80 may include dynamic random access memory (“DRAM”), static random access memory (“SRAM”), non-volatile random access memory (“NVRAM”), persistent memory, flash memory, at least one hard disk drive, and/or another digital storage medium. The mass storage device 82 is typically at least one hard disk drive and may be located externally to the computer 54, such as in a separate enclosure or in one or more of the networked computers 70, one or more of the networked storage devices 72 (for example, a server).

The CPU 78 may be, in various embodiments, a single-thread, multi-threaded, multi-core, and/or multi-element processing unit (not shown). In alternative embodiments, the computer 54 may include a plurality of processing units that may include single-thread processing units, multi-threaded processing units, multi-core processing units, multi-element processing units, and/or combinations thereof. Similarly, the memory 80 may include one or more levels of data, instruction, and/or combination caches, with caches serving the individual processing unit or multiple processing units (not shown).

The memory 80 of the computer 54 may include an operating system 81 (illustrated as “OS”) to control the primary operation of the computer 54 in a manner known in the art. The memory 80 may also include at least one application, component, algorithm, program, object, module, or sequence of instructions, or even a subset thereof, will be referred to herein as “computer program code” or simply “program code” 83. Program code 83 typically comprises one or more instructions that are resident at various times in the memory 80 and/or the mass storage device 82 of the computer 54, and that, when read and executed by the CPU 78, causes the computer 54 to perform the steps necessary to execute steps or elements embodying the various aspects of the present invention.

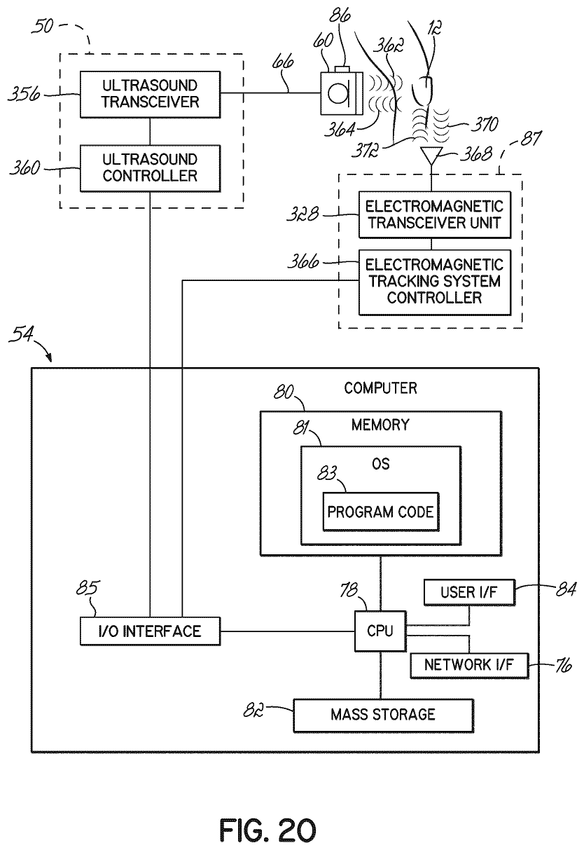

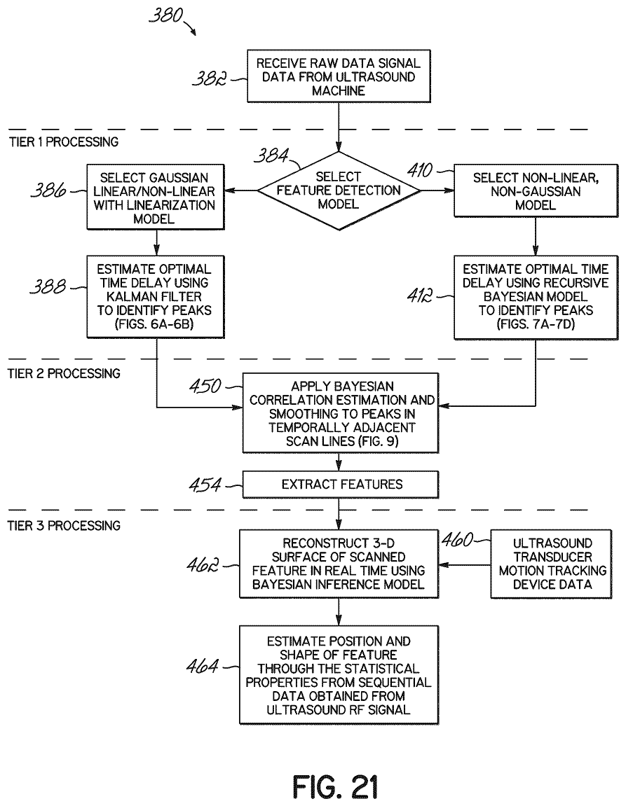

The I/O interface 85 is configured to operatively couple the CPU 78 to other devices and systems, including the ultrasound instrument 50 and an optional electromagnetic tracking system 87 (FIG. 20). The I/O interface 85 may include signal processing circuits that condition incoming and outgoing signals so that the signals are compatible with both the CPU 78 and the components to which the CPU 78 is coupled. To this end, the I/O interface 85 may include conductors, analog-to-digital (A/D) and/or digital-to-analog (D/A) converters, voltage level and/or frequency shifting circuits, optical isolation and/or driver circuits, and/or any other analog or digital circuitry suitable for coupling the CPU 78 to the other devices and systems. For example, the I/O interface 85 may include one or more amplifier circuits to amplify signals received from the ultrasound instrument 50 prior to analysis in the CPU 78.

Those skilled in the art will recognize that the environment illustrated in FIG. 3 is not intended to limit the present invention. Indeed, those skilled in the art will recognize that other alternative hardware and/or software environments may be used without departing from the scope of the present invention.

Returning again to FIG. 2, the ultrasound probe 60 has mounted thereto a tracking marker 86, which, for purposes of illustration only, is shown as an optical marker, configured to spatially register the motion of the ultrasound probe 60 during signal acquisition. The tracking marker 86 may be comprised of a plurality of reflective portions 90, which are described in greater detail below. The tracked probe constitutes a hybrid probe 94. In other embodiments, the tracking marker and associated system may be electromagnetic, RF, or any other known 3-D tracking system.

The optical tracking marker 86 is operably coupled to a position sensor 88, one embodiment of which is shown in FIG. 2A. In use, the position sensor 88 emits energy (for example, infrared light) in a direction toward the optical tracking marker 86. Reflective portions 90 of the optical tracking marker 86 reflect the energy back to the position sensor 88, which then triangulates the 3-D position and orientation of the optical tracking marker 86. One example of a suitable optical tracking system is the Polaris model manufactured by Northern Digital Inc. (Waterloo, Ontario, Canada).

The optical tracking marker 86 is rigidly attached to the ultrasound probe 60 and is provided a local coordinate frame of reference (“local frame” 92). Additionally, the ultrasound probe 60 is provided another local coordinate frame of reference (“ultrasound frame”). For the sake of convenience, the combination optical tracking marker 86 with the ultrasound probe 60 is referred to as the “hybrid probe” 94. The position sensor 88, positioned away from the hybrid probe 94, determines a fixed world coordinate frame (“world frame”). Operation of the optical tracking system (the optical tracking marker 86 with the position sensor 88) with the ultrasound probe 60, once calibrated, is configured to determine a transformation between the local and ultrasound coordinate frames.

Turning now to FIG. 4, with continued reference to FIG. 2, a method 100 of calibrating the optical tracking system according to one embodiment of the present invention is described. To calibrate the optical tracking marker 86 with the position sensor 88, for real-time tracking of the hybrid probe 94, a homogeneous transformation T_{OP}^{W} between the local frame, OP, and the world frame, W, is needed. The calibration method 100 begins with determining a plurality of calibration parameters (Block 102). In the particular illustrative example, four parameters are used and include: P_{trans-origin}, i.e., a point of origin on the transducer array 68; Lₜᵣₐₙₛ, i.e., a length of the transducer array 68; ûₓ, i.e., a unit vector along the length of the transducer array 68; 4) û_{y}, i.e., a unit vector in a direction that is perpendicular to the length of the transducer array 68. These calibration points and vectors are relative to the local frame 92 (“OP”).

The hybrid probe is held in a fixed position while the position sensor 88 optical camera acquires a number of position points, including, for example: Pₜᵣₐₙₛ₁, i.e., a first end of the transducer array 68; Pₜᵣₐₙₛ₂, i.e., a second end of the transducer array 68; and Po, i.e., a point on the transducer array 68 that is not collinear with Pₜᵣₐₙₛ₁ and Pₜᵣₐₙₛ₂ (Block 104). The homogeneous transformation between OP and W, T_{OP}^{W}, is then recorded (Block 106). The plurality of calibration parameters are then calculated (Block 108) from the measured number of points and the transformation, T_{OP}^{W}, as follows:

$${T}_{W}^{OP} = {( {T}_{OP}^{W} )}^{- 1} \qquad ( 1 )$$

$${P}_{trans - origin} = {T}_{W}^{OP} {P}_{trans 1} \qquad ( 2 )$$

$${L}_{trans} = ‖ {P}_{trans 2} - {P}_{trans 1} ‖ \qquad ( 3 )$$

$${\hat{u}}_{x} = {T}_{W}^{OP} \frac{{P}_{trans 2} - {P}_{trans 1}}{‖ {P}_{trans 2} - {P}_{trans 1} ‖} \qquad ( 4 )$$

$${\hat{u}}_{y} = \frac{( {P}_{plane} - {P}_{trans 1} ) \times ( {P}_{trans 2} - {P}_{trans 1} )}{‖ ( {P}_{plane} - {P}_{trans 1} ) \times ( {P}_{trans 2} - {P}_{trans 1} ) ‖} \qquad ( 5 )$$

With the plurality of calibration parameters determined, the hybrid probe 94 may be used to scan a portion of a patient's musculoskeletal system while the position sensor 88 tracks the physical movement of the hybrid probe 94.

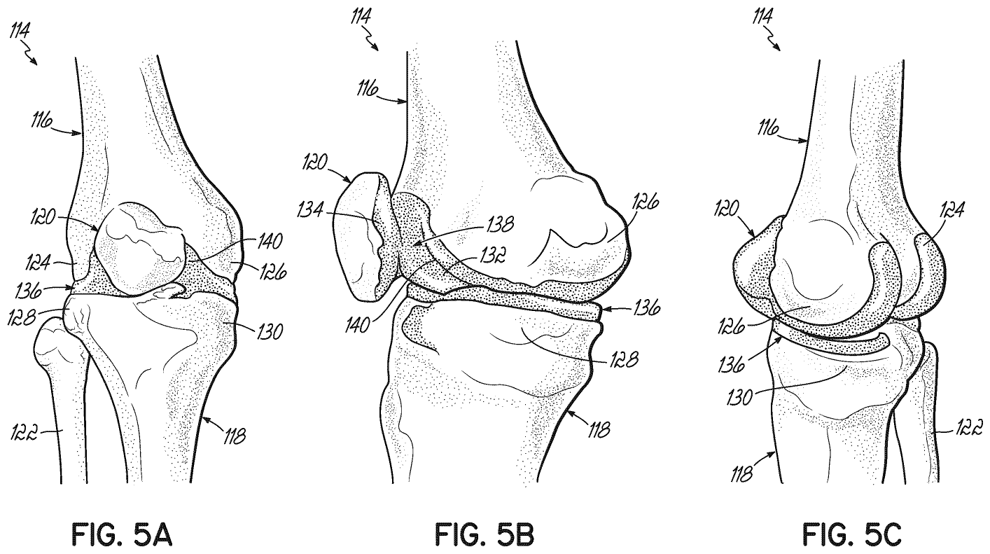

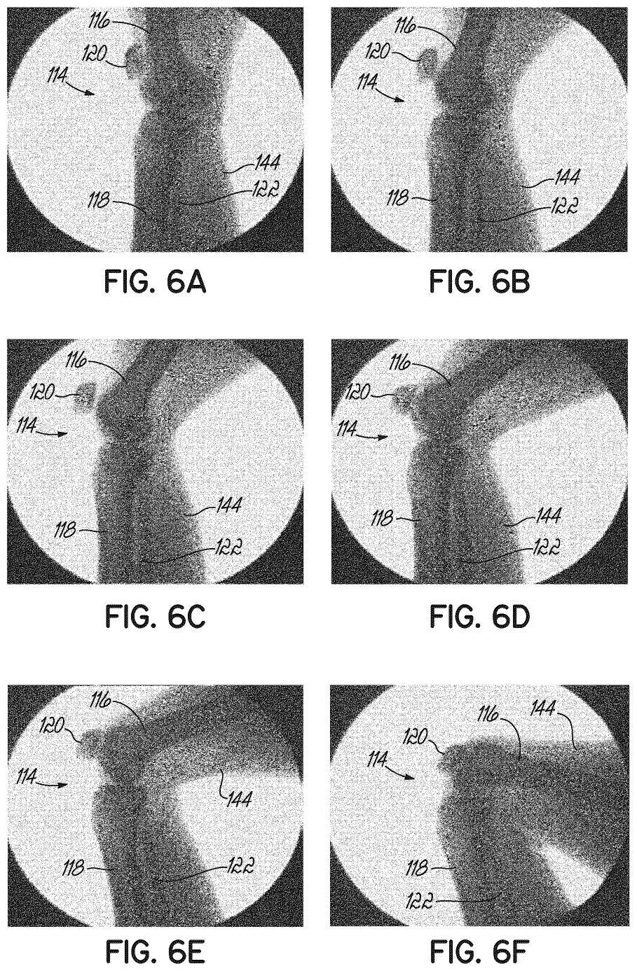

Because of the high reflectivity and attenuation of bone to ultrasound, ultrasound energy typically does not penetrate bone tissues to any significant degree. Therefore, soft tissues lying behind bone cannot be imaged and poses a challenge to ultrasound imaging of a joint. For example, as shown in FIGS. 5A-5C, the knee joint 114 is formed of three articulating bones: the femur 116, the tibia 118, and the patella 120, with the fibula 122 shown as environment. These bones 116, 118, 120 articulate together in two sub-joints: (1) the tibio-femoral joint 136 is formed by the articulation of the femur 116 with the tibia 118 at the respective condyles 124, 126, 128, 130 and (2) the patello-femoral joint 138 is formed by the articulation of the patella 120 with the femur 116 at the patellar surface 132 of the femur 116 and the articular surface 134 of the patella 120. During flexion-extension motions of the knee joint 114, portions of one or more articulating surfaces of the bones 116, 118, 120 are visible to the ultrasound beam, while other articulating surfaces are occluded. FIGS. 6A-6F include various fluoroscopic images of one patient's knee joint 114, showing the articulating surfaces at a plurality of degrees of flexion.

To acquire ultrasound images of a majority of the articulating surfaces, at least two degrees of flexion are required, including, for example, a full extension (FIG. 6A) and a deep knee bend (FIG. 6F) (or 90° flexion (FIG. 6E) if a deep knee bend is too difficult for the patient to achieve). That is, when the knee joint 114 is in the full extension (FIG. 6A), the posterior portions of the distal femur 116 and the proximal tibia 118 are accessible to the ultrasound beam. When the knee joint 114 is in the deep knee bend (FIG. 6F), the anterior surface of the distal femur 116, the trochlear grove 140, most of the inferior surface of the femoral condyles 124, 126, the anterior superior surface of the tibia 118, and the anterior surface of the tibia 11 8 are accessible to the ultrasound beam. Both the medial and lateral parts of the femur 116 and tibia 118 are visible at all flexion angles of the knee joint 114.

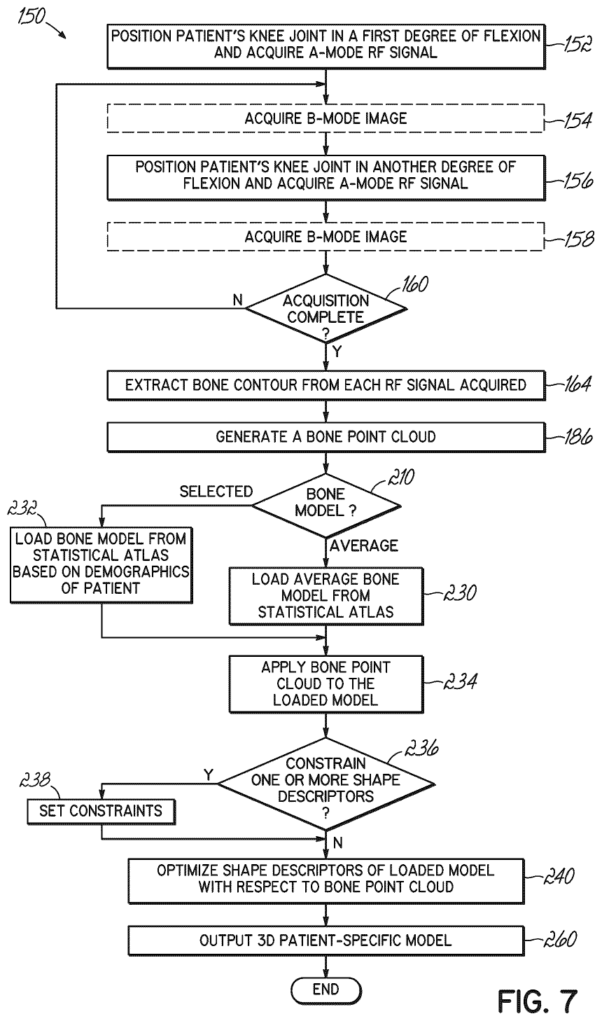

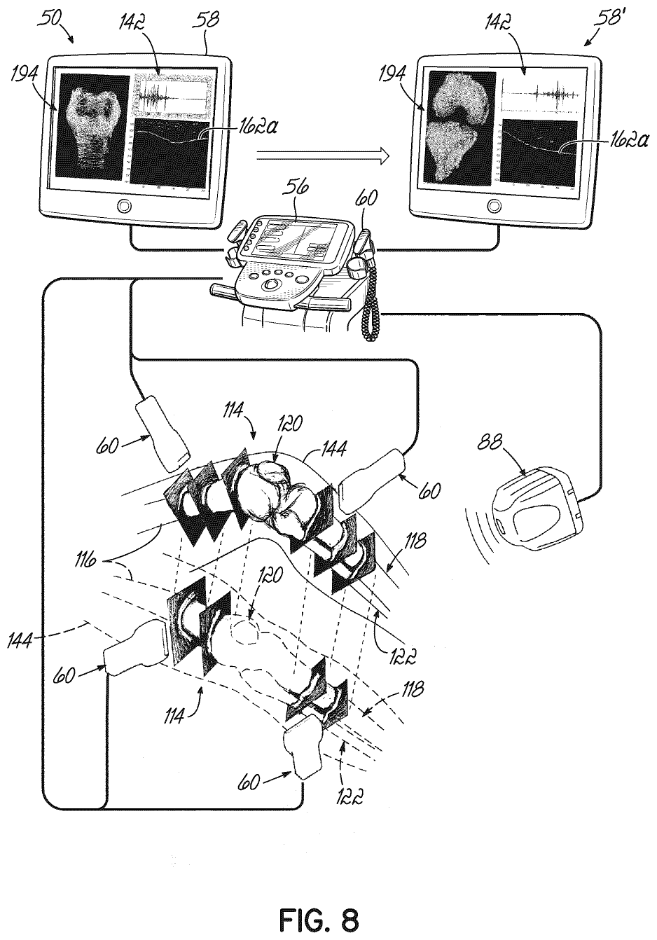

Turning now to FIG. 7, one method 150 of acquiring data for construction of a 3-D patient-specific bone model in accordance with aspects of the invention is described. The method begins with acquiring a plurality of RF signals from an A-mode ultrasound beam scan of a bone. To acquire the RF signals for creating the 3-D patient-specific model of the knee joint 114, the patient's knee joint 114 is positioned and held in one of the two or more degrees of flexion (Block 152). The hybrid probe 94 is positioned, at two or more locations, on the patient's epidermis 144 adjacent to the knee joint 114 for acquisition of the A-mode RF signal 142, one example, as shown in FIG. 8. Although the acquired signal includes a plurality of RF signals, for convenience, the RF signals are sometimes referred to herein in singular form.

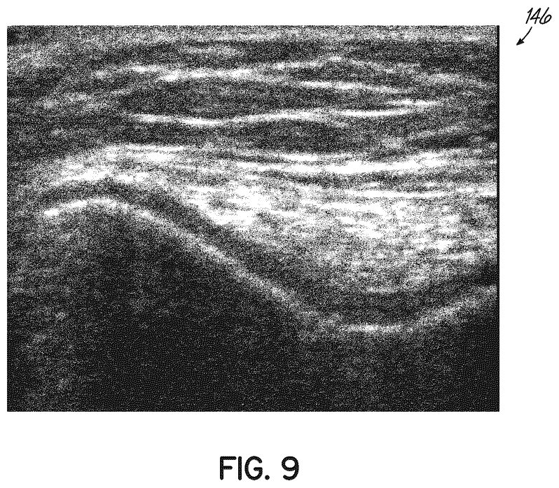

As shown in FIG. 8, with continued reference to FIG. 7, the position of the patient's knee joint 114 is held stationary to avoid motion artifacts during image acquisition. Should motion occur, scans may be automatically aligned to the statistically-most likely position given the data acquired. Furthermore, holding the knee stationary and compensating for movement removes the need for invasive fiducial bone markers or high-error skin markers. In some embodiments, B-mode images, similar to the one shown in FIG. 9, may also be processed from the gathered data (Block 154) for subsequent visualization and overlain with the bone contours, as described in detail below.

When the RF signal 142, and if desired B-mode image, acquisition is complete for the first degree of flexion, the patient's knee 114 is moved to another degree of flexion and the reflected RF signal 142 acquired (Block 156). Again, if desired, the B-mode image may also be acquired (Block 158). The user then determines whether acquisition is complete or whether additional data is required (Block 160). That is, if visualization of a desired surface of one or more bones 116, 118, 120 is occluded (“NO” branch of decision block 160), then the method returns to acquire additional data at another degree of flexion (Block 156). If the desired bone surfaces are sufficiently visible (“YES” branch of decision block 160), then the method 150 continues.

FIG. 8 illustrates acquisition of the RF signal 142 in yet another manner. That is, while the patient's leg is in full extension (shown in phantom), the hybrid probe 94 is positioned at two or more locations on the patient's epidermis 144 adjacent to the knee joint 114. The patient's leg is then moved to a second degree of flexion (90° flexion is shown in solid) and the hybrid probe 94 again positioned at two or more locations on the patient's epidermis 144. All the while, the position sensor 88 tracks the location of the hybrid probe 94 in the 3-D space. Resultant RF signal profiles, bone models, bone contours, and so forth may be displayed on the monitor 58 during and the monitor 58′ after the model reconstruction.

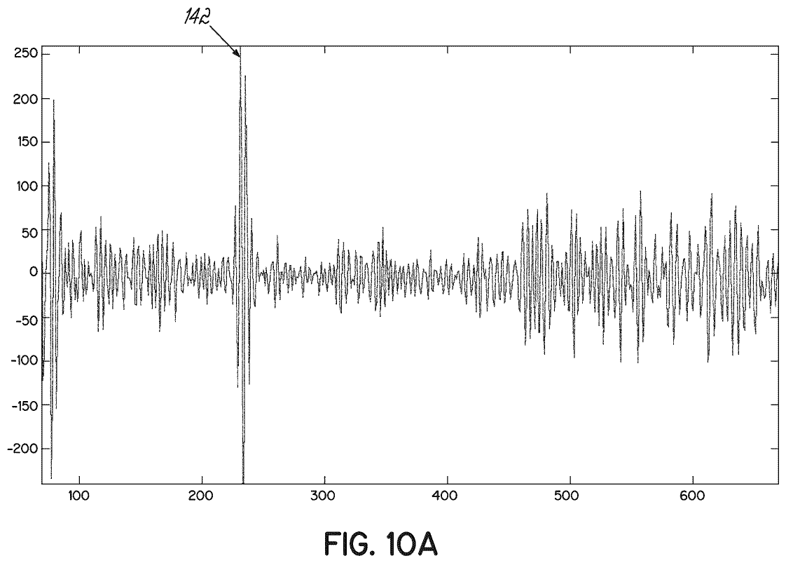

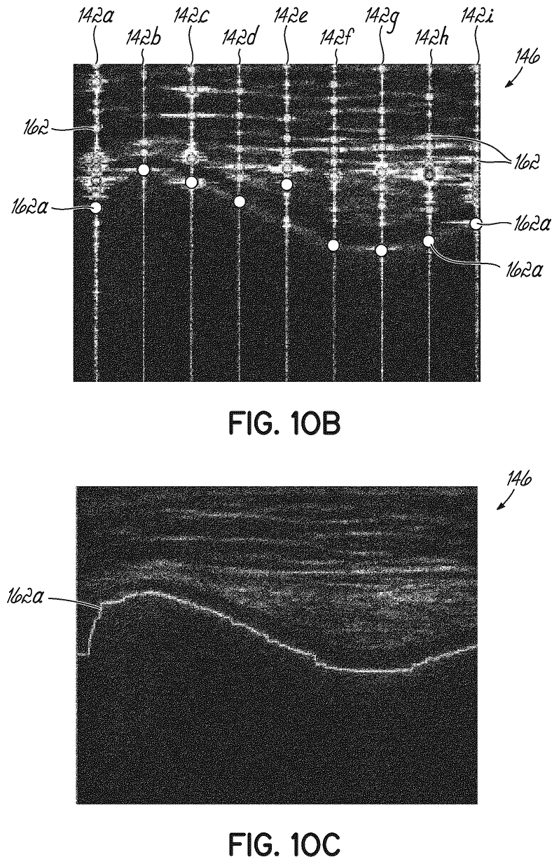

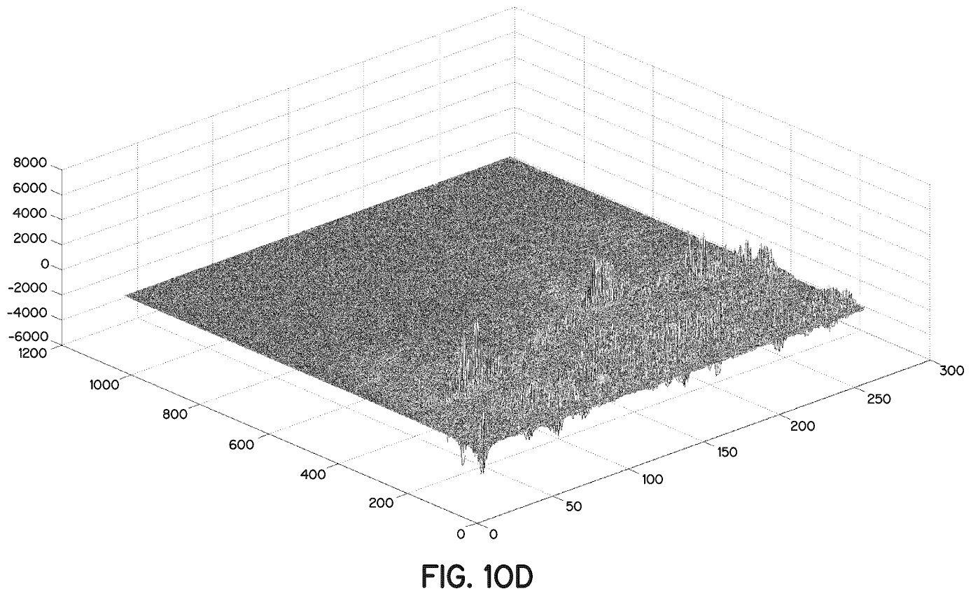

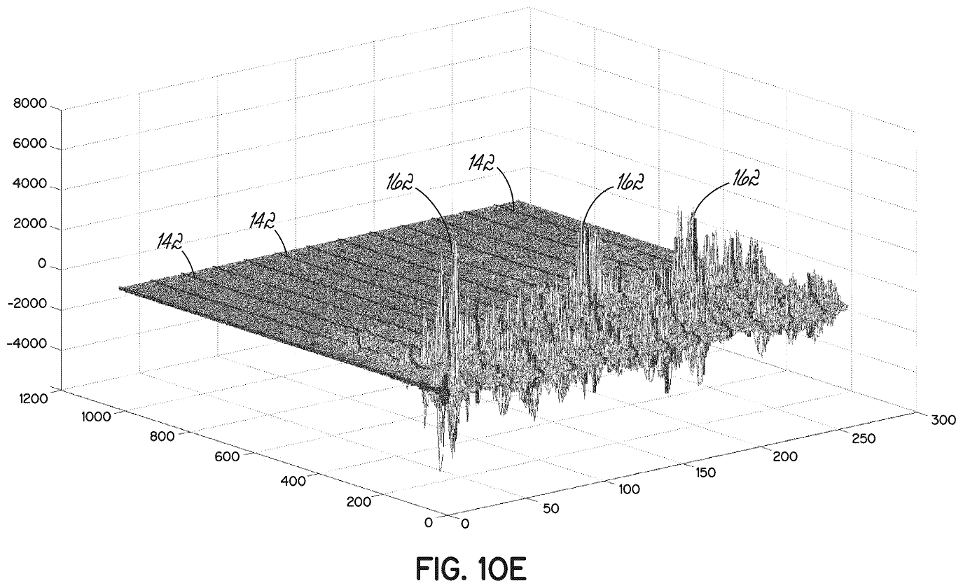

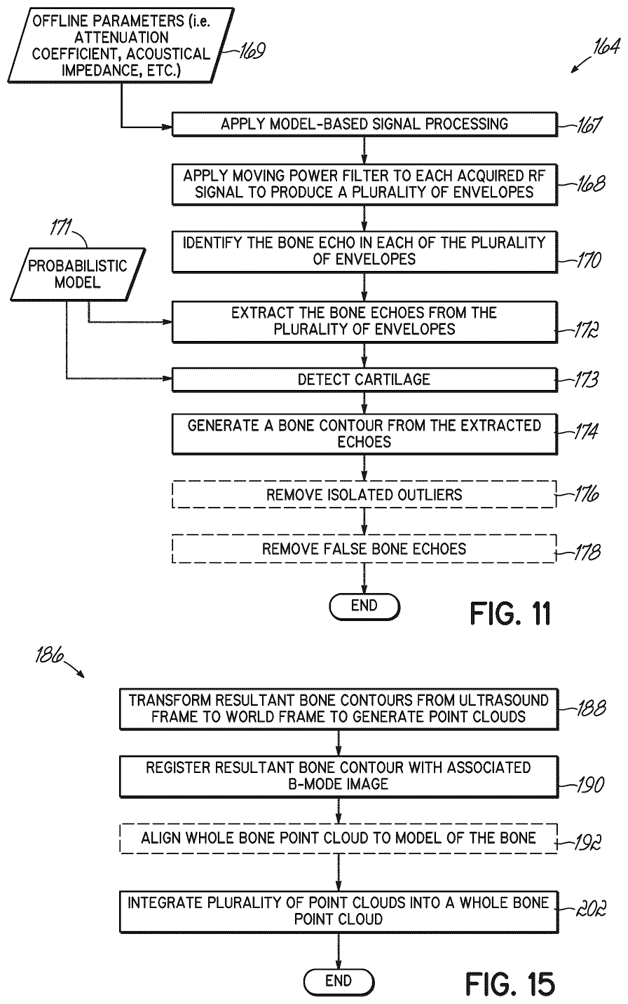

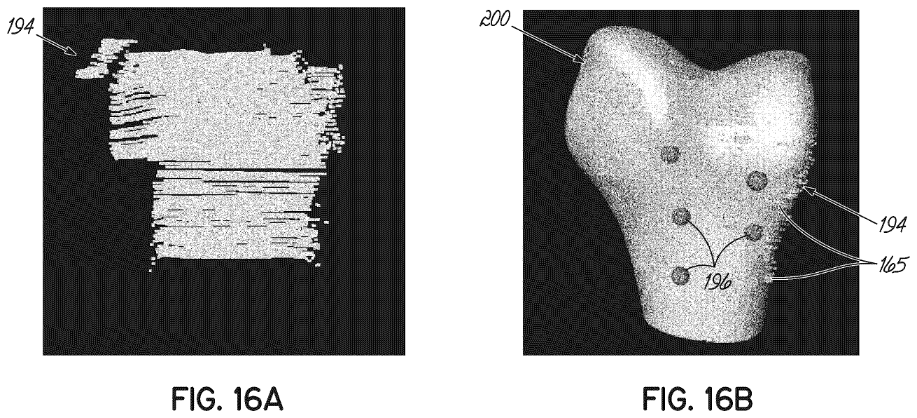

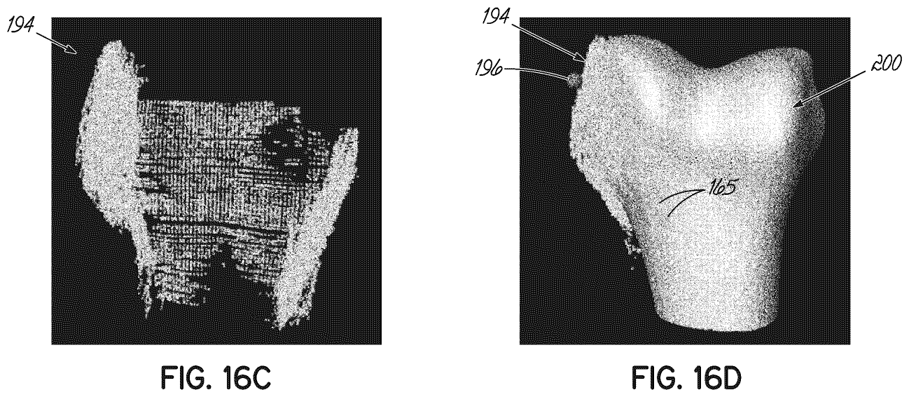

After all data and RF signal acquisition is complete, the computer 54 is operated to automatically isolate that portion of the RF signal, i.e., the bone contour, from each of the plurality of RF signals. In that regard, the computer 54 may sample the echoes comprising the RF signals to extract a bone contour for generating a 3-D point cloud 165 (FIG. 16B) (Block 164). More specifically, and with reference now to FIGS. 10A-10E and 11, and with continued reference to FIGS. 7-9, one method of extracting the bone contours from each of the RF signal 142 is shown. FIG. 10A illustrates one exemplary, raw RF signal 142 as acquired by one transducer comprising the transducer array 68 of the ultrasound probe portion of the hybrid probe 94. Each acquired raw, RF signal includes a number of echoes 162, wherein the echoes 162 may be isolated, partially overlapping, or fully overlapping. Each of the plurality of echoes originates from a reflection of at least a portion of the ultrasound energy at an interface between two tissues having different reflection and/or attenuation coefficients, as described in greater detail below.

FIGS. 10B and 10C illustrate an ultrasound frame 146 having select ones of the raw RF signals 142 with some echoes 162 identified. FIGS. 10D and 10E are 3-D renderings of 2D images taken from an ultrasound frame 146 with select ones of the RF signals 142 identified in FIG. 10E.

Referring specifically now to FIG. 11, the method of extracting the bone contour 162a begins with a model-based signal processing approach incorporating a priori knowledge of an underlying physical problem into a signal processing scheme. In this way, the computer 54 may process the RF signal 142 and remove some preliminary noise based on an estimated, or anticipated, result. For example, with ultrasound signal acquisition, the physical problem is represented by the governing waveform equation, such as described in VARSLOT T, et al., “Computer Simulation of Forward Wave Propagation in Soft Tissue,” IEEE Transactions on Ultrasonics, Ferroelectrics, and Frequency Control, 1473-1482:52(9), September 2005, the disclosure of which is incorporated herein by reference, in its entirety. The wave equation describes the propagation behavior of the ultrasonic wave in a heterogeneous medium. The solution to the wave equation may be represented as a state-space model-based processing scheme, such as described in CHEN Z, et al., “Bayesian Filtering: From Kalman Filters to Particle Filters, and Beyond,” Statistics, 1-69. In accordance with one embodiment of the present invention, a general solution to the model-based ultrasound wave estimator problem is developed using Bayesian estimators (e.g., maximum a posteriori), which leads to a nonlinear model-based design.

The model-based signal processing of the RF signal 142 begins with enhancing the RF signal by applying the model-based signal processing (here, the Bayesian estimator) (Block 167). To apply the Bayesian estimator, offline measurements are first collected from phantoms, cadavers, and/or simulated tissues to estimate certain unknown parameters, for example, an attenuation coefficient (i.e., absorption and scattering) and an acoustic impedance (i.e., density, porosity, compressibility), in a manner generally described in VARSLOT T (refer above), the disclosure of which is incorporated herein by reference, in its entirety. The offline measurements (Block 169) are input into the Bayesian estimator and the unknown parameters are estimated as follows:

z=h(x)+v (6)

P(t)=e^{(-βt}^{²}⁾·cos(2π·f₀·t) (7)

Where h is the measurement function that models the system and v is the noise and modeling error. In modeling the system, the parameter, x, that best fits the measurement, z, is determined. For example, the data fitting process may find an estimate of \hat{x} that best fits the measurement of z by minimizing some error norm, ∥∈∥, of the residual, where:

ε=z−h(\hat{x}) (8)

For ultrasound modeling, the input signal, z, is the raw RF signal from the offline measurements, the estimate h(\hat{x}) is based on the state space model with known parameters of the offline measurements (i.e., density, etc.). The error, v, may encompass noise, unknown parameters, and modeling errors in an effort to reduce the effect of v by minimizing the residuals and identifying the unknown parameters form repeated measurements. Weighting the last echo within a scan line by approximately 99%, as bone, is one example of using likelihood in a Bayesian framework. A Kalman filter may alternatively be used, which is a special case of the recursive Bayesian estimation, in which the signal is assumed to be linear and have a Gaussian distribution.

It would be readily appreciated that the illustrative use of the Bayesian model here is not limiting. Rather, other model-based processing algorithms or probabilistic signal processing methods may be used within the spirit of the present invention.

With the model-based signal processing complete, the RF signal 142 is then transformed into a plurality of envelopes to extract the individual echoes 162 existing in the RF signal 142. Each envelope is determined by applying a moving power filter to each RF signal 142 (Block 168) or other suitable envelope detection algorithm. The moving power filter may be comprised of a moving kernel of a length that is equal to the average length of an individual ultrasound echo 162. With each iteration of the moving kernel, the power of the RF signal 142 at the instant kernel position is calculated. One exemplary kernel length may be 20 samples; however, other lengths may also be used. The value of the RF signal 142 represents the value of the signal envelope at that position of the RF signal 142. Given a discrete-time signal, X, having a length, N, each envelope, V, using a moving power filter having length, L, is defined by:

$${γ}_{k} = {\sum }_{i = k - \frac{L}{2}}^{k + \frac{L}{2}} {X}_{i}^{2} ∀ k ∈ [ \frac{L}{2} , N - \frac{L}{2} - 1 ] \qquad ( 9 )$$

In some embodiments, this and subsequent equations use a one-sided filter of varying length for the special cases of the samples before the sample

$$i \frac{L}{2} ( left - sided filter ) ,$$

and after the sample

$$N - \frac{L}{2} - 1 ( right - sided filter ) .$$

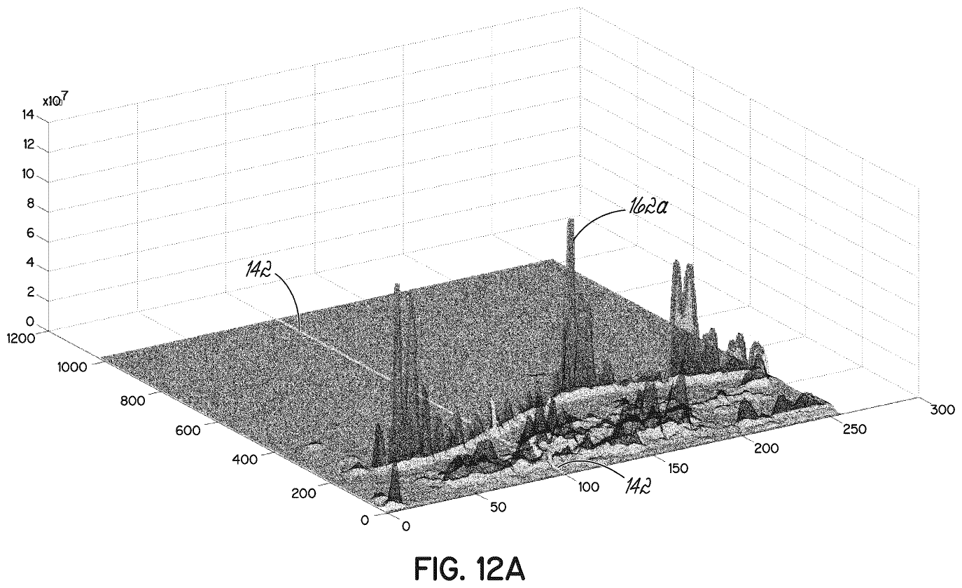

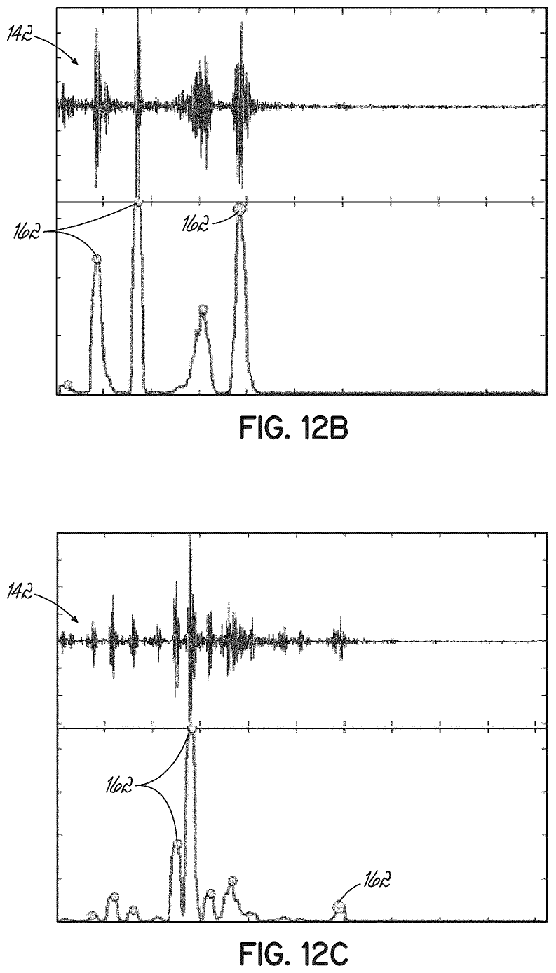

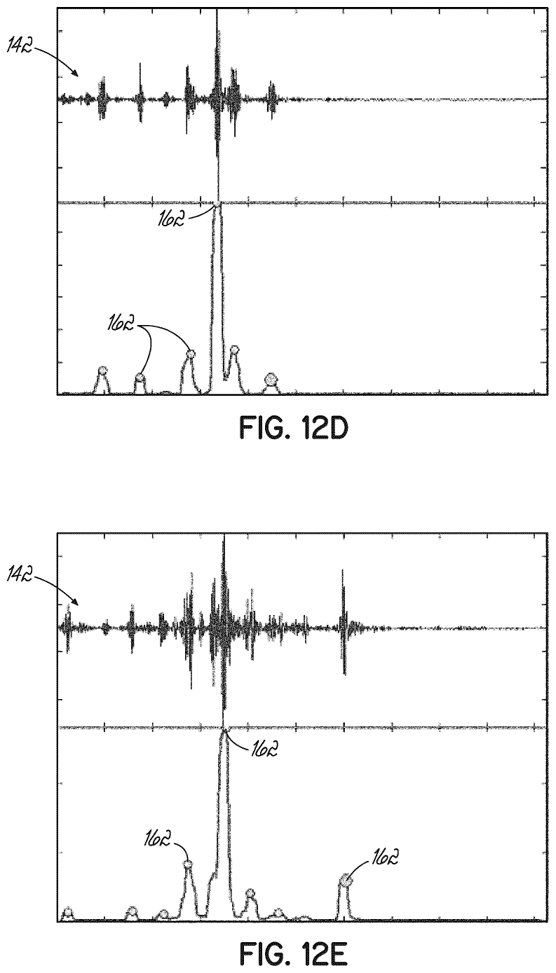

Each envelope produced by the moving power filter, shown in FIG. 10B, includes a plurality of local peaks (identified in FIG. 10B as enlarged dots at the intersection of each envelope with an echo 162), each being a clear representation of the individual echoes 162 existing in the acquired RF signal 142 for the various tissue interfaces. As an example of such process, FIGS. 12A-12D more clearly illustrate the RF signal 142 (top in each figure) at four iterations of the kernel of the moving power filter as well as the corresponding envelope (bottom in each figure). Individual echoes 162 in each envelope are again identified with an enlarged dot.

Of the plurality of echoes 162 in the RF signal 142, one echo 162 is of particular interest, e.g., the echo corresponding to the bone-soft tissue interface. This bone echo (hereafter referenced as 162a) is generated by the reflection of the ultrasound energy at the surface of the scanned bone. More particularly, the soft tissue-bone interface is characterized by a high reflection coefficient of 43%, which means that 43% of the ultrasound energy reaching the surface of the bone is reflected back to the transducer array 68 of the ultrasound probe 60 (FIG. 2). This high reflectivity gives bone the characteristic hyper-echoic appearance in an ultrasound image.

Bone is also characterized by a high attenuation coefficient of the applied RF signal (6.9 db/cm/mHz for trabecular bone and 9.94 db/cm/mHz for cortical bone). At high frequencies, such as those used in musculoskeletal imaging (that is, in the range of 7-14 MHz), the attenuation of bone becomes very high and the ultrasound energy ends at the surface of the bone. Therefore, an echo 162a corresponding to the soft-tissue-bone interface is the last echo 162a in the RF signal 142. The bone echo 162a is identified by selecting the last echo having a normalized envelope amplitude (with respect to a maximum value existing in the envelope) above a preset threshold (Block 170).

The bone echoes 162a are then extracted from each frame 146 (Block 172) and used to generate the bone contour existing in that RF signal 142 and as shown in FIG. 10C (Block 174). In extracting the bone echoes, a probabilistic model (Block 171) may be input and applied to the RF signals 142 of each frame 146. The probabilistic model (Block 171) may further be used in detecting cartilage within the envelopes of the RF signals 142 (Block 173). While the probabilistic signal processing method may include the Bayesian estimator described previously, in still other embodiments, the signal processing may be, a maximum likelihood ratio, neural network, or a support vector machine (“SVM”), for example, with the latter of which is further described below.

Prior to implementing the SVM, the SVM may be trained to detect cartilage in RF signals. One such way of training the SVM includes information acquired from a database comprising of MRI images and/or RF ultrasound images to train the SVM to distinguish between echoes associated with cartilage from the RF signals 142, and from within the noise or in ambiguous soft tissue echoes. In constructing the database in accordance with one embodiment, knee joints from multiple patients are imaged using both MRI and ultrasound. A volumetric MRI image of each knee joint is reconstructed, processed, and the cartilage and the bone tissues are identified and segmented. The segmented volumetric MRI image is then registered with a corresponding segmented ultrasound image (wherein bone tissue is identified). The registration provides a transformation matrix that may then be used to register the raw RF signals 142 with a reconstructed MRI surface model.

After the raw RF signals 142 are registered with the reconstructed MRI surface model, spatial information from the volumetric MRI images with respect to the cartilage tissue may be used to determine the location of a cartilage interface on the raw RF signal 142 over the articulating surfaces of the knee joint.

The database of all knee joint image pairs (MRI and ultrasound) is then used to train the SVM. Generally, the training includes loading all raw RF signals, as well as the location of the bone-cartilage interface of each respective RF signal. The SVM may then determine the location of the cartilage interface in an unknown, input raw RF signal. If desired, a user may choose from one or more kernels to maximize a classification rate of the SVM.

In use, the trained SVM receives a reconstructed knee joint image of a new patient as well as the raw RF signals. The SVM returns the cartilage location on the RF signal data, which may be used, along with the tracking information from the tracking system (e.g., the optical tracking marker 86 and the position sensor 88) to generate 3-D coordinates for each point on the cartilage interface. The 3-D coordinates may be triangulated and interpolated to form a complete cartilage surface.

Referring still to FIG. 11, the resultant bone contours may be noisy and require filtering to remove echoes 162 that may be falsely detected as the bone echo 162a. Falsely detected echoes 162 may originate from one of at least two sources: (1) an isolated outlier echoes and (2) a false bone echoes. Furthermore, some images may not include a bone echo 162a; therefore any detected echo 162 is noise and should be filtered out. Therefore, proper determination of the preset threshold or filtering algorithm may prevent the false selection of a falsely detected echo 162.

Isolated outliers are those echoes 162 in the RF signal 142 that correspond to a tissue interface that is not the soft-tissue-bone interface. Selection of the isolated outliers may occur when the criterion is set too high. If necessary, the isolated outliers may be removed (Block 176) by applying a median filter to the bone contour. That is, given a particular bone contour, X, having a length, N, with a median filter length, L, the median-filter contour, Yₖ, is:

$${Y}_{k} = Median [ {X}_{k - \frac{L}{2}} , {X}_{k + \frac{L}{2}} ] ∀ k ∈ [ \frac{L}{2} , N - \frac{L}{2} - 1 ] \qquad ( 10 )$$

False bone echoes are those echoes 162 resulting from noise or a scattering echo, which result in a detected bone contour in a position where no bone contour exists. The false bone echoes may occur when an area that does not contain a bone is scanned, the ultrasound probe 60 is not oriented substantially perpendicular with respect to the bone surface, the bone lies deeper than a selected scanning depth, the bone lies within the selected scanning depth but its echo is highly attenuated by the soft tissue overlying the bone, or a combination of the same. Selection of the false bone echoes may occur when the preset threshold is too low.

Frames 146 containing false bone echoes should be removed. One such method of removing the false bone echoes (Block 178) may include applying a continuity criteria. That is, because the surface of the bone has a regular shape, the bone contour, in the two-dimensions of the ultrasound image, should be continuous and smooth. A false bone echo will create a non-continuity, and exhibits a high degree of irregularity with respect to the bone contour.

One manner of filtering out false bone echoes is to apply a moving standard deviation filter; however, other filtering methods may also be used. For example, given the bone contour, X, having a length, N, with a median filter length, L, the standard deviation filter contour:

$${Y}_{k} = \sqrt{\frac{1}{L - 1} {\sum }_{i = k - \frac{L}{2}}^{i = k - \frac{L}{2}} {( {X}_{i} - \overline{X} )}^{2}} ∀ k ∈ [ \frac{L}{2} , N - \frac{L}{2} - 1 ] \qquad ( 11 )$$

Where Yₖ is the local standard deviation of the bone contour, which is a measure of the regularity and continuity of the bone contour. Segments of the bone contour including a false bone echo are characterized by a higher degree of irregularity and have a high Yₖ value. On the other hand, segments of the bone contour including only echoes resulting from the surface of the bone are characterized by high degree regularity and have a low Yₖ value.

A resultant bone contour 180, resulting from applying the moving median filter and the moving standard deviation filter, includes a full length contour of the entire surface of the bone, one or more partial contours of the entire surface, or contains no bone contour segments.

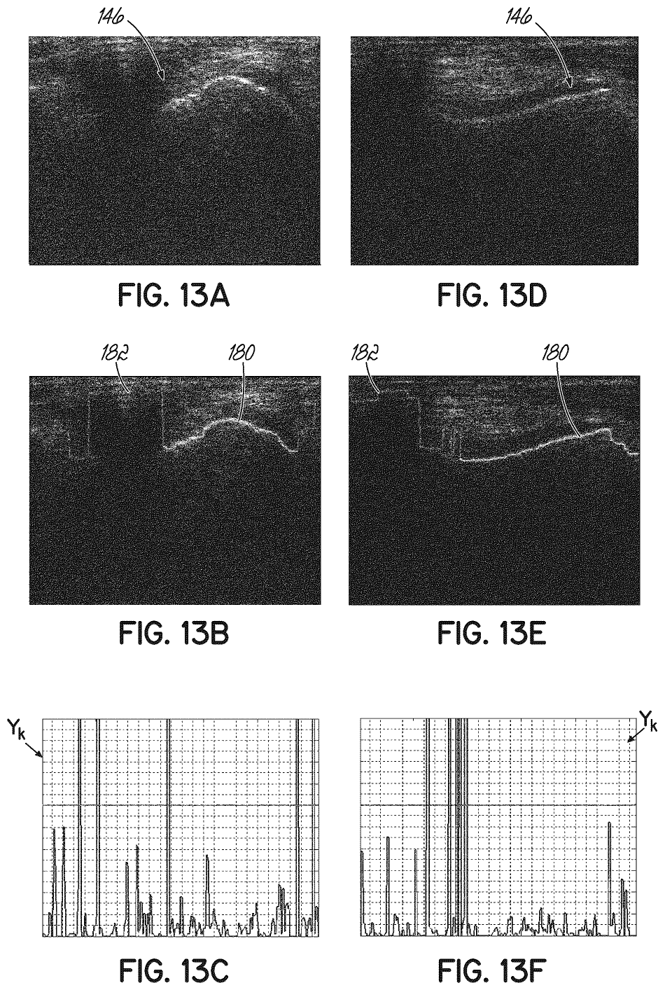

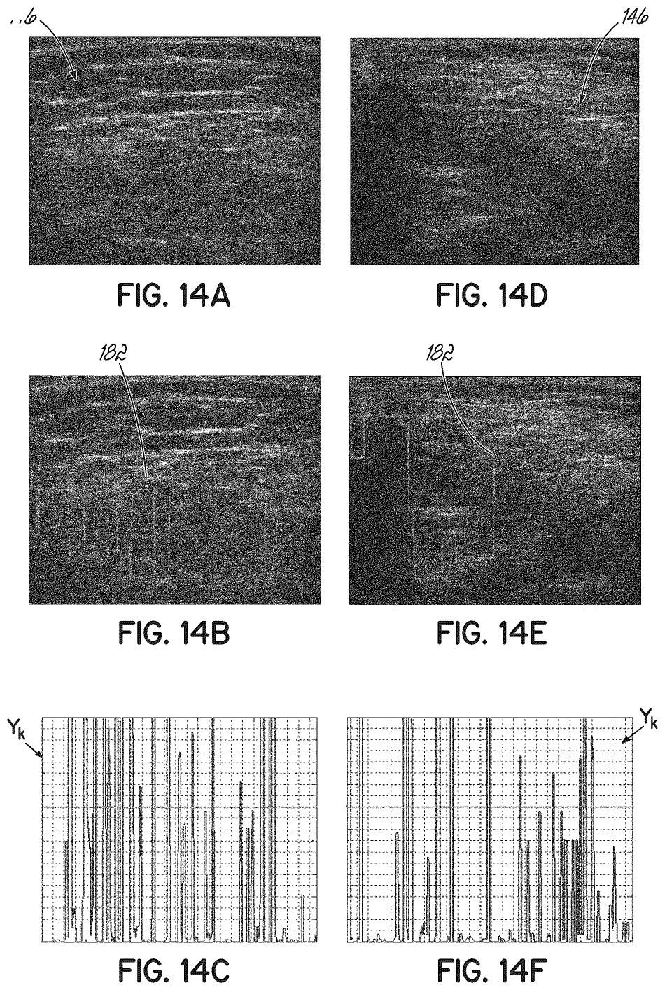

FIGS. 12A-12F and 13A-13F illustrate the resultant bone contour 180 that is selected from those segments of the extracted bone contour that satisfy two conditions: (1) the continuity criteria, having a local standard deviation value below selected standard deviation threshold, and (2) a minimum-length criteria, which avoids piecewise-smooth noise contour segments from being falsely detected as bone contour. In some exemplary embodiments, the length of the standard deviation filter may be set to 3 and the threshold set to 1.16 mm, which may correspond to 30 signal samples. Accordingly, FIGS. 13A and 13D illustrate two exemplary RF signals 142 with the resultant bone contours 180 extracted and filtered from the noise 182 (including isolated outliers and false body echoes), shown in FIGS. 13B and 13E, respectively. FIGS. 13C and 13F respectively illustrate the standard deviation, Yₖ, calculated as provided in Equation 11 above. FIGS. 14A-14F are similar to FIGS. 13A-13F, but include two exemplary RF signals 142 in which no bone tissue was scanned.

With the bone contours isolated from each of the RF signals, the bone contours may now be transformed into a point cloud. For instance, returning now to FIG. 7, the resultant bone contours 180 may then undergo registration with the optical system to construct a bone point cloud 194 representing the surface of at least a portion of each scanned bone (Block 186), which is described herein as a multiple step registration process. In one embodiment, the process is a two-step registration process. The registration step (Block 186) begins by transforming the resultant bone contour 180 from a 2D contour in the ultrasound frame into a 3-D contour in the world frame (Block 188). This transformation is applied to all resultant bone contours 180 extracted from all of the acquired RF signals 142.

To transform the resultant bone contour 180 into the 3-D contour, each detected bone echo 162a undergoes transformation into a 3-D point as follows:

$${d}_{echo} = {n}_{echo} T \text{?} C \text{?} \qquad ( 12 )$$

$${l}_{echo} = {L}_{trans} \frac{{n}_{line}}{{N}_{lines}} {\hat{u}}_{x} \qquad ( 13 )$$

$${P}_{echo}^{OP} = {P}_{trans - origin} + {d}_{echo} {\hat{u}}_{y} + {l}_{echo} {\hat{u}}_{x} \qquad ( 14 )$$

$${P}_{echo}^{W} = {H}_{OP}^{W} {P}_{echo}^{OP} \qquad ( 15 )$$

$$\text{?} \text{indicates text missing or illegible when filed}$$

Where the variables are defined as follows:

| | |
|---|---|
| d_{echo} | depth of the bone echo (cm) |
| n_{echo} | sample index of the detected bone echo |
| Tₛ | RF signal sampling period (sec/sample) |
| Cᵤₛ | speed of ultrasound in soft tissue (154 × 10³ cm/s) |
| l_{echo} | distance from the P_{trans-origin} (FIG. 2) of the transducer |
| | array 68 (FIG. 2) to the current scan line (cm) |
| P_{echo}^{OP} | detected point on the bone surface represented in the local frame |
| nₗᵢₙₑ | index of the scan line containing the bons echo in Ins |

| | |
|---|---|
| Nₗᵢₙₑₛ | number of scan lines in the image |
| P_{echo}^{W} | surface of the bone relative to the world frame |
| H_{OP}^{W} | homogenous transformation between the local |
| | frame and the world frame, as described previously |
| H_{OP}^{W} | dynamically obtained transformation contains the position |
| | and orientation of the optical tracking marker 86 (FIG. 2) |

If so desired, an intermediate registration process may be performed between the resultant bone contour and a B-mode image, if acquired (Block 190). This registration step is performed for visualizing the resultant bone contour 180 with the B-mode image (FIG. 9), which provides visual validation and feedback of the resultant bone contour 180 detection process, in real time, while the user is performing the scan. This visual validation may aid the user in determining whether acquisition is completed (Block 160), as described previously. More specifically, the resultant bone contour 180 is registered with the B-mode image by:

P_{echo}^{I}=(I_{echo}lₓd_{echo}I_{y} (16)

Where lₓ and I_{y} denote the B-mode image resolution (pixels/cm) for the x- and y-axes respectively. P_{echo}^{I} denotes the coordinates of the bone contour point relative to the ultrasound frame.

After the resultant bone contours 180 are transformed and, if desired, registered (Block 190) (FIG. 15), the plurality of point clouds 165 (FIG. 16B) are generated representing the surface of the bone. During the second registration process the plurality of point clouds 165 are integrated into a bone point cloud 194 representing the entire surface of the scanned bone.

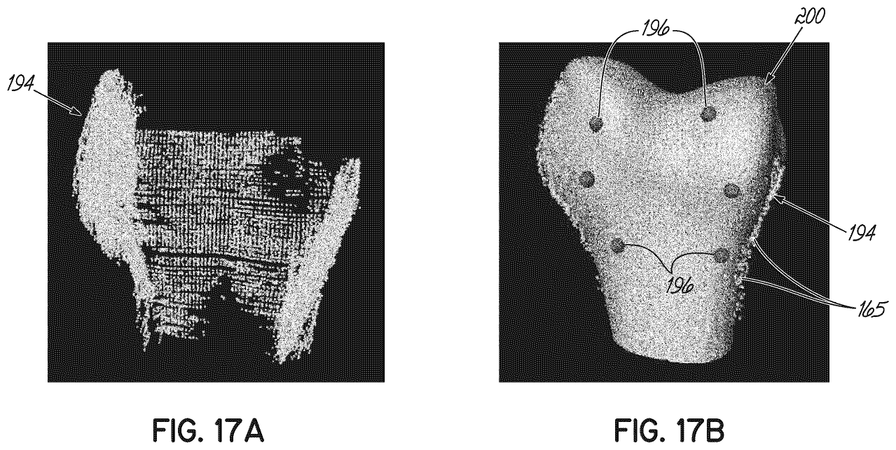

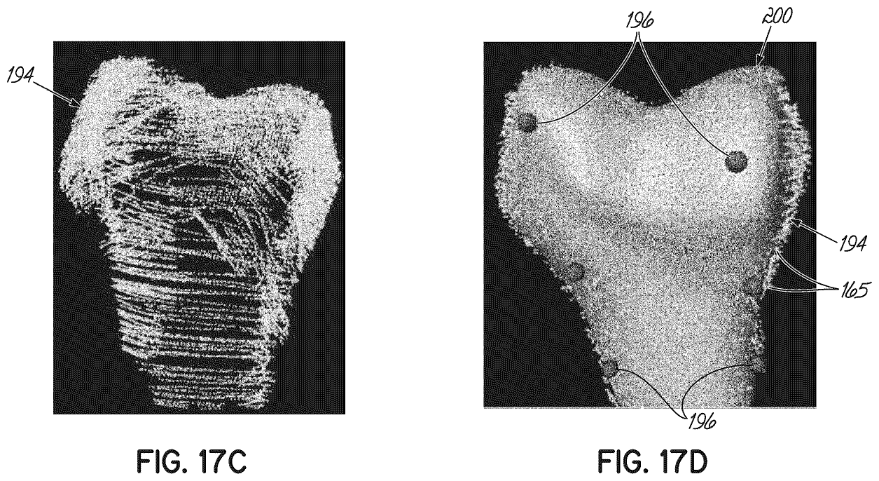

To begin the second registration process, as shown in FIGS. 16A-17D, the plurality of point clouds 194 are initially aligned to a standardized model of the scanned bone, here a model femur 200, for example, by using 4-6 previously specified landmarks 196 (Block 192). More specifically, the user may identify the plurality of landmarks 196 on the model femur 200, which need not be identified with high accuracy. After this initial alignment, an iterative closest point (“ICP”) alignment is performed to more accurately align the standardized model to the plurality of point clouds. If necessary, noise may be removed by thresholding for a distance between a respective point of the plurality of point clouds and the closest vertices in the model femur 200; however, other filtering methods may alternatively be used. For instance, an average distance plus one standard deviation may be used as the threshold. The process is repeated for each point cloud 165 of the plurality for the surface of the scanned bone. The now aligned point clouds 165 are then integrated into a single uniform point cloud 194 that represents the surface of the scanned bone (Block 202).

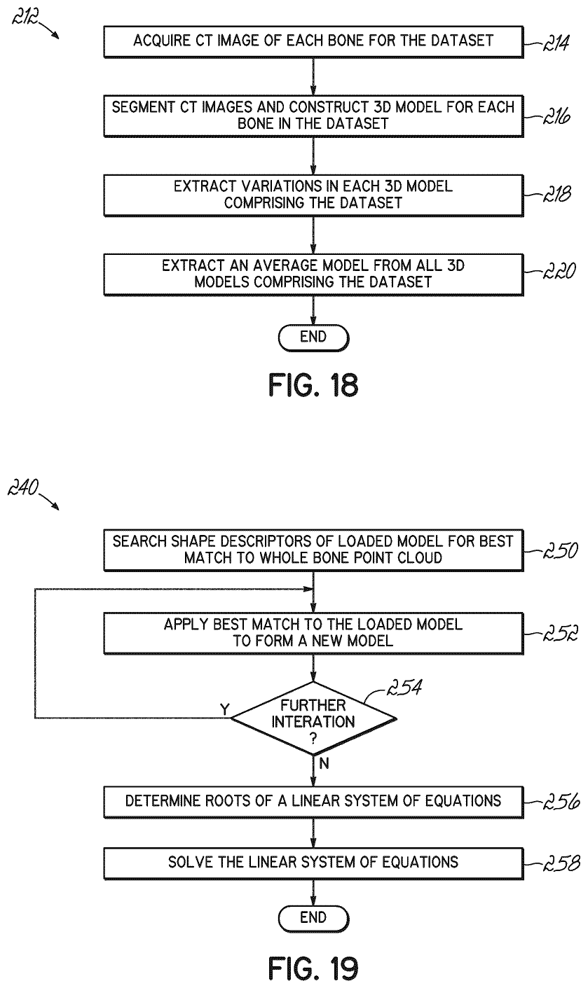

After the point clouds 194 are formed, a bone model may be optimized in accordance with the point clouds 194. That is, the bone point cloud 194 is then used to reconstruct a 3-D patient-specific model of the surface of the scanned bone. The reconstruction begins with a determination of a bone model from which the 3-D patient-specific model is derived (Block 210). The bone model may be a generalized model based on multiple patient bone models and may be selected from a principal component analysis (“PCA”) based statistical bone atlas. One such a priori bone atlas, formed in accordance with the method 212 of FIG. 18, includes a dataset of 400 dry femur and tibia bone pairs, scanned by CT (Block 214) and segmented to create models of each bone (Block 216). The method of building and using one such statistical atlas is described in MAHFOUZ M et al., “Automatic Methods for Characterization of Sexual Dimorphism of Adult Femora: Distal Femur,” Computer Methods in Biomechanics and Biomedical Engineering, 10(6) 2007, the disclosure of which is incorporated herein by reference in its entirety. Each bone model, Mᵢ, (where I∈[1, N], N being the number of models in the dataset) has the same number of vertices, wherein the vertex, Vⱼ, in a select one model corresponds (at the same anatomical location on the bone) to the vertex, Vⱼ, in another one model within the statistical atlas.

PCA is then performed on each model in the dataset to extract the modes of variation of the surface of the bone (Block 218). Each mode of variation is represented by a plurality of eigenvectors resulting from the PCA. The eigenvectors, sometimes called eigenbones, define a vector space of bone morphology variations extracted from the dataset. The PCA may include any one model from the dataset, expressed as a linear combination of the eigenbones. An average model of all of the 3-D models comprising the dataset is extracted (Block 220) and may be defined as:

$${M}_{avg} = \frac{1}{N} {\sum }_{i = 1}^{N} {M}_{i} \qquad ( 17 )$$

$${M}_{i} = {M}_{avg} + {\sum }_{k = 1}^{L} {a}_{ik} {U}_{k} ∀ i ∈ [ 1 , N ] \qquad ( 18 )$$

Where the variables are defined as follows:

| | |
|---|---|
| M_{avg} | is the mean bone of the dataset |
| L | dimensionality of the eigenspace (i.e., the number of |
| | eigenbones) and is equal to N |
| N | number of models in the data |
| Uₖ | k^{th} eigenbone |
| aᵢₖ | k^{th} shape descriptor or eigenbone's coefficient for the i^{th} model |

Furthermore, any new model, M_{new} (i.e., a model not already existing in the dataset), may be approximately represented by new values of the shape descriptors (eigenvectors coefficients) as follows:

$${M}_{new} ≃ {M}_{avg} + {\sum }_{k = 1}^{W} {α}_{k} {U}_{k} \qquad ( 19 )$$

Where the variables are defined as follows:

| | |
|---|---|
| M_{new} | new bone model |
| aₖ | indexed shape descriptors for the new model |
| W | number of principal components to use in the model |
| | approximation, where W ≤ L |

The accuracy of M_{new} is directly proportional to the number of principal components (W) used in approximating the new model and the number of models, L, of the dataset used for the PCA. The residual error or root mean square error (“RMS”) for using the PCA shape descriptors is defined by:

$$RMS = rms [ {M}_{new} - ( {M}_{avg} + {\sum }_{k = 1}^{W} {α}_{k} {U}_{k} ) ] \qquad ( 20 )$$

Therefore, the RMS when comparing any two different models, A and B, having the same number of vertices is defined by:

$$RMS = rms ( A - B ) = \sqrt{\frac{{\sum }_{j = 1}^{m} {‖ {V}_{Aj} - {B}_{Bj} ‖}^{2}}{m}} \qquad ( 21 )$$

Where V_{Aj} is the j^{th} vertex in model A, and similarly, V_{Bj} is the j^{th} vertex in model B.

Returning again to FIG. 7, the average model (“AVERAGE” branch of Block 210) is loaded (Block 230) or a subset model is selected (“SELECTED” branch of Block 210) from the statistical atlas based on demographics that are similar to the patient and loaded (Block 232) for optimization. The bone point cloud 194 is then applied to the loaded model (Block 234) so that the shape descriptors of the loaded model may be changed to create the 3-D patient-specific model. If desired, one or more shape descriptors may be constrained (“YES” branch of Block 254) so that the 3-D patient-specific model will have the same anatomical characteristics as the loaded model. Accordingly, the one or more shape descriptors are set (Block 238). With the constraints set, the loaded model may be deformed (or optimized) (Block 240) into a model that resembles the appropriate bone and not an irregularly, randomly shaped model. If no constraints are desired (“NO” branch of Block 240) and then the loaded model is optimized (Block 240).

Changing the shape descriptors to optimize the loaded model (Block 240) may be carried out by one or more optimization algorithms, guided by a scoring function, to find the values of the principal components coefficients to create the 3-D patient-specific new model and are described with reference to FIG. 19. The illustrated optimization algorithm includes a two-step optimization method of successively-applied algorithms to obtain the 3-D patient-specific model that best fits the bone point cloud 194 as discussed below. Although a two-step method is described, the present invention is not limited to just a two-step optimization method.

The first algorithm may use a numerical method of searching the eigenspace for optimal shape descriptors. More specifically, the first algorithm may be an iterative method that searches the shape descriptors of the loaded model to find a point that best matches the bone point cloud 194 (Block 250). One such iterative method may include, for example, Powell's conjugate gradient descent method with a RMS as the scoring function. The changes are applied to the shape descriptors of the loaded model by the first algorithm to form a new model, M_{new}, (Block 252) defined by Equation 19. The new model, M_{new}, is then compared with the bone point cloud 194 and the residual error, E, calculated to determine whether a further iterative search is required (Block 254). More specifically, given a bone point cloud, Q, having n points therein, and an average model, M_{avg}, with I vertices, there may be a set of closest vertices, V, in the average model, M_{avg} to the bone point cloud, Q.

$${v}_{i} = {arg min}_{{v}_{j} ∈ M} ‖ {v}_{j} - {q}_{i} ‖ ∀ i ∈ [ 1 , n ] , j ∈ [ 1 , l ] \qquad ( 22 )$$

Where vᵢ is the closest point in the set, V, to qᵢ in the bone point cloud, Q. An octree may be used to efficiently search for the closest points in M_{new}. The residual error, E, between the new model, M_{new} and the bone point cloud, Q, is then defined as:

E=∥V−Q∥² (23)

With sufficiently high residual error (“YES” branch of Block 254), the method returns to further search the shape descriptors (Block 250). If the residual error is low (“NO” branch of Block 254), then the method proceeds.

The second algorithm of the two-step method refines the new model derived from the first algorithm by transforming the new model into a linear system of equations in the shape descriptors. The linear system is easily solved by linear system equation, implementing conventional solving techniques, which provide the 3-D patient-specific shape descriptors.

In continuing with FIG. 19, and to transform the new model into the linear system, the roots of the linear system must be determined (Block 256). More specifically, the first partial derivatives of the residual error, E, with respect to the shape descriptors, αₖ, are equal to zero. The error function, Equation 23, may be expressed in terms of the vertices, vᵢ, of the set, V, and the points, pᵢ, of the point cloud, Q:

$$E = {\sum }_{i = 1}^{m} {‖ {v}_{i} - {q}_{i} ‖}^{2} \qquad ( 24 )$$

And may also be expressed in terms of the new model's shape descriptors as:

$$E = {‖ ( {V}_{avg} + {\sum }_{k = 1}^{W} {α}_{k} {U}_{k}^{′} ) - Q ‖}^{2} \qquad ( 25 )$$

Where V_{avg} is the set of vertices from the loaded model's vertices, which corresponds to the vertices set, V, that contains the closest vertices in the new model, M_{new}, that is being morphed to fit the bone point cloud, Q. Uₖ′ is a reduced version of the eigenbone, Uₖ, containing only the set of vertices corresponding to the vertices set, V.

Combining Equations 24 and 25, E maybe expressed as:

$$E = {\sum }_{i = 1}^{m} {‖ ( {v}_{avg , i} + {\sum }_{k = 1}^{W} {α}_{k} {u}_{k , i}^{′} ) - {q}_{i} ‖}^{2} \qquad ( 26 )$$

Where v_{avg,I} is the i^{th} vertex of V_{avg}. Similarly, u_{k′,I} is the i^{th} vertex of the reduced eigenbone, Uₖ′.

The error function may be expanded as:
¾

E=Σᵢ₌₁^{m}[(x_{avg,i}+Σᵢ₌₃^{W}aₖx_{u′,i,i}−x_{q,i})²+(y_{avg,i}+Σᵢ₌₁^{W}aₖy_{u′,i,i}−y_{q,i})²+(z_{avg,i}+Σᵢ₌₃^{W}aᵢz_{u′,i,i}−z_{q,i})²] (27)

Where x_{avg,I} is the x-coordinate of the i^{th} vertex of the average model, x_{k,I} is the x-coordinate of the i^{th} vertex of the k^{th} eigenbone, and x_{Q,I} is the x-coordinate of the i^{th} point of the point cloud, Q. Similar arguments are applied to the y- and z-coordinates. Calculating the partial derivative of E with respect to each shape descriptor, αₖ, yields:

$$\frac{\partial E}{\partial {α}_{k}} = 0 ∀ k ∈ [ 1 , W ] \qquad ( 28 )$$

$$\frac{\partial E}{\partial {α}_{k}} = {\sum }_{i = 1}^{m} [ 2 ( {x}_{avg , i} + {\sum }_{i = 1}^{W} {α}_{i} {x}_{{u}^{′}} \text{?} - x \text{?} ) {x}_{k , i} + 1 ( {y}_{avg , i} + {\sum }_{i = 1}^{W} {α}_{i} y \text{?} - y \text{?} ) y \text{?} + 2 ( {x}_{avg , i} + {\sum }_{i = 1}^{W} {α}_{k} {z}_{{u}^{′}} \text{?} - z \text{?} ) {z}_{k , i} ] = 0 ∀ k ∈ [ 1 , W ] \qquad ( 29 )$$

$$\text{?} \text{indicates text missing or illegible when filed}$$

Recombining the coordinate values into vectors yields:

$$\frac{\partial E}{\partial {α}_{k}} = {\sum }_{i = 1}^{m} [ ( {v}_{avg , i} , u \text{?} ) + ( {\sum }_{i = 1}^{W} {α}_{i} u \text{?} ) \text{?} {u}_{k , i}^{′} - {q}_{i} \text{?} {u}_{k , i}^{′} ] = 0 ∀ k ∈ [ 1 , W ] \qquad ( 30 )$$

$$\text{?} \text{indicates text missing or illegible when filed}$$

And with rearrangement:

$${\sum }_{i = 1}^{m} ( {\sum }_{i = 1}^{w} {α}_{i} ( {u}^{′} \text{?} {u}_{k , i}^{′} ) ) = {\sum }_{i = 1}^{m} [ {q}_{i} \text{?} {u}_{k , i}^{′} - ( {v}_{avg , i} \text{?} {u}_{k , i}^{′} ) ] \qquad ( 31 )$$

$$\text{?} \text{indicates text missing or illegible when filed}$$

Reformulating Equation 31 into a matrix form provides a linear system of equations in the form of Ax=B:

$${\sum }_{i = 1}^{m} [ \begin{matrix}{u}_{2 , i}^{′} \text{?} {u}_{1 , i}^{′} & {u}_{2 , i}^{′} \text{?} {u}_{1 , i}^{′} & … & … & {u}_{W , i}^{′} \text{?} {u}_{1 , i}^{′} \\ {u}_{1 , i}^{′} \text{?} {u}_{2 , i}^{′} & {u}_{2 , i}^{′} \text{?} {u}_{2 , i}^{′} & … & … & {u}_{W , i}^{′} \text{?} {u}_{2 , i}^{′} \\ ⋮ & ⋮ & ⋱ & ⋱ & ⋮ \\ ⋮ & ⋮ & ⋱ & ⋱ & ⋮ \\ {u}_{1 , i}^{′} \text{?} {u}_{W , i}^{′} & {u}_{2 , i}^{′} \text{?} {u}_{W , i}^{′} & … & … & {u}_{W , i}^{′} \text{?} {u}_{W , i}^{′}\end{matrix} ] [ \begin{matrix}\begin{matrix}\begin{matrix}\begin{matrix}{a}_{1} \\ {a}_{2}\end{matrix} \\ ⋮\end{matrix} \\ ⋮\end{matrix} \\ {a}_{W}\end{matrix} ] = {\sum }_{i = 1}^{m} [ \begin{matrix}\begin{matrix}\begin{matrix}\begin{matrix}( {q}_{i} - {v}_{avg , i} ) \text{?} {u}_{1 , i}^{′} ) \\ ( {q}_{i} - {v}_{avg , i} ) \text{?} {u}_{2 , i}^{′} )\end{matrix} \\ ⋮\end{matrix} \\ ⋮\end{matrix} \\ ( {q}_{i} - {v}_{avg , i} ) \text{?} {u}_{W , i}^{′} )\end{matrix} ] \qquad ( 32 )$$

$$\text{?} \text{indicates text missing or illegible when filed}$$

The linear system of equations may be solved using any number of known methods, for instance, singular value decomposition (Block 258).

In one embodiment, the mahalanobis distance is omitted because the bone point clouds are dense, thus providing a constraining force on the model deformation. Therefore the constraining function of the mahalanobis distance may not be needed, but rather was avoided to provide the model deformation with more freedom to generate a new model that best fit the bone point cloud.

An ultrasound procedure in accordance with the embodiments of the present invention may, for example, generate approximately 5000 ultrasound images. The generated 3-D patient-specific models (Block 260, FIG. 7), when compared against CT-based segmented models, yielded an average error of approximately 2 mm.

The solution to the linear set of equations provides a description of the patient-specific 3-D model, derived from an average, or select model, from the statistical atlas, and optimized in accordance with the point cloud transformed from a bone contour that was isolated from a plurality of RF signals. The solution may be applied to the average model to display a patient-specific 3-D bone model for aiding in pre-operative planning, mapping out injection points, planning a physical therapy regiment, or other diagnostic and/or treatment-based procedure that involves a portion of the musculoskeletal system.

Cartilage 3-D models may be reconstructed a method that is similar to that which was outlined above for bone. During contour extraction, the contour of the cartilage is more difficult to detect than bone. Probabilistic modeling (Block 171) is used to process the raw RF signal to more easily identify cartilage, and SVM aids in detection of cartilage boundaries (Block 173) based on MRI training sets. A cartilage statistical atlas is formed by a method that may be similar to what was described for bone; however, as indicated previously, MRI is used rather than the CT (which was the case for bone). The segmentation (Block 216), variation extraction (Block 218) and base model morphing (Block 240) (FIG. 19) are processed to produce a reconstructed cartilage model in the same manner as a bone model is reconstructed. The cartilage model may be displayed alone, or in conjunction with the 3D patient-specific bone model.

Referring now to FIGS. 20-27, and in accordance with another embodiment of the invention, an additional method of extracting bone contours and generating point clouds from raw RF ultrasound signals is described. Referring now to FIG. 20, the ultrasound instrument 50 is shown in more detail with the electromagnetic tracking system 87, and the computer 54. The ultrasound instrument 50 may include an ultrasound transceiver 356 operatively coupled to the ultrasound probe 60 by a cable 66, and a controller 360. The ultrasound transceiver 356 generates drive signals that excite the ultrasound probe 60 so that the ultrasound probe 60 generates ultrasound signals 362 that can be transmitted into the patient. In an embodiment of the invention, the ultrasound signals 362 comprise bursts or pulses of ultrasound energy suitable for generating ultrasound images. The ultrasound probe 60 may also include the tracking marker 86, shown here as an electromagnetic tracking marker 86.

Reflected ultrasound signals, or echoes 364, are received by the ultrasound probe 60 and converted into RF signals that are transmitted to the transceiver 356. Each RF signal may be generated by a plurality of echoes 364, which may be isolated, partially overlapping, or fully overlapping. Each of the plurality of echoes 364 originates from a reflection of at least a portion of the ultrasound energy at an interface between two tissues having different densities, and represents a pulse-echo mode ultrasound signal. One type of pulse-echo mode ultrasound signal is known as an “A-mode” scan signal. The controller 360 converts the RF signals into a form suitable for transmission to the computer 54, such as by digitizing, amplifying, or otherwise processing the signals, and transmits the processed RF signals to the computer 54 via the I/O interface 85. In an embodiment of the invention, the signals transmitted to the computer 54 may be raw RF signals representing the echoes 364 received by the ultrasound probe 60.

The electromagnetic tracking system 87 includes an electromagnetic transceiver unit 328 and an electromagnetic tracking system controller 366. The transceiver unit 328 may include one or more antennas 368, and transmits a first electromagnetic signal 370. The first electromagnetic signal 370 excites the tracking marker 86, which responds by transmitting a second electromagnetic signal 372 that is received by the transceiver unit 328. The tracking system controller 366 may then determine a relative position of the tracking marker 86 based on the received second electromagnetic signal 372. The tracking system controller 366 may then transmit tracking element position data to the computer 54 via I/O interface 85.

Referring now to FIG. 21, a flow chart 380 illustrates an alternative embodiment of the invention in which the acquired scan data is used to reconstruct patient-specific bone models. The patient-specific bone models may be generated from raw RF signals that are used directly to automatically extract bone contours from ultrasound scans. Specifically, these embodiments of the invention include additional methods of bone/cartilage contour detection, point cloud, and 3-D model reconstruction from ultrasound RF signal data. The ultrasound signal processing of these alternative embodiments optimizes scan reconstruction through a multi-tier signal processing model. The processing algorithm is broken down into multiple models, which are separated into different tiers. Each tier performs specific optimization or estimation to the data. The primary functions of the tiers include, but are not limited to, raw signal data optimization for features detection and estimation, scan-line features detection, global feature estimations, updates, and smoothing. The tiers operate within the framework of Bayesian inference model. The features and properties of the algorithm inputs are determined by mathematical and physical models within the tier. One example of this processing model implementation is a three-tier processing system, which is described below.

The first tier of the three-tier system optimizes the raw signal data and estimates the envelope of the feature vectors. The second tier estimates the features detected from each of the scan lines from the first tier, and constructs the parametric model for Bayesian smoothing. The third tier estimates the features extracted from the second tier to further estimate the three-dimensional features in real-time using a Bayesian inference method.

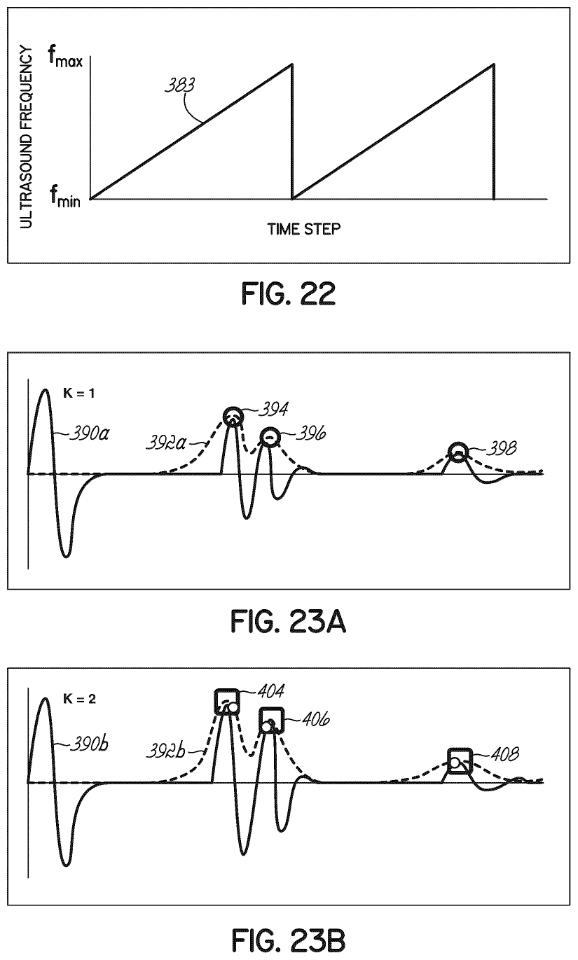

In block 382, raw RF signal data representing ultrasound echoes 364 detected by the ultrasound probe 60 is received by the program code 83 and processed by a first layer of filtering for feature detection. The feature vectors detected include bone, fat tissues, soft tissues, and muscles. The optimal outputs are envelopes of these features detected from the filter. There are two fundamental aspects of this design. The first aspect relates to the ultrasound probe 60 and the ultrasound controller firmware. In conventional ultrasound machines, the transmitted ultrasound signals 362 are generated at a fixed frequency during scanning. However, it has been determined that different ultrasound signal frequencies reveal different soft tissue features when used to scan the patient. Thus, in an embodiment of the invention, the frequency of the transmitted ultrasound signal 362 changes with respect to time using a predetermined excitation function. One exemplary excitation function is a linear ramping sweep function 383, which is illustrated in FIG. 22.

The second aspect is to utilize data collected from multiple scans to support a Bayesian model for estimation, correction, and optimization. Two exemplary filter classes are illustrated in FIG. 21, either of which may be used to support the algorithm. In decision block 384, the program code 83 selects a feature detection model that determines the class of filter through which to process the RF signal data. If the data is to be processed by a linear filter, the application proceeds to block 386. In block 386, the imaging program code 83 selects a linear class of filter, such as a linear Gaussian model, or non-linear Gaussian model with linearization methods, based on the Kalman filter family. The operation of this linear class of filters is illustrated in more detail by FIGS. 23A and 23B, which outline the basic operation of the Kalman filter, upon which other extensions of the filter are built.

In block 388, an optimal time delay is estimated using a Kalman class filter to identify peaks in the amplitude or envelope of the RF signal. Referring now to FIG. 23A, at time k=1, the filter is initialized by setting the ultrasound frequency fₖ=f₁. The received echo or RF signal (s_{obs}) is represented by plot line 390a, while the signal envelope is represented by plot line 392a. The peak data matrix (p_{k,fk}), which contains the locations of the RF signal peaks, may be calculated by:

p_{k,fk}=E(s_{obs}) (33)

where E is an envelope detection and extraction function. The peak data matrix (p_{k,fk}) thereby comprises a plurality of points representing the signal envelope 392, and can be used to predict the locations of envelope peaks 394, 396, 398 produced by frequency fₖ₊₁ using the following equation:

p_{est,fk+1}=H(p_{k,fk+1}) (34)

where H is the estimation function.

Referring now to FIG. 23B, at time k=2, the filter enters a recursive portion of the algorithm. The frequency of the transmitted ultrasound signal 362 is increased so that fₖ=f₂, and a new RF signal is received (s_{obs,fk}), as represented by plot line 390b. The new RF signal 390b also generates a new signal envelope 392b. A peak data matrix is calculated (p_{k,fk}) for the new signal envelope 392b, which identifies another set of peaks 404, 406, 408. The error of the prediction is computed by:

ε=p_{est,fk-1}−p_{k,fk} (35)

and the error correction (Kalman) gain (Kₖ) is computed by:

Kₖ=Pₖ⁻H^{T}(HPₖ⁻H^{T}+R) (36)

where Pₖ_{^{−}} is the error covariance matrix, and R is the covariance matrix of the measurement noise. The equation for estimating the peak data matrix for the next cycle becomes:

p_{est,k+1}=p_{k,fk}+Kₖ(ε) (37)

and the error covariance is updated by:

Pₖ=(1−KₖH)Pₖ⁻ (38)

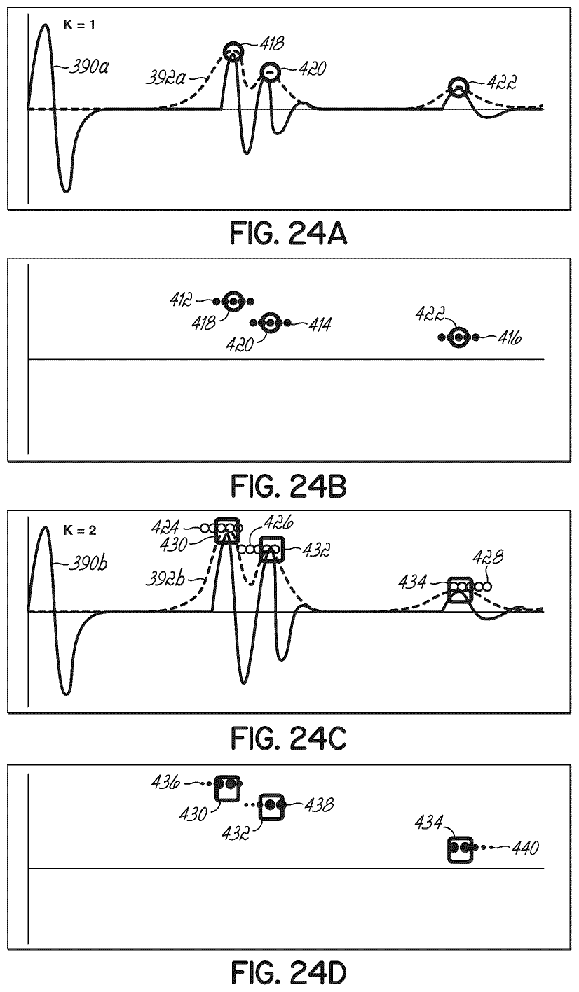

If the second class of filter is to be used, the program code 83 proceeds to block 410 rather than block 386 of flow chart 380, and selects a non-linear, non-Gaussian model that follows the recursive Bayesian filter approach. In the illustrated embodiment, a Sequential Monte Carlo method, or particles filter is shown as an exemplary implementation of the recursive Bayesian filter. In block 412, the program code 83 estimates an optimal time delay using the particles filter, to identify signal envelope peaks. An example of a particles filter is illustrated in FIGS. 24A and 24B. In principle, the particle filter generates a set of N equally weighted particles (p_{k,fk}) 412, 414, 416 around each envelope peak 418, 420, 422 of the peak data matrix detected during the initialization. The sets of equally weighted particles are based on an arbitrary statistical density (p), which is approximated by:

ρ_{k,fk}^{i:1-N}˜ [symbol] (p_{k,fk}|s_{obs}) (39)

These particles 41 1, 414, 416 predict the peak locations at fₖ₊₁ via the following equation:

p_{est,k+1}^{i:1-N}=H(ρ_{k,fk}^{i:1-N}) (40)

where H is the estimation function.

Referring now to FIGS. 24C and 24D, at time k=2, a new peak data matrix (p_{k,fk}) is calculated when the RF signal 90b (s_{obs}) becomes available, and new sets of estimation particles 424, 426, 428 are made around each peak 430, 432, 434 for (fₖ=f₂). The estimation particles of sets 41 1, 414, 416 from time k=1 are compared with the observed data obtained at time k=2, and an error is determined using the following equation:

εₖ^{i:1-N}=p_{est,k+1}^{i:1-N}−p_{k,fk} (41)

The normalized importance weights of the particles of particle sets 424, 426, 428 are evaluated as:

$$\text{?} \qquad ( 42 )$$

$$\text{?} \text{indicates text missing or illegible when filed}$$

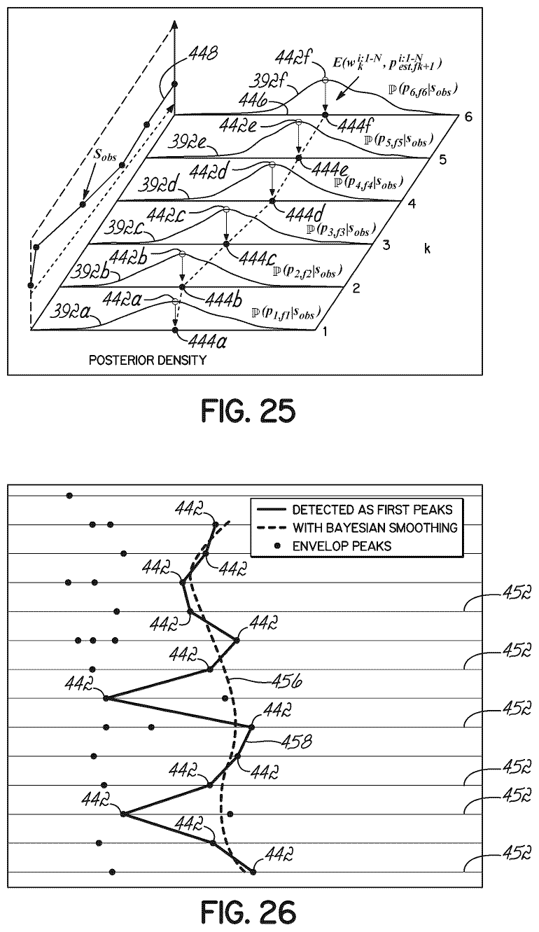

which produces weighted particle sets 436, 438, 440. This step is known as importance sampling where the algorithm approximates the true probability density of the system. An example of importance sampling is shown in FIG. 25, which illustrates a series of signal envelopes 392a-392f for times k=1-6. Each signal envelope 392a-392f includes a peak 442a-442f and a projection 444a-444f of the peak 442a-442f onto a scan-line time scale 446 that indicates the echo return time. These projections 444a-444f may, in turn, be plotted as a contour 448 that represents an estimated location of a tissue density transition or surface. In any case, the expectation of the peak data matrix can then be calculated based on the importance weight and the particles' estimate:

p_{k,fk}= [symbol] (wₖ^{i:1-N},p_{est,fk+1}^{i:2-N} (44)

In addition, particle maintenance may be required to avoid particle degeneracy, which refers to a result in which the weight is concentrated onto a few particles over time. Particle re-sampling can be used by replacing degenerated particles with new particles sampled from the posterior density:

[symbol]

(p_{est,fk+1}^{i:2-N}) (44)

Referring now to FIG. 26, once the envelope peaks have been identified, the program code 83 proceeds to block 450 and applies Bayesian smoothing to the envelope peaks 442 in temporally adjacent scan lines 452 before proceeding to block 454 and extracting 2-D features from the resulting smoothed contour line 456. This second layer of the filter thus applies a Bayesian technique to smooth the detected features on a two-dimensional level. Conventional peak detection methods have a limitation in that the envelope peaks 442 across different scan lines are not statistically weighted. Thus, only the peaks 442 with the highest power are detected for reconstruction. This may result in an erroneous contour, as illustrated by contour line 458, which connects the envelope peaks 442 having the highest amplitude. Therefore, signal artifacts or improper amplitude compensation by gain control circuits in the RF signal path may obfuscate the signal envelope containing the feature of interest by distorting envelope peak amplitude. Hence, the goal of filtering in the second layer is to correlate signals from different scan lines to form a matrix that determines or identifies two-dimensional features.

This is achieved in embodiments of the invention by Bayesian model smoothing, which produces the exemplary smoothed contour line 456. The principle is to examine the signal envelope data retrospectively and attempt to reconstruct the previous state. The primarily difference between the Bayesian estimator and the smoother is that the estimator propagates the states forward in each recursive scan, while the smoother operates in the reverse direction. The initial state of the smoother begins at the last measurement and propagates backward. A common implementation of a smoother is the Rauch-Tung-Striebel (RTS) smoother. The feature embedded in the ultrasound signal is initialized based on a priori knowledge of the scan, which may include ultrasound transducer position data received from the electromagnetic tracking system 87. Sequential features are then estimated and updated in the ultrasound scan line with the RTS smoother.

In an embodiment of the invention, the ultrasound probe 60 is instrumented with the electromagnetic or optical tracking marker 86 so that the motion of the ultrasound probe 60 is accurately known. This tracking data 460 is provided to the program code 83 in block 462, and is needed to determine the position of the ultrasound probe 60 since the motion of the ultrasound probe 60 is arbitrary relative to the patient's joint. As scans are acquired by the ultrasound probe 60, the system estimates 3-D features of the joint, such as the shape of the bone and soft tissue. A tracking problem of this type can be viewed as a probability inference problem in which the objective is to calculate the most likely value of a state vector Xi given a sequence of measurements yi, which are the acquired scans. In an embodiment of the invention, the state vector Xi is the position of the ultrasound probe 60 with respect to some fixed known coordinate system or “world frame” (such as the ultrasound machine at time k=0), as well as the modes of the bone deformation. Two main steps in tracking are:
(1) Prediction—The states of the system at k=i can be predicted given all the measurements up through time k=i−1. To do this, the conditional probability P(Xᵢ|y₀, y₁, . . . , yᵢ₋₁), called the prior distribution, must be computed. If it is assumed that the process is a first order Markov process, this can be computed by integrating P(Xᵢ|Xᵢ₋₁)P(Xᵢ|y₀, y₁, . . . , yᵢ₋₁) over all Xᵢ₋₁.
and
(2) Correction—Given a new measurement yᵢ, correct the estimate of the state. To do this, the probability P(Xᵢ|y₀, y₁, . . . , yᵢ), called the posterior distribution, must be computed.

A system dynamics model relates the previous state Xᵢ₋₁ to the new state X, via the transitional distribution P(Xᵢ|Xᵢ₋₁), which is a model of how the state is expected to evolve with time. In an embodiment of the invention, Xᵢ are the 3-D feature estimates calculated from the Bayesian contour estimation performed during tier 2 filtering, and the transformation information contains the translations and rotations of the data obtained from the tracking system 87. With joint imaging, the optimal density or features are not expected to change over time, because the position of the bone is fixed in space and the shape of the bone scanned does not change. Hence, the transitional distribution does not alter the model states.

A measurement model relates the state to a predicted measurement, y=f(X). Since there is uncertainty in the measurement, this relationship is generally expressed in terms of the conditional probability P(yᵢ|Xᵢ), also called the likelihood function. In an embodiment of the invention, the RF signal and a priori feature position and shape are related by an Anisotropic Iterative Closest Point (AICP) method.

To estimate position and shape of the feature, the program code 83 proceeds to block 464. At block 464, the program code 83 performs an AICP method that searches for the closest point between the two datasets iteratively to establish a correspondence by the anisotropic weighted distance that is calculated from the local error covariance of both datasets. The correspondence is then used to calculate a rigid transformation that is determined iteratively by minimizing the error until convergence. The 3-D features can then be predicted based on the received RF signal and the a priori feature position and shape. By calculating the residual error between the predicted 3-D feature and the RF signal data, the a priori position and shape of the feature are updated and corrected in each recursion. Using Bayes' rule, the posterior distribution can be computed based on measurements from the raw RF signal.

If both the dynamic model and the measurement model are linear with additive Gaussian noise, then the conditional probability distributions are normal distributions. In particular, P(Xᵢ|y₀, y₁, . . . , yᵢ) is unimodal and Gaussian, and thus can be represented using the mean and covariance of the predicted measurements. Unfortunately, the measurement model is not linear and the likelihood function P(yᵢ|Xᵢ) is not Gaussian. One way to deal with this is to linearize the model about the local estimate, and assume that the distributions are locally Gaussian.

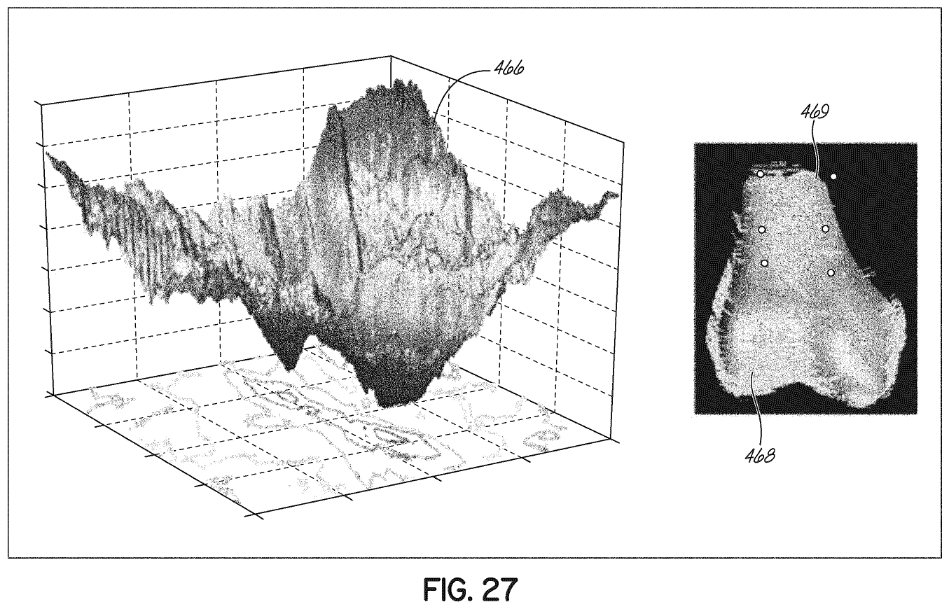

Referring to FIG. 27, a surface 466 representing an exemplary probability distribution associated with a point cloud 468 of a scanned bone 469 illustrates that the probability distribution for the measurement model is not Gaussian, and has many peaks. This suggests multiple hidden states are presented in the model. The posterior probability P(Xᵢ|y₀, y₁, . . . , yᵢ) would also have multiple peaks. The problem would be worse if the state included shape parameters as well as position. A linear tracking filter such as the Kalman filter (or its nonlinear extension, the Extended Kalman filter) cannot deal with non-linear and non-Gaussian system with multi-peaks distribution, which may converge upon the wrong solution.

Instead of treating the probability distributions as Gaussian, a statistical inference can be performed using a Monte Carlo sampling of the states. The optimal position and shape of the feature are thereby estimated through the posterior density, which is determined from sequential data obtained from the RF signals. For recursive Bayesian estimation, one exemplary implementation is particle filtering, which has been found to be useful in dealing in applications where the state vector is complex and the data contain a great deal of clutter, such as tracking objects in image sequences. The basic idea is to represent the posterior probability by a set of independent and identically distributed weighted samplings of the states, or particles. Given enough samples, even very complex probability distributions can be represented. As measurements are taken, the importance weights of the particles are adjusted using the likelihood model, using the equation wⱼ′=P(yᵢ|Xᵢ) wⱼ, where wⱼ is the weight of the j-th particle. This is known as importance sampling.

The principal advantage of this method is that the method can approximate the true probability distribution of the system, which cannot be determined directly, by approximating a finite set of particles from a distribution from which samples can be drawn. As measurements are obtained, the algorithm adjusts the particle weights to minimize the error between the prediction and observation states. With enough particles and iterations, the posterior distribution will approach the true density of the system. A plurality of bone or other anatomical feature surface contour lines is thereby generated that can be used to generate 3-D images and models of the joint or anatomical feature. These models, in turn, may be used to facilitate medical procedures, such as joint injections, by allowing the joint or other anatomical feature to be visualized in real time during the procedure using an ultrasound scan.

International Publication Number WO2014/121244, published Aug. 7, 2014, of International Application No. PCT/US2014/014526, filed Feb. 4, 2014, describes exemplary 3-D reconstruction of joints using ultrasound and is incorporated by reference herein in its entirety.

3-D Reconstruction Using Multiple Imaging Modalities

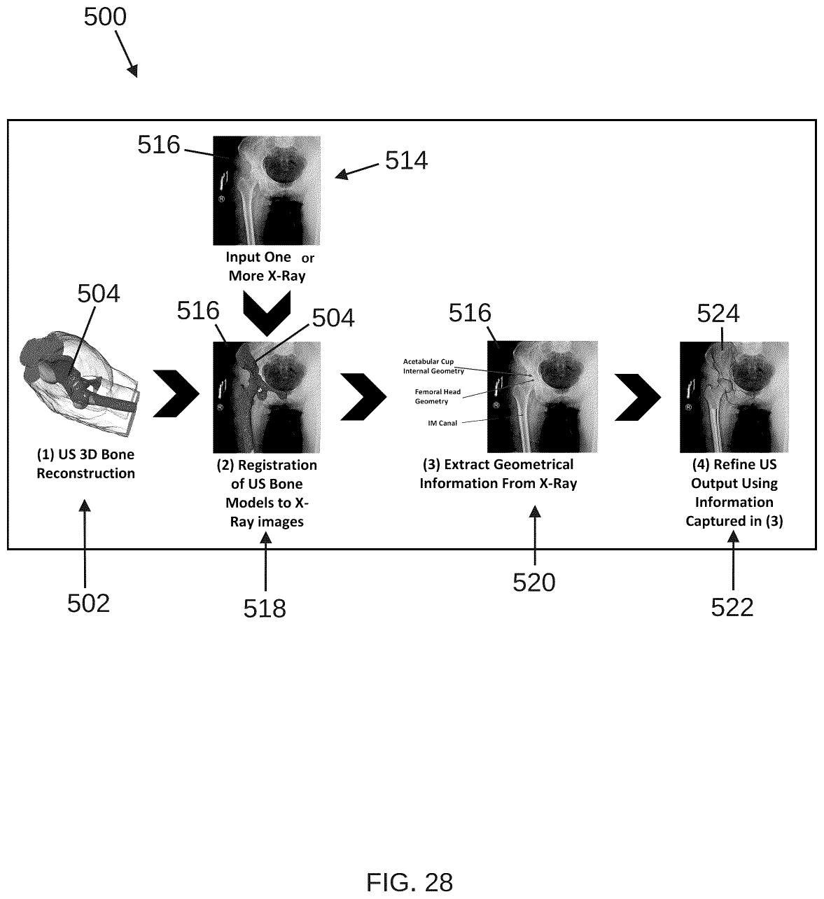

FIG. 28 is a flow diagram of an example method 500 of generating a virtual 3-D model of an anatomical structure using multiple imaging modalities, according to at least some aspects of the present disclosure. The method 500 is described in connection with creation of the 3-D virtual model of a hip joint comprising a pelvis and a femur; however, it will be understood that various aspects of the method may be utilized in connection with modeling various other anatomical structures, including individual bones (e.g., tibia only, patella only, scapula only, humerus only, femur only, and/or pelvis only, etc.), joints comprising multiple bones (hips, knees, shoulder, ankles, etc.), as well as soft tissues (cartilage, ligaments, tendons, etc.) separately or in connection with one or more other anatomical structures. Further, it will be understood that the operations associated with this method may be performed in connection with one or more anatomical structures, generally simultaneously or sequentially. For example, as described below, the pelvis and femur may be modeled together in a coordinated process. In alternative sequentially configured embodiments, one or more anatomical structures may be modeled accordingly to the method, then, subsequently, the method may be performed on one or more other anatomical structures.

The method 500 may include an operation 502, including obtaining a preliminary virtual 3-D bone model 504 of one or more bones. For example, an ultrasound scanning and 3-D reconstruction process described above in the 3-D Reconstruction of Joints Using Ultrasound section may be utilized to generate the preliminary virtual 3-D bone model 504. Although the 3-D bone model 504 may comprise the final output of the 3-D reconstruction processes described above, in the context of this method 500, the bone model 504 may be “preliminary” because it may be subject to refinement in later operations.

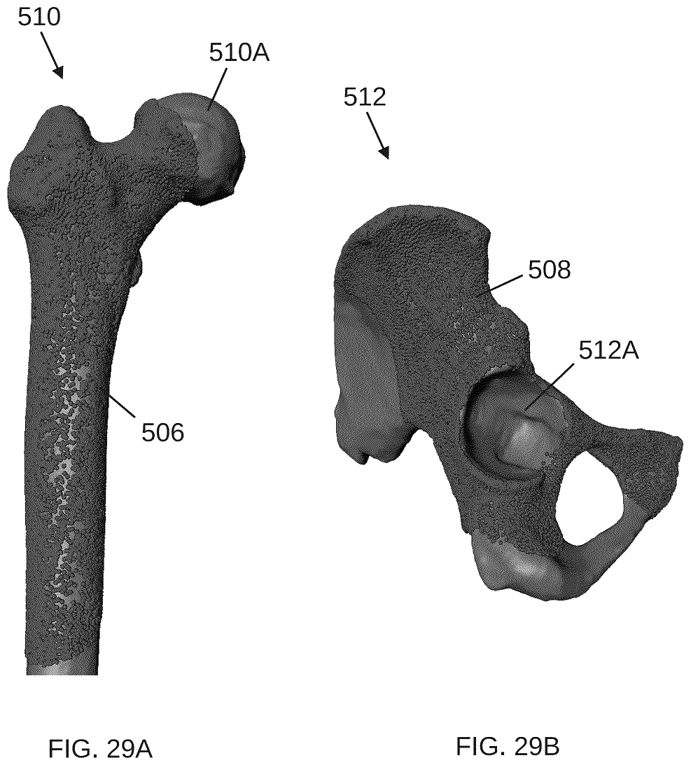

FIGS. 29A and 29B are isometric views of example ultrasound point clouds 506, 508 of a femur 510 and a pelvis 512, respectively, all according to at least some aspects of the present disclosure. Referring to FIGS. 28, 29A, and 29B, in the illustrated embodiment, obtaining the preliminary 3-D bone model 504 may include obtaining one or more ultrasound point clouds, such as point clouds 506, 508, of one or more bones 510, 512. The ultrasound point cloud 506 of the femur 510 may include the femoral neck. The ultrasound point cloud 508 of the pelvis 512 may include at least a portion of the acetabulum (e.g., the rim). Generally, in some example embodiments, ultrasound data may include one or more bones and/or one or more tissues other than bones, such as ligaments, muscles, fat tissues, tendons, and/or cartilage (collectively, “soft tissues”). For example, embodiments pertaining to a hip may include ultrasound imaging of ligaments such as the ischiofemoral ligament, the iliofemoral ligament, and/or the transverse ligament. Generally, the ultrasound data may be used to reconstruct virtual bone and/or soft tissue 3-D models.

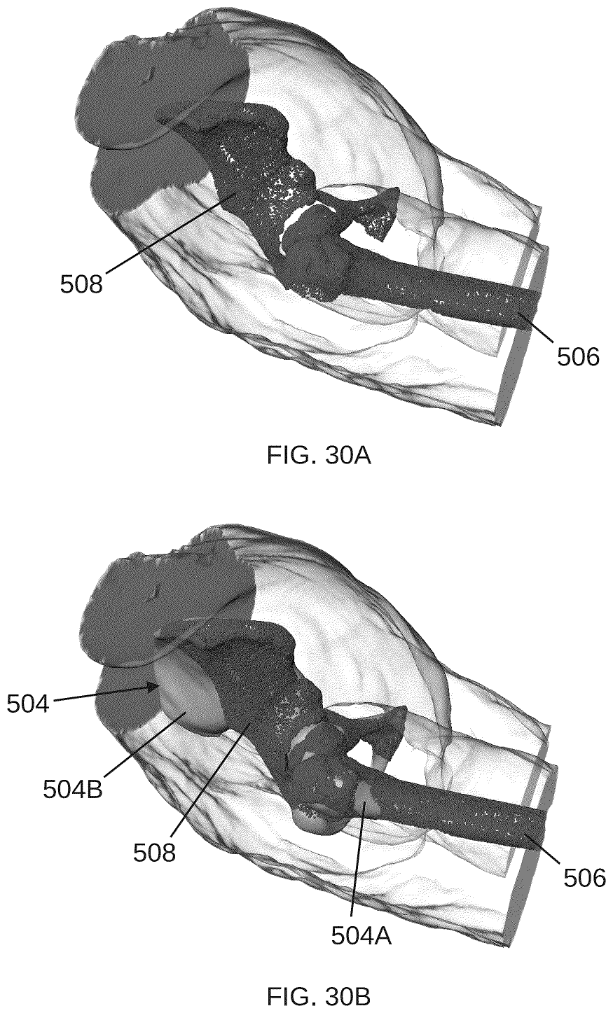

FIG. 30A is an isometric view of the point clouds 506, 508 arranged as obtained by ultrasound scanning a patient's hip joint and FIG. 30B is an isometric view of the point clouds 506, 508 overlaid on the preliminary 3-D model 504, all in accordance with at least some aspects of the present disclosure. In particular, in this embodiment, the point cloud 506 of the femur 510 may be associated with a preliminary 3-D model of the femur 504A and/or the point cloud 508 of the pelvis 512 may be associated with a preliminary 3-D model of the pelvis 504B. In this embodiment, the preliminary 3-D model 504 of FIG. 28 comprises both the preliminary 3-D model of the femur 504A and the preliminary 3-D model of the pelvis 504B.

Referring to FIGS. 29A, 29B, 30A, 30B, the point clouds 506, 508 may not include at least some portions of the respective bones 510, 512. For example, for the femur 510, portions of the femoral head 510A may not be included in the point cloud 506, such as, without limitation, because the femoral head 510A may be at least partially occluded from ultrasound imaging by portions of the pelvis 512. As another example, for the pelvis 512, portions of the acetabular cup 512A may not be included in the point cloud 508, such as, without limitation, because the acetabular cup 512A may be at least partially occluded from ultrasound imaging by portions of the femur 510. In the context of some anatomies, the bones and/or joints may be repositioned to increase exposure of the anatomy to ultrasound imaging. For example, as described above, a knee joint may be positioned in different degrees of flexion to facilitate ultrasound imaging of portions that may be occluded in other degrees of flexion. Similarly, in the context of a hip, the femoral head and the acetabular cup or ring may be scanned in multiple poses, such as by dynamic scanning. In addition to increasing the portions of the surfaces of the anatomical structures that are included in the point clouds, scanning in multiple poses may facilitate determination of a preoperative range of motion for the joint.

While the above examples pertain to exterior surfaces of bones that were occluded from ultrasound imaging by other bones, in some embodiments, significant internal features of some bones may not be included in the point clouds 506, 508. For example, in the context of hip replacement surgery, the intermedullary canal of the femur may be an important anatomical feature for selecting, sizing, and/or installing the femoral implant, particularly the femoral stem. Because ultrasound signals generally may not penetrate the first bone (i.e., bone surface) that they encounter, internal features of bones, such as internal bone canals (such as the medullary cavity of the femur), may not be included as part of the point clouds 506, 508 obtained using ultrasound imaging. Similarly, some other tissues, such as cartilage, tendons, and/or ligaments, may not be visible using ultrasound because such tissues may be occluded, such as by bone.

Referring to FIG. 30B, in the illustrated embodiment, the preliminary 3-D models 504A, 504B may include the portions of the bones 510, 512 that are not included in the point clouds 506, 508. As discussed in detail above, the preliminary 3-D models 504A, 504B may be created by customizing generalized bone models using the point clouds 506, 508. Generalized bone models may be obtained from a database or statistical atlas including bone information. In the case of the portions of bone or soft tissues missing from the point clouds 506, 508, in some embodiments, the corresponding portions of the preliminary 3-D models 504A, 504B may be generated using the generalized bone models as customized by the available points in the point clouds 506, 508. Thus, while the portions of the preliminary 3-D models corresponding to the portions of the bones 510, 512 that are not included in the point clouds 506 may be customized by the available points in the point clouds 506, 508, those portions of the preliminary 3-D models 504A, 504B may not reflect all patient-specific anatomical deviations from the generalized 3-D model. Specifically, in the illustrated embodiment, the femoral head geometry and/or the acetabular cup geometry may be based predominantly on the generalized 3-D models because patient-specific information about these portions may be unavailable using ultrasound imaging.

In other embodiments, some portions of the relevant anatomy may not be included in the generalized 3-D models. For example, some generalized 3-D bone models may not include some features, such as the medullary cavities.

Referring back to FIG. 28, the method 500 may include an operation 514, including obtaining one or more supplemental images 516 of the relevant anatomy. In the illustrated embodiment, the supplemental image(s) 516 comprise(s) a digital two-dimensional, static X-ray of the one or more anatomical aspects of interest, such as bone and soft tissues. In other embodiments, supplemental image(s) 516 may include one or more scanned X-rays or one or more 2-D static images obtained from fluoroscopy, as well as one or more 2-D and/or 3-D images obtained via other imaging modalities. The supplemental image(s) 516 may comprise partial X-rays and/or a plurality of X-rays, for example. In various example embodiments, the supplemental images 516 may be obtained in connection with general routine office imaging using general routine office imaging equipment and/or may be obtained specifically for use in connection with 3-D model generation as described herein. Generally, in some example embodiments, the point clouds 506, 508 may be obtained utilizing a first imaging modality (e.g., ultrasound) and the supplemental image(s) 516 may be obtained utilizing a second, different imaging modality (e.g., 2-D X-ray, CT, MRI, or fluoroscopy).

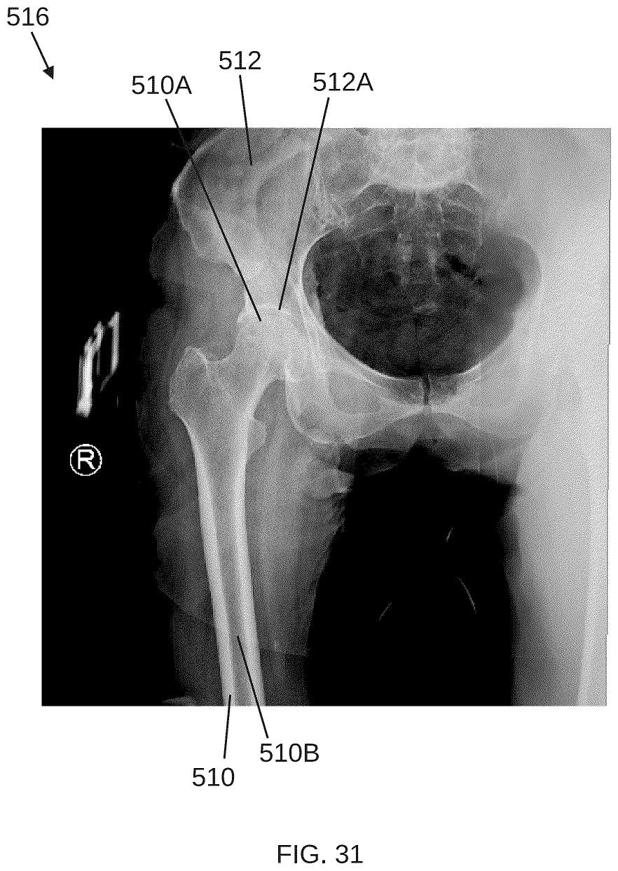

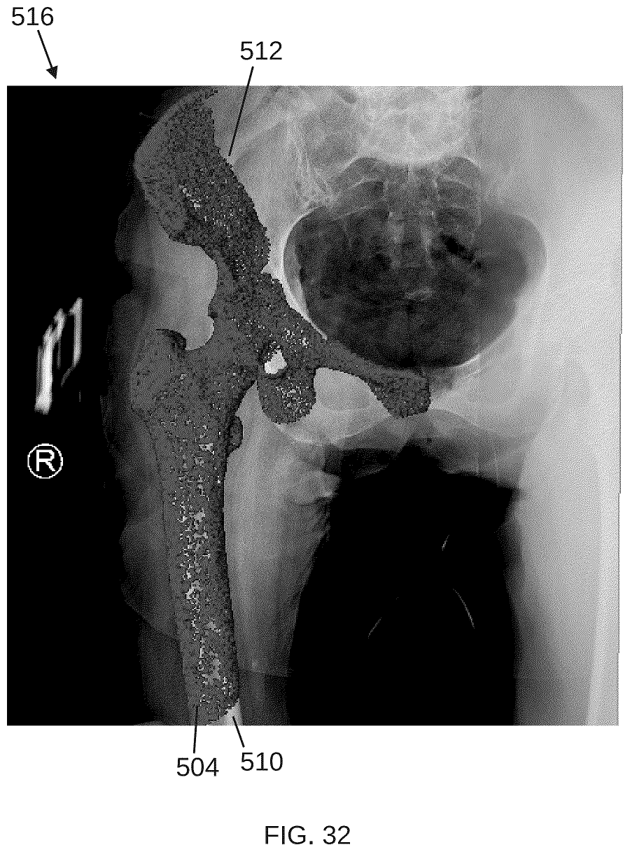

FIG. 31 is an example supplemental image 516 comprising a 2-D X-ray, according to at least some aspects of the present disclosure.

Referring to FIGS. 29A, 29B, and 31, in the illustrated embodiment, the supplemental image 516 depicts at least a portion of the femur 510 and at least a portion of the pelvis 512. Accordingly, in the illustrated embodiment, this supplemental image 516 may be utilized (e.g., as a supplemental image) in connection with generating a virtual 3-D patient-specific anatomic bone model for each of the femur 510 and the pelvis 512. That is, one supplemental image may be utilized in connection with models of more than one anatomical structure of interest. In other embodiments, separate supplemental images may be obtained for various anatomical structures being modeled. Also, in some example embodiments, two or more supplemental images may be obtained and/or utilized in connection with modeling a particular anatomical structure. For example, more than one 2-D X-ray views may be utilized, such for collecting information that may be obtained from one or more views and not other views, or such as may be confirmed or validated using additional views.

In the illustrated embodiment, the supplemental image 516 includes at least some portions of the pertinent anatomy that were not included in the respective point clouds 506, 508. For example, in the illustrated embodiment, the supplemental image 516 depicts the size and/or shape of the femoral head 510A, the size and/or shape of the acetabular cup 512A, and/or the size and/or shape of the intermedullary canal 510B of the femur 510. More generally, in the illustrated embodiment, the supplemental image 516 includes data pertaining to at least one portion of the anatomy that was not included in the imaging (e.g., ultrasound imaging) used to generate the preliminary 3-D model 504. For example, supplemental images 516 comprising X-rays may clearly reveal internal structures of bones.

Referring back to FIG. 28, the method 500 may include an operation 518, including registering the preliminary virtual 3-D model 504 and the supplemental image 516. More specifically, in the illustrated embodiment, the preliminary virtual 3-D model of the femur 504A may be registered with the portion of the supplemental image 516 depicting the femur 510 and/or the preliminary virtual 3-D model of the pelvis 504B may be registered with the portion of the supplemental image 516 depicting the pelvis 512.

FIG. 32 illustrates the preliminary 3-D model 504 (shown as surface points) registered with the supplemental image 516, according to at least some aspects of the present disclosure.

Prior to or concurrent with performing 2-D-3-D registration 518, the exemplary process 500 may include image distortion correction. Specifically, using the 3D preliminary models output from the initial ultrasound imaging, the 2-D images from the additional imaging modality (e.g., X-ray) may be processed by an image distortion correction algorithm that calculates an image magnification factor and the anatomical feature orientation in 3-D space relative to the imaging modality images. Specifically, the image distortion correction algorithm registers the 3-D bone models with the 2-D images to determine the optimal magnification factor and anatomy position in 3-D relative to the 2-D images. The comparison between the 3-D model and the 2-D images is carried out for all or a plurality of the 2-D images, which allows the algorithm to register the 2-D images in space and extract the surface contours in areas where ultrasound data couldn't be collected. Examples where ultrasound data might not be available include, without limitation, the femoral head, the femoral intramedullary canal, and the acetabular cup.

Referring to FIGS. 28 and 32, in the illustrated embodiment, the registration operation 518 may include solving for a pose (e.g., relative position and/or orientation) and/or relative scale/magnification of the preliminary virtual 3-D model 504 that will produce a 2-D projection corresponding to the projection of the supplemental image 516. In some example embodiments, anatomical features visible on both the preliminary virtual 3-D model 504 and the supplemental image 516 may be utilized in the registration operation. Various methods can be utilized in registration, such as optimization. In some example embodiments, registration may be performed individually for anatomical structures of interest, such as the femur 510 and/or the pelvis 512.

In some example embodiments, it may not be necessary to separately determine the scale and/or magnification of the supplemental image 516 (e.g., X-ray image). Specifically, because the sizes of the anatomical structures may be determined from the ultrasound point clouds 506, 508, the scale/magnification of the supplemental image 516 may be determined in connection with the registration operation 518.

Referring back to FIG. 28, the method 500 may include an operation 520 including extracting geometric information from the supplemental image 516. Geometric information extracted from the supplemental image 516 may include, for example and without limitation, one or more length dimensions, angular dimensions, curvatures, etc. pertaining to the relevant bone or soft tissue.

Referring to FIGS. 28 and 31, in the illustrated embodiment, the extracted geometric information may pertain to, for example and without limitation, the size and/or shape of the femoral head 510A, the size and/or shape of the intermedullary canal of the femur 510B, the size and/or shape of the acetabular cup 512A, the thickness of cartilage in the acetabulum, and/or the location of the femoral head ligament. Thus, in this embodiment, at least some of the extracted geometric information may pertain to portions of the bones 510, 512 that were not included in the respective point clouds 506, 508. More generally, in some example embodiments, the supplemental image 516 may provide patient-specific data pertaining to at least one portion of the anatomy that was not included in the imaging used to generate the preliminary virtual 3-D models (e.g., the point clouds 506, 508 obtained using ultrasound imaging). Similarly, in some example embodiments, the supplemental image 516 may provide patient-specific information about at least one portion of the anatomy for which the preliminary virtual 3-D model was based predominantly on a generalized 3-D model. In some embodiments, the extracted geometric information may include one or more parameters that may be directly measured or otherwise directly obtained from the supplemental image 516, such as the size and/or shape of a bone feature. In some embodiments, the extracted geometric information may be utilized to predict and/or estimate a parameter that was not directly measured or otherwise directly obtained from the supplemental image 516. For example, the thickness of the cartilage in the acetabulum (e.g., hip articular cartilage) may be predicted and/or estimated using data from the ultrasound imaging and/or the extracted geometric information from a supplemental image 516 (e.g., X-ray), such as by using a statistical approach to estimate the cartilage based on bone information. In other embodiments, articular cartilage in other joints may be estimated (e.g., knee articular cartilage, shoulder articular cartilage), for example.

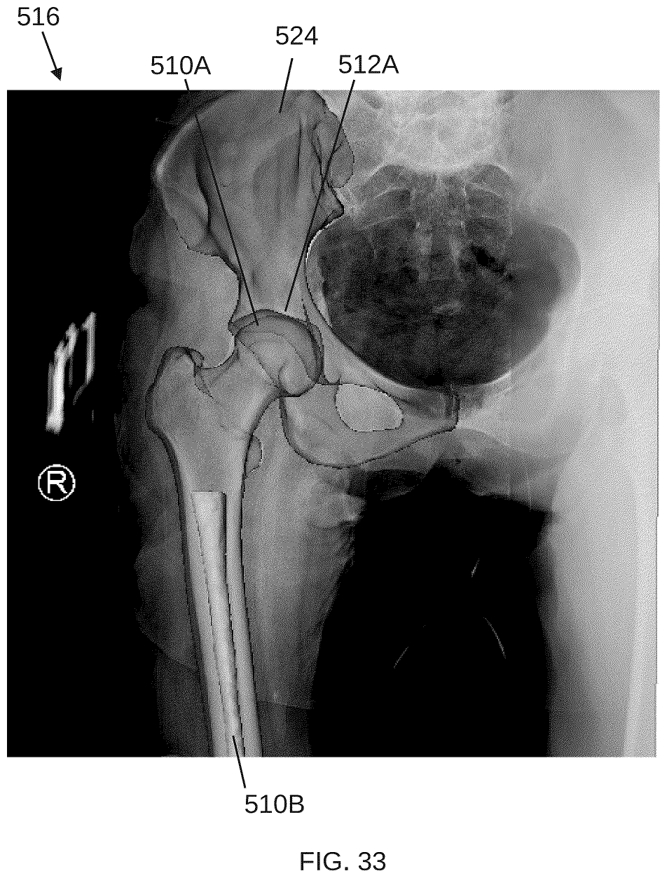

Referring back to FIG. 28, the method 500 may include an operation 522, including generating a refined virtual 3-D patient specific bone model 524 by refining the preliminary virtual 3-D model 504 using at least some of the geometric information extracted from the supplemental image 516. FIG. 33 illustrates the refined virtual 3-D model 524 produced by operation 522 overlain with the supplemental image 516, according to at least some aspects of the present disclosure. A fusion step is then performed to fuse the data extracted from the 2-D images with the 3D ultrasound point cloud (or 3D model), followed by a morphing step to create a new 3D model that captures more accurately the information from both the preliminary 3-D model and the 2-D images.

Referring to FIGS. 28 and 33, because the preliminary 3-D model 504 and the supplemental image 516 were registered in operation 518, the geometric information extracted from the supplemental image 516 in operation 520 can be correlated with the preliminary 3-D model 504. In the illustrated embodiment, the extracted geometric information pertaining to the size and/or shape of the femoral head 510A and/or the acetabular cup 512A may be utilized to refine respective portions of the preliminary 3-D model 504. Specifically, in the illustrated embodiment, where the preliminary 3-D model 504 was based predominantly on the generalized 3-D bone model because the point clouds 506, 508 did not include these portions and at least some of the geometric information extracted from the supplemental image 516 pertains to these portions, the refined 3-D model 524 produced by the refining operation 522 may provide a more accurate, patient-specific representation of these portions of the anatomy than the preliminary 3-D model.

Additionally, in some example embodiments in which statistical atlas generalized 3-D bone models may not include some features, the geometric data extracted from the supplemental image 516 may be used to add such features to the preliminary 3-D model 504. For example, in the illustrated embodiment, the generalized 3-D bone model may not include the intermedullary canal of the femur 510B. Accordingly, the preliminary 3-D model may not include the intermedullary canal 510B. The intermedullary canal 510B may be visible on the supplemental image 516, and relevant geometric information pertaining to the intermedullary canal 510B may be extracted from the supplemental image 516 in operation 520. In the illustrated embodiment, the geometric information pertaining to the intermedullary canal 510B extracted from the supplemental image 516 may be used in the refining operation 522 to add the intermedullary canal 510B feature, thus yielding a refined 3-D model 524 including a patient-specific representation of the intermedullary canal 510B.

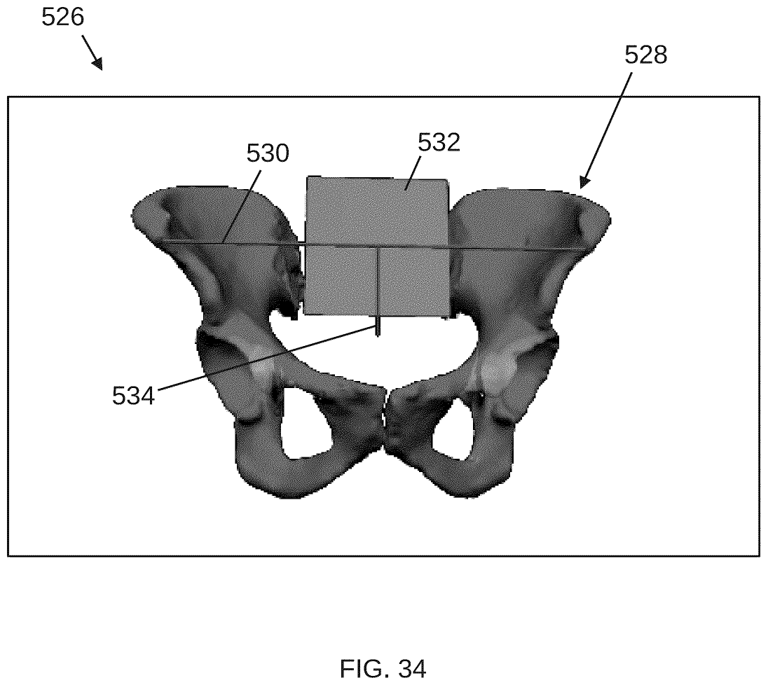

In the illustrated embodiment, the refined 3-D model 524 may include the external topography of the femur and/or the pelvis in the vicinity of the hip joint. In some example embodiments, the refined 3-D model 524 may be used to obtain anatomical measurements of pertinent anatomical features. For example, FIG. 34 illustrates an example display 526 facilitating anatomical measurements using a refined 3-D model 528 of a pelvis. Various locations, dimensions, angles, curvatures, etc., may be determined and/or indicated on the model 528, such as in the form of annotations 530, 532, 534. This information may be used, for example, for preoperative planning, implant design and/or selection (e.g., sizing), etc.

For example, referring to FIG. 34, the refined 3-D model 524 may be used to determine leg length and/or offset, as well as femoral and/or acetabular version, including combined version. As used herein, “femoral version” may refer to the relationship of the axis of the femoral neck to the transcondylar axis of the distal femur. As used herein, “acetabular version” may refer to the angle between a line connecting the anterior acetabular margin with the posterior acetabular margin and perpendicular to a transverse reference line either through the femoral head centers, the posterior acetabular walls, or the respective posterior aspect of the ischial bones. As used herein, “combined version” may refer to the sum of the femoral version and the acetabular version. In some example embodiments, imaging (e.g., ultrasound) may be conducted to provide data pertaining to neck version (e.g., imaging of the femoral neck). In some example embodiments, various images and/or other pertaining to various versions may be provided to a preoperative planner and/or may be used to create a jig. The calculated versions may be used preoperatively for planning and/or intraoperatively, such as to reproduce the neck angle. Similarly, in some example embodiments, imaging (e.g., ultrasound) may be conducted to provide data pertaining to acetabular version and/or cup inclination angles (e.g., imaging of the rim of the acetabulum). The images may be used in a preoperative planner to reproduce the angles with an implanted cup. More generally, various 3-D bone models generated according to at least some aspects of the present disclosure may be used preoperatively, such as to ensure proper version and cup inclination angles.

In some example embodiments, various methods described herein (e.g., method 500) may be performed preoperatively and/or the generated models (e.g., the refined 3-D model 524) may be used preoperatively (e.g., for surgical planning, such as femoral stem sizing, determining cup placement, etc.), intraoperatively (e.g., for surgical navigation), and/or postoperatively (e.g., for postoperative assessment). In some example embodiments, 3-D models generated by example methods described herein may be registered intraoperatively using ultrasound. Unlike intraoperative fluoroscopy, intraoperative use of the 3-D models with ultrasound registration does not expose the patient or nearby personnel (e.g., surgeon) to ionizing radiation. In some example embodiments, preliminary 3-D models 504 may be registered intraoperatively using ultrasound and utilized intraoperatively, without being refined by supplemental images 516.

Additionally, the present disclosure contemplates that in the context of total hip replacements, many total hip arthroplasty procedures may be performed using a posterolateral approach and/or anterolateral approach. Using these approaches typically involves placing the patient in the lateral decubitus position. With the patient positioned laterally as such, radiographic imaging is generally not capable of accurately assessing femoral and/or acetabular version. Intraoperative ultrasound registration of the 3-D models generated according to at least some aspects of the present disclosure may allow accurate determination of femoral version, acetabular version, and/or combined version intraoperatively, such as in real-time or near real-time. More generally, intraoperative ultrasound registration of 3-D models may be useful where patient positioning during surgery is not conducive to registration using other imaging modalities.

It should be understood that while the examples described herein may involve the hip joint and describe 3-D models of the femur and pelvis, it is within the scope of the disclosure that the femur and pelvis be replaced by any one or more anatomical structures (e.g., one or more bones or soft tissues) to achieve similar outcomes. By way of a more detailed example, the shoulder joint may be the subject of this exercise with ultrasound imaging taken of the scapula and proximal humerus. After preliminary patient-specific virtual 3-D bone models are created, these bone models may be further refined using one or more 2-D X-ray images.

Various exemplary embodiments according to at least some aspects of the present disclosure may include apparatus (e.g., ultrasound instrument 50 (FIG. 1)) configured to perform the method 500. Some exemplary embodiments may include a memory (e.g., memory 80 (FIG. 3) or a non-transitory computer readable medium) comprising instructions that, when executed by a processor (e.g., CPU 78 (FIG. 3)), cause the processor to perform the method 500.

Determination of Spine-Pelvis Tilt

The present disclosure contemplates that preoperative planning for some surgeries may include functional assessments and planning. For example, in preoperative planning for hip replacement surgeries, it may be useful to consider the patient's spine-pelvis tilt at one or more functional positions.

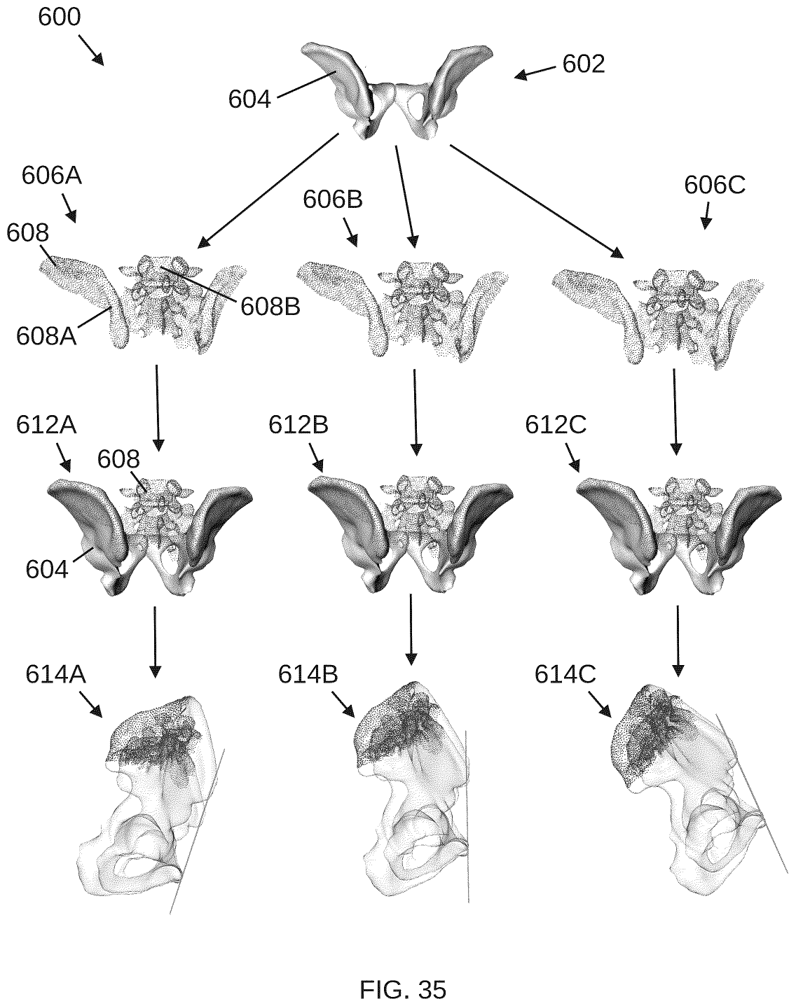

FIG. 35 is a flow diagram illustrating an example method 600 of determining spine-pelvis tilt, according to at least some aspects of the present disclosure. The method 600 may include an operation 602, which may include obtaining a virtual 3-D model 604 of the patient's pelvis. In the illustrated embodiment, this operation 602 may include generating a 3-D model of a patient's pelvis using ultrasound, as described elsewhere herein.

The method 600 may include an operation 606A, which may include obtaining one or more ultrasound point clouds 608 of the patient's pelvis and/or spine, in a first of a series of functional positions. In the illustrated embodiment, the operation 606A may include obtaining a 3-D ultrasound point cloud of the pelvis 608A and/or an ultrasound point cloud of at least a portion of the spine 608B (e.g., lumbar and/or sacrum) in a first functional position (e.g., sitting). In some example embodiments, the point cloud 608 may be generally sparse. In some example embodiments, the point cloud 608 may include additional points pertaining to the patient's femur, which may facilitate determination of the femoral version, the acetabular version, and/or the combined version. For example, the point cloud 608 may include data sufficient to identify the transepicondylar and/or posterior condylar axis of the femur to determine the femoral version angle reference axis. Further, in some example embodiments, information pertaining to leg length may be obtained. For example, data from at least one X-ray taken with the patient in a standing position may be obtained.

The method 600 may include an operation 612A, which may include registering at least a portion of the point cloud 608 with the 3-D model 604 of the pelvis. In the illustrated embodiment, the ultrasound point cloud of the pelvis 608A may be registered with the 3-D model 604 of the pelvis.

The method 600 may include an operation 614A, which may include determining the spine-pelvis tilt in the first functional position using the relative angle of the point cloud of the spine 610A to the 3-D model 604 of the pelvis.

The method 600 may include operations 606B, 612B, and 614B, which may be substantially similar to operations 606A, 612A, and 614A, respectively, except that they are performed with the relevant anatomy in a second functional position (e.g., standing). Similarly, the method 600 may include operations 606C, 612C, and 614C, which may correspond generally to operations 606A, 612A, and 614A, respectively, except that they are performed with the relevant anatomy in a third functional position (e.g., supine).

Some example embodiments may allow 3-D visualization of the spine-pelvis interaction and/or may provide information about the spine-pelvis interaction in each functional position.

Various exemplary embodiments according to at least some aspects of the present disclosure may include apparatus (e.g., ultrasound instrument 50 (FIG. 1)) configured to perform the method 600. Some exemplary embodiments may include a memory (e.g., memory 80 (FIG. 3) or a non-transitory computer readable medium) comprising instructions that, when executed by a processor (e.g., CPU 78 (FIG. 3)), cause the processor to perform the method 600.

3-D Soft Tissue Reconstruction

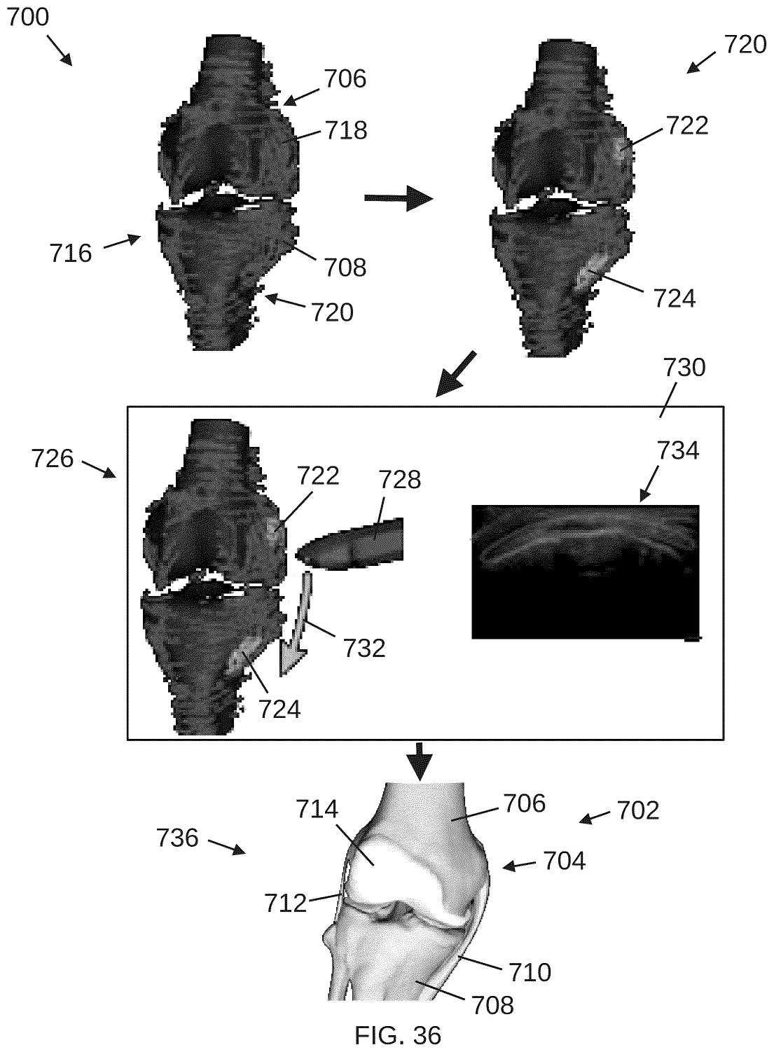

Some exemplary embodiments described herein may focus on generating virtual 3-D models of bones. Some other embodiments according to the present disclosure may include generating virtual 3-D models focusing on and/or including soft tissues, such as ligaments, tendons, cartilage, etc. For example, FIG. 36 is a flow diagram of an example method 700 of generating a virtual 3-D patient-specific model 702 of an anatomical structure (e.g., a knee joint 704 comprising a femur 706 and a tibia 708) including at least one ligament (e.g., a medial collateral ligament 710 and a lateral collateral ligament 712) and/or other soft tissue (e.g., cartilage 714), according to at least some aspects of the present disclosure. It will be understood that various aspects of the method may be utilized in connection with modeling various other anatomical structures, including individual anatomical structures (e.g., individual soft tissues and/or bones), joints comprising a plurality of anatomical structures (hips, knees, shoulders, ankles, etc.).

The method 700 may include an operation 716, including reconstructing a joint (e.g., knee 704) using ultrasound. This operation 716 may be performed generally in the manner described elsewhere herein and/or may include obtaining one or more point clouds 718, 720 associated with bones 706, 708. Operation 716 may produce one or more patient-specific virtual 3-D images and/or models of one or more anatomical structures associated with the joint 702, as described in detail elsewhere herein. For example, the output of operation 716 may comprise one or more patient-specific virtual 3-D bone models.

The method 700 may include an operation 720, including automatically detecting one or more ligament loci (e.g., locations where a ligament attaches to a bone) on the patient-specific virtual 3-D bone model(s). For example, operation 720 may include determining the insertion locations 722, 724 of the medial collateral ligament 710. All ligament loci are pre-defined in a template bone model, which is stored in a .IV format, a special 3D surface representation that maintains model correspondence during processing. The ligament loci are specified by a set of indices stored in the text format. To detect the ligament loci in a patient-specific bone model, the system reconstructs a virtual 3D model of the patient-specific bone that maintains the correspondence to the template bone model. Although the virtual 3D patient-specific bone model and the template bone model may differ, they share some common characteristics of the same bone. By specifying the ligament loci in the template bone model, the system is able to automatically detect the ligament loci in the patient-specific bone model. This innovative approach allows for a more accurate and efficient detection of ligament loci, which is essential for various medical applications.

The method 700 may include an operation 726, including ultrasound scanning of at least a portion of at least one ligament associated with at least one of the detected ligament loci 722, 724. For example, in the illustrated embodiment, the medial collateral ligament 710 may be scanned using an ultrasound probe 728.

In some example embodiments, an ultrasound operator may be provided with automated guidance information for performing the ligament scan. For example, a display 730, which may be shown on an output device such as monitor 58 (FIG. 1) and/or monitor 58′ (FIG. 8), may indicate a current position of the ultrasound probe 728, such as relative to one or more anatomical structures (e.g., bones 706, 708, ligament loci 722, 724, etc.). In some example embodiments, the display 730 may provide specific guidance for conducting the ligament scan, such as an indication (e.g., arrow 732) indicating a desired location and/or direction of scanning. The ultrasound operator may perform the ligament scan using the displayed information. In some example embodiments, the display 730 may include an A-mode or B-mode ultrasound image 734.

The method 700 may include an operation 736, including reconstructing a virtual 3-D model of the soft tissue (e.g., ligament 710). On exemplary method includes using ultrasound. Ultrasound is a dynamic imaging modality meaning, if you fix the transducer in place and move the object being imaged it will capture changes in geometry of that object and its spatial location. With that in mind, the exemplary method may use the reconstructed bone and points thereon, similar to GPS coordinates, to guide the user of the ultrasound transducer to move the transducer to specific locations where soft tissues can be imaged, such as at tendon, muscle, and ligament attachment locations of the bone. At each predetermined location, the user holds the transducer relatively stationary while repositioning the anatomical joint, thus allowing capture of ultrasound data regarding soft tissue locations and changes in cross-sectional area of soft tissue. This information is utilized along with the anatomical joint anatomy and changes in joint flexion angles to construct 3-D soft tissues.

In addition or alternatively, the present method may make use of machine learning to generate 3D models of soft tissues. By way of example, machine learning may include 2-D and/or 3-D data training sets having predetermined features that are identified and associated with specific soft tissues. In exemplary form, as referenced herein, dynamic ultrasound imaging may be utilized in order to image the motion of soft tissues in real-time. By combining machine learning with dynamic ultrasound imaging, the accuracy and efficiency of the 3-D soft tissues constructed is improved.

In some example embodiments, one or more of operations 716, 720, 726, 736 may be repeated one or more times, such as at one or more joint angles across a joint's range of motion. Accordingly, in some example embodiments, a virtual 3-D model of a ligament through a range of motion may be generated.

Various exemplary embodiments according to at least some aspects of the present disclosure may include apparatus (e.g., ultrasound instrument 50 (FIG. 1)) configured to perform the method 700. Some exemplary embodiments may include a memory (e.g., memory 80 (FIG. 3) or a non-transitory computer readable medium) comprising instructions that, when executed by a processor (e.g., CPU 78 (FIG. 3)), cause the processor to perform the method 700.

Guided Diagnostic Scans

Some example embodiments may be configured to provide guidance to an ultrasound operator, which may facilitate improved, more precise, and/or more repeatable ultrasound scans results than relying solely on the skill and/or experience of the ultrasound operators. In some example embodiments, guidance may be provided in an automated manner. For example, arrow 732 in FIG. 36 illustrates example guidance information provided to an ultrasound operator.

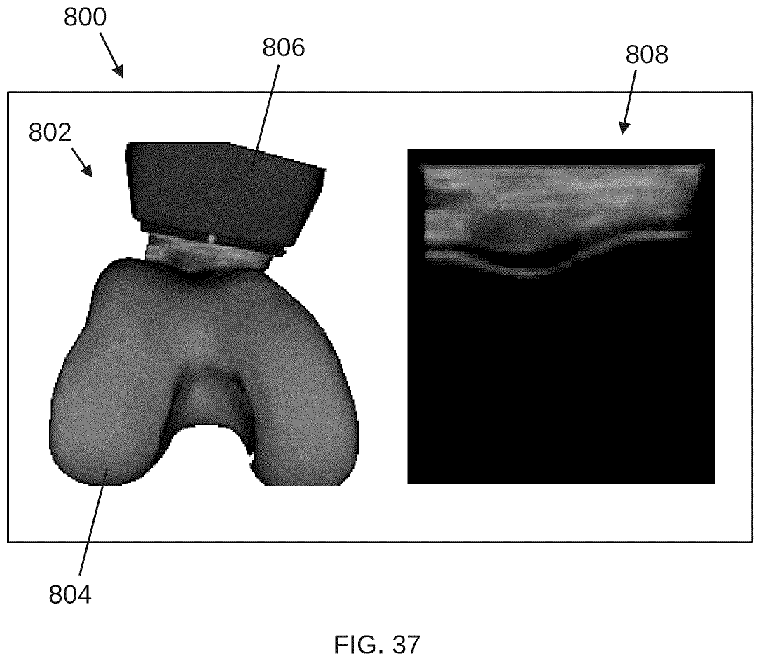

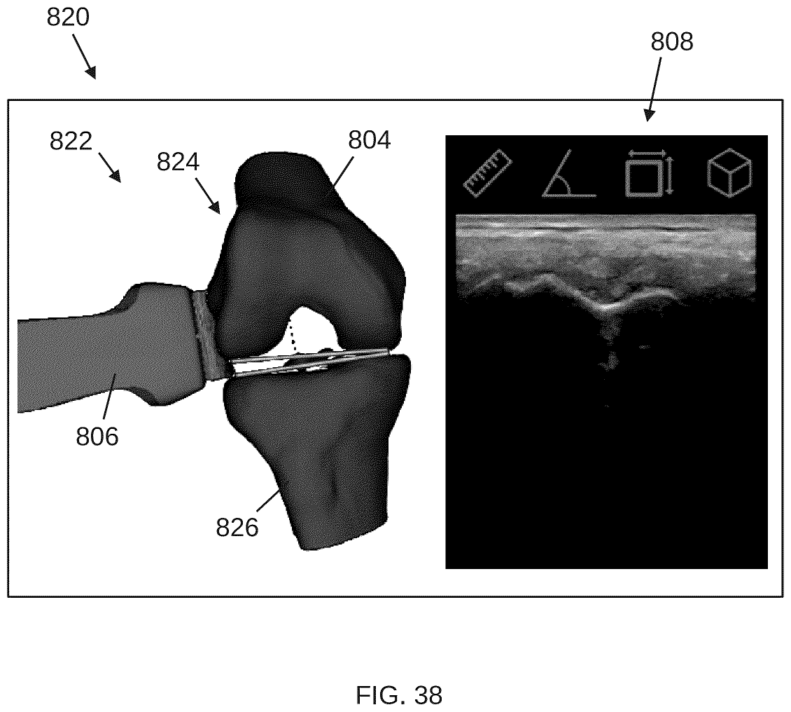

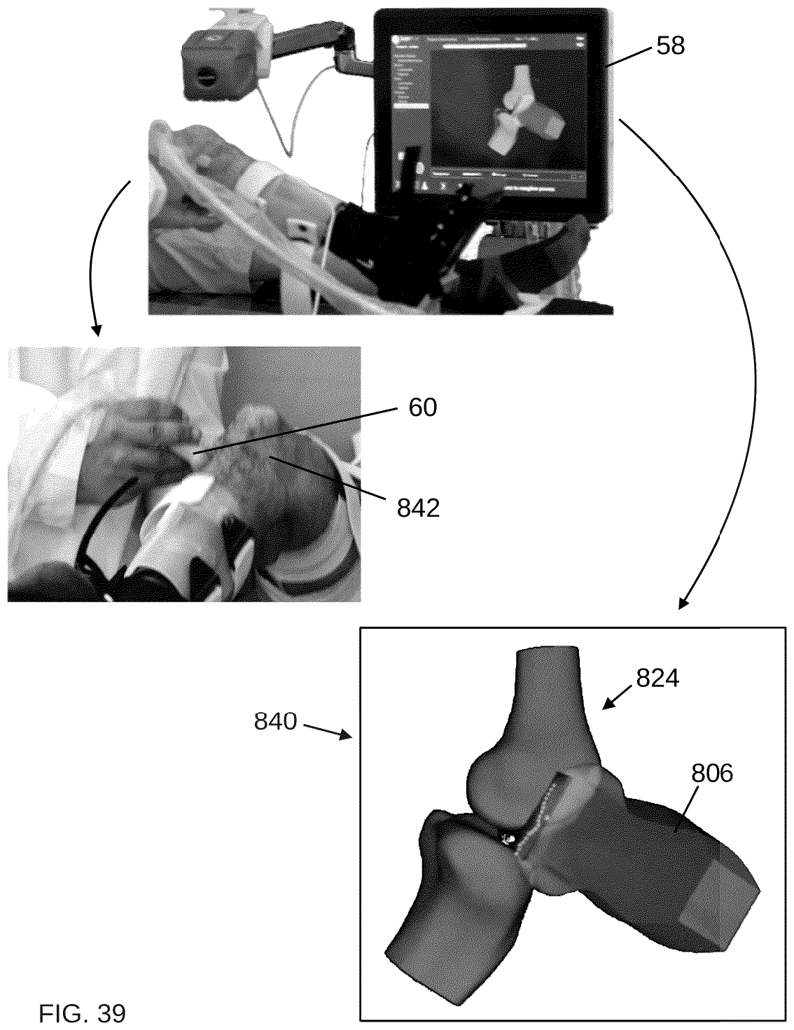

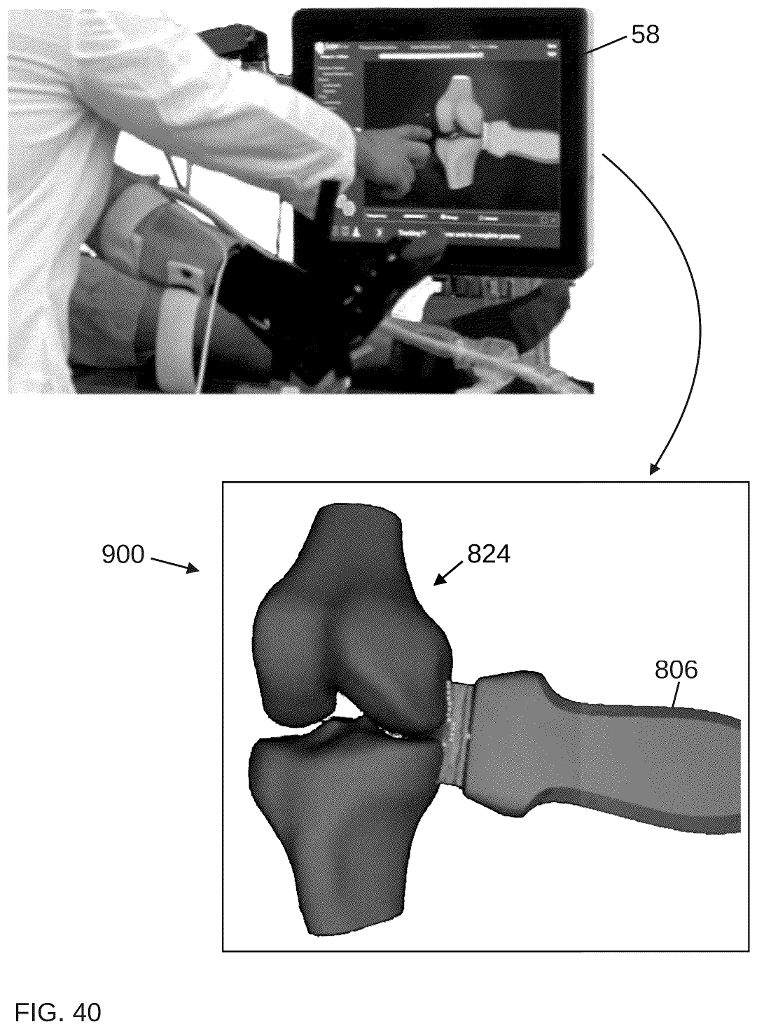

Some example embodiments may be configured to provide one or more displays, such as on monitor 58 (FIG. 1) and/or monitor 58′ (FIG. 8), including information about a current (e.g., periodically and/or constantly updated) position of an ultrasound probe relative to one or more anatomical structures. For example, FIG. 37 is an example display shown during an ultrasound scan of a femur, FIG. 38 is an example display shown during an ultrasound scan of a lateral aspect of a knee, FIG. 39 is an example display shown during an ultrasound scan of a lateral aspect of a knee, and FIG. 40 is an example display shown during an ultrasound scan of a medial aspect of a knee, all according to at least some aspects of the present disclosure.

Referring to FIG. 37, an example display 800 may be shown in connection with an ultrasound scan of a femur. In the illustrated embodiment, the display 800 may include a relative position representation 802, which may include a representation of the femur 804 and/or a representation of the ultrasound probe 806. In the relative position representation 802, the representation of the femur 804 and the representation of the ultrasound probe 806 may be arranged on the display 800 in a manner indicating the current relative positions of the corresponding physical objects. Additionally, in the illustrated embodiment, the display 800 may include an A-mode or B-mode ultrasound image 808 corresponding to the current ultrasound data being obtained by the ultrasound probe.

Referring to FIG. 38, an example display 820 may be shown in connection with an ultrasound scan of a lateral aspect of a knee. In the illustrated embodiment, the display 820 may include relative position representation 822, which may include a representation of the knee 824 (e.g., a representation of the femur 804 and/or a representation of a tibia 826) and/or a representation of the ultrasound probe 806. In the relative position representation 822, the representation of the knee 824 and the representation of the ultrasound probe 806 may be arranged on the display 820 in a manner indicating the current relative positions of the corresponding physical objects. In some example embodiments pertaining to anatomical structures comprising multiple component parts, such as a knee comprising a femur and a tibia, the relative positions of the component parts of the anatomical structure (e.g., representation of the femur 804 and the representation of the tibia 826) relative to each other as well as relative to the representation of the ultrasound probe 806 may be indicated. Additionally, in the illustrated embodiment, the display 820 may include an A-mode or B-mode ultrasound image 808 corresponding to the current ultrasound data being obtained by the ultrasound probe.

Referring to FIG. 39, an example display 840 may be shown in connection with an ultrasound scan of a lateral aspect of a knee. In the illustrated embodiment, the display 840 may be shown on the monitor 58, which may be located near the anatomical structure being imaged (e.g., a patient's knee 842) and the ultrasound probe 60, such as in view of the ultrasound operator. During the imaging/scanning procedure, the relative positions of the representation of the ultrasound probe 806 and the representation of the anatomical structure being imaged (e.g., the representation of the knee 824) may be shown.

Referring to FIG. 40, an example display 900 may be shown during an ultrasound scan of a medial aspect of a knee. In the illustrated embodiment, the display 900 may be shown on the monitor 58, which may be located near the anatomical structure being imaged (e.g., a patient's knee 842) and the ultrasound probe, such as in view of the ultrasound operator. During the imaging/scanning procedure, the relative positions of the representation of the ultrasound probe 806 and the representation of the anatomical structure being imaged (e.g., the representation of the knee 824) may in shown.

Bone Registration System for Intra-Operative Surgery

Intra-operative surgical procedures that involve bone manipulation benefit from accurate registration of the patient's bone or tissue model to the patient's actual anatomy. Precise alignment of the pre-operative 3D patient-specific anatomical model with the intra-operative patient anatomy can help in reducing surgical complications and improving the overall surgical outcome. However, current registration techniques have limitations in achieving accurate alignment, especially in cases where the patient's tissues (including bone and soft tissues) have undergone deformities or changes. Most existing anatomical registration methods are performed after making an incision, leading to blood loss and other surgical complications. Therefore, there is a need for a reliable, accurate, non-invasive, and bloodless anatomical registration system for intra-operative surgery that can overcome the limitations of the existing techniques.

The present disclosure provides an anatomical registration system that enables accurate registration of a pre-operative 3D patient-specific anatomical model to the intra-operative patient anatomy. The anatomical registration system comprises an ultrasound probe, a computer algorithm, and a point cloud registration module. The system uses a combination of anatomical landmarks and ultrasound scans to achieve accurate alignment of the pre-operative 3D patient-specific anatomical model with the intra-operative patient anatomy.

In exemplary form, the anatomy registration system for intra-operative surgery may include an initial registration step, which is preferably performed before making any incision is made intra-operatively. This initial registration step may include identifying one or more (e.g., two, three, four, or more) predefined anatomical landmarks on a pre-operative 3-D patient specific anatomical model. By way of example, these anatomical landmarks may be readily recognizable features such as, without limitation, the tip of a bone or an attachment point of muscle to a bone. Post identifying one or more anatomical landmarks, an ultrasound probe (including an ultrasound transducer) may be utilized to scan corresponding anatomy of the patient corresponding to each anatomical landmark so that ultrasound data is recorded while tracking the 3-D position of the ultrasound transducer. Using the ultrasound data and position tracking data, an algorithm uses a feature-based method to estimate the position and orientation (pose) of the pre-operative 3D patient-specific anatomical model with respect to the intra-operative patient model generated using ultrasound in the operating room, thereby initially registering the pre-operative 3D patient-specific anatomical model to the patient's intraoperative anatomy.

Post initial registration, the exemplary methods disclosed herein may make use of a refined registration. The refined registration may take place after the pre-operative 3D patient-specific anatomical model is aligned with the intra-operative patient bone, where the ultrasound transducer is repositioned to scan the intra-operative patient's anatomy to generate a 3-D point cloud corresponding to points on one or more surfaces of the patient's anatomy (e.g., bone surface points). The 3-D point cloud may then be registered to the pre-operative 3D patient-specific anatomical model to fine-tune the position of the pre-operative 3D patient-specific anatomical model. In exemplary form, registration of the 3-D point cloud to the 3-D anatomical model may be performed by aligning the point cloud to the pre-operative 3D patient-specific anatomical model using an iterative closest point (ICP) algorithm. The ICP algorithm minimizes the distance between the corresponding points of the pre-operative 3D patient-specific anatomical model and the point cloud, thus achieving accurate alignment.

The exemplary bone registration system and method may provide one or more advantages. By way of example, one advantage is the accurate alignment of the pre-operative 3D patient-specific anatomical model with the intra-operative patient anatomy. Another advantage is improved surgical outcomes and reduced complications. And a further advantage is the ability to register preoperative models with the patient's intraoperative anatomy where the anatomy has undergone significant changes or exhibits material deformities. The exemplary system and methods can be used in any surgical procedure where correlating the virtual realm with the real-world is advantageous.

Non-Invasive Pinless Bone and Spine Tracking System and Method Using Ultrasound and Localization Technology

The present disclosure provides a system and associated non-invasive methods for tracking a patient's anatomy (including bones, such as the pelvis and vertebrae) during surgery using ultrasound and localization technology.

In orthopedic and spine surgeries, computer-assisted surgery is used to track and locate bones and the spine using bone arrays or optical trackers. These trackers are typically mounted to the patient's bone, which may cause longer recovery time and potential complications after surgery. Therefore, there is a need for a non-invasive method to track a patient's anatomy (including bones such as the pelvis and vertebrae) during surgery.

The present disclosure provides pinless bone and spine tracking system and method using a custom-made ultrasound probe. An exemplary ultrasound probe may include an anatomical shape compatible with soft tissue around the target bone or spine. By way of example, the ultrasound probe may be shaped to engage an exterior of the patient anatomy in only a single position and orientation so that signals received by the ultrasound probe during surgical tracking have a fixed frame of reference. Specifically, the bone or spine surface is detected by the ultrasound probe and used to generate a 3-D point cloud representative of surface points on the bone or spine. These surface points are constantly tracked in real time (using one or more electromagnetic (EM) sensors, optical arrays, inertial measurement units, etc.) in order to provide information to a surgeon regarding the current position and orientation of the anatomy.

In exemplary form, an exemplary pinless bone and spine tracking system and methods may make use of a customized ultrasound probe having an anatomical shape compatible with soft tissue around the target bone or spine. The ultrasound probe includes an ultrasound transducer that detects the bone or spine surface by receiving ultrasound echoes and generating data representative of the echoes that is used by the computer system and associated algorithms to generate a 3-D point cloud representation of the bone or spine in a static position. The ultrasound probe may be outfitted with a tracker in order to track the 3-D position and orientation of the ultrasound probe. Exemplary trackers include, without limitation, electromagnetic (EM) sensors, optical arrays, and inertial measurement units. After the 3-D point cloud is generated, and the 3-D point cloud is registered to the patient's anatomy, as discussed above. Post registration, the ultrasound probe may be utilized to track a bone or vertebrae combining motion signals from the ultrasound probe and tissue depth measurements from echoes received by the ultrasound transducer, thereby providing a real-time, non-invasive tracking of anatomy.

By way of example, an exemplary system using the pinless bone and spine tracking system may be used by positioning the ultrasound probe is placed on the skin of the patient proximate the target bone or vertebrae. Thereafter, the ultrasound probe may be repositioned in 3-D, while its 3-D position and orientation are tracked, in order to perform a scan of the patient's anatomy (that is preferably stationary). Those skilled in the art are familiar with ultrasound transducers and scans of a patient's anatomy and, accordingly, a detailed description of this aspect of the method is omitted in furtherance of brevity. As positional and orientation information concerning the ultrasound probe are fed to the computer during the scan, the ultrasound probe is likewise generating signal data indicative of echoes detected by the transducer(s), which allows the computer system to generate 3-D points corresponding to surface points for the anatomy in question, such as one or more bone such as vertebrae. Those skilled in the art will realize that these 3-D points when combined are operative to form a 3-D point cloud, which point cloud is representative of the patient's anatomy or real-world anatomy. Thereafter the system operates to register the point cloud to a patient-specific anatomical model generated pre-operatively (and possibly supplemented intraoperatively as discussed herein) and optionally displays the patient-specific anatomical model on a graphical display accessible to a surgeon. Post registration, position and orientation data from the ultrasound probe are combined with signal data from the transducer(s) by the computer system to generate one or more 3-D points, which are correlated to the patient-specific anatomical model. In this manner, the 3-D points are associated with the patient-specific anatomical model in order to update, in real-time or near real-time, the position and orientation of the patient-specific anatomical model displayed on the display.

Not surprisingly, the exemplary system and methods provide a number of advantages over conventional surgical tracking systems. By way of example, the exemplary surgical tracking system is non-invasive, which lessens the potential complications and reduces patient recovery time compared to invasive surgical trackers. When using a custom shaped ultrasound probe, tracking of the anatomy in question can be simplified because the probe is configured to engage the patient's anatomy in a single position and orientation, thereby providing a fixed frame of reference for changes in 3-D position of the probe, as well as changes in 3-D position of the anatomy in question. Moreover, the exemplary system and methods are not specifically tied to any position and orientation tracking technology and can be used with any position and tracking technology including, without limitation, EM, IMU, and optical trackers.

Generally, apparatus associated with methods described herein may include computers and/or processors configured to perform such methods, as well as software and/or storage devices comprising or storing instructions configured to cause a computer or processor to perform such methods. In some example embodiments, some operations associated with some methods may be performed by two or more computers and/or processors, which may or may not be co-located. For example, some operations and/or methods may be performed by remote computers or servers, and the resulting outputs may be provided to other devices for preoperative, intraoperative, and/or postoperative use.

Although various example embodiments have been described herein in connection with specific anatomies, it will be understood that similar methods and apparatus may be utilized in connection with other anatomies, such as any joint. For example, various embodiments according to at least some aspects of the present disclosure may be used in connection with anatomical structures associated with shoulder joints, hip joints, knee joints, ankle joints, etc.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A method of generating a virtual 3-D patient-specific bone model, the method comprising:
obtaining a preliminary virtual 3-D bone model of a first bone;
obtaining a supplemental image of the first bone;
registering the preliminary virtual 3-D bone model of the first bone with the supplemental image of the first bone;
extracting geometric information about the first bone from the supplemental image of the first bone; and
generating a refined virtual 3-D patient-specific bone model of the first bone by refining the preliminary virtual 3-D bone model of the first bone using the geometric information about the first bone from the supplemental image of the first bone.

2. The method of claim 1, wherein obtaining the preliminary 3-D bone model comprises obtaining a point cloud of the first bone and reconstructing the preliminary 3-D bone model by morphing a generalized 3-D bone model using the point cloud of the first bone.

3. The method of claim 2,
wherein obtaining the point cloud of the first bone utilizes a first imaging modality;
wherein obtaining the supplemental image of the first bone utilizes a second imaging modality; and
wherein the first imaging modality is different than the second imaging modality.

4. The method of claim 3, wherein the first imaging modality comprises ultrasound.

5.-22. (canceled)

23. The method of claim 1, wherein registering the preliminary 3-D bone model of the first bone with the supplemental image of the first bone comprises solving for a pose of the preliminary 3-D bone model which produces a 2-D projection corresponding to a projection of the supplemental image.

24. The method of claim 1,
wherein obtaining a supplemental image of the first bone comprises obtaining a plurality of supplemental images of the first bone;
wherein registering the preliminary virtual 3-D bone model of the first bone with the supplemental image of the first bone comprises registering the preliminary virtual 3-D bone model of the first bone with the plurality of supplemental images of the first bone;
wherein extracting geometric information about the first bone from the supplemental images of the first bone comprises extracting geometric information about the first bone from the plurality of supplemental images of the first bone; and
wherein refining the preliminary virtual 3-D bone model of the first bone using the geometric information about the first bone from the supplemental image of the first bone comprises refining the preliminary virtual 3-D bone model of the first bone using the geometric information about the first bone from the plurality of supplemental images of the first bone.

25. The method of claim 1, further comprising:
obtaining a preliminary virtual 3-D bone model of a second bone;
obtaining a supplemental image of the second bone;
registering the preliminary virtual 3-D bone model of the second bone with the supplemental image of the second bone;
extracting geometric information about the second bone from the supplemental image of the second bone; and
generating a refined virtual 3-D patient-specific bone model of the second bone by refining the preliminary virtual 3-D bone model of the second bone using the geometric information about the second bone from the supplemental image of the second bone.

26. The method of claim 25,
wherein obtaining the point cloud of the second bone comprises performing an ultrasound scan of the second bone;
wherein obtaining the supplemental image of the second bone comprises obtaining a 2-D X-ray of the second bone; and
wherein the 2-D X-ray of the second bone includes at least one portion of the second bone that was not visible on the ultrasound scan of the second bone.

27. The method of claim 1, wherein extracting geometric information from the supplemental image of the first bone comprises extracting at least one of a length dimension, an angular dimension, or a curvature of the first bone from the supplemental image of the first bone.

28.-30. (canceled)

31. A method of generating a virtual 3-D patient-specific bone model, the method comprising:
obtaining ultrasound data pertaining to an exterior surface of a first bone;
obtaining X-ray data pertaining to at least one of an internal feature of the first bone and/or an occluded feature of the first bone; and
generating a 3-D patient-specific bone model of the first bone using the ultrasound data and the X-ray data, the 3-D patient-specific bone model representing the exterior surface of the first bone and the at least one of the internal feature of the first bone and/or the occluded feature of the first bone.

32.-34. (canceled)

35. A method of determining a spine-pelvis tilt, the method comprising:
obtaining a virtual 3-D model of a pelvis;
obtaining a first ultrasound point cloud of the pelvis and a first ultrasound point cloud of a lumbar spine with the pelvis and the lumbar spine in a first functional position;
registering the virtual 3-D model of the pelvis to the first point cloud of the pelvis; and
determining a first spine-pelvis tilt in the first functional position using a first relative angle of the first point cloud of the lumbar spine to the 3-D model of the pelvis.

36. The method of claim 35, further comprising positioning at least one of the pelvis or the lumbar spine into the first functional position.

37. The method of claim 35, further comprising
obtaining a second ultrasound point cloud of the pelvis and a second ultrasound point cloud of the lumbar spine with the pelvis and the spine in a second functional position;
registering the virtual 3-D model of the pelvis to the second point cloud of the pelvis; and
determining a second spine-pelvis tilt in the second functional position using a second relative angle of the second point cloud of the lumbar spine to the 3-D model of the pelvis.

38. The method of claim 37, further comprising positioning at least one of the pelvis or the lumbar spine into the second functional position.

39. The method of claim 37, further comprising
obtaining a third ultrasound point cloud of the pelvis and a third ultrasound point cloud of the lumbar spine with the pelvis and the lumbar spine in a third functional position;
registering the virtual 3-D model of the pelvis to the third point cloud of the pelvis; and
determining a third spine-pelvis tilt in the third functional position using a third relative angle of the third point cloud of the lumbar spine to the 3-D model of the pelvis.

40.-48. (canceled)

49. A method of generating a virtual 3-D patient-specific model of a ligament, the method comprising:
obtaining a virtual 3-D patient-specific bone model of a joint;
detecting at least one ligament loci on the virtual 3-D patient-specific bone model;
obtaining ultrasound data pertaining to a ligament associated with the at least one ligament loci by scanning, using ultrasound, the ligament; and
reconstructing a virtual 3-D model of the ligament using the ultrasound data.

50. The method of claim 49, wherein obtaining the ultrasound data pertaining to the ligament is performed at a plurality of joint angles of the joint across the joint's range of motion.

51. The method of claim 49, wherein obtaining the virtual 3-D patient-specific bone model of the joint comprises reconstructing the joint using ultrasound.

52. The method of claim 51, wherein reconstructing the joint using ultrasound comprises obtaining at least one point cloud associated with one or more bones of the joint.

53. The method of claim 49, wherein detecting the at least one ligament loci on the patient-specific virtual 3-D bone model comprises determining at least one insertion location of the ligament.

54. The method of claim 49, wherein scanning, using ultrasound, the ligament comprises providing automated guidance information.

55. The method of claim 54, wherein providing the automated guidance information comprises providing a display comprising a current position of an ultrasound probe relative to one or more anatomical structures.

56. The method of claim 54, wherein providing the automated guidance information comprises providing a display comprising an indication of a desired location or direction of scanning.

57. The method of claim 54, wherein providing the automated guidance information comprises providing a display comprising an A-mode or B-mode ultrasound image.

58.-61. (canceled)

62. A method of generating a virtual 3-D patient-specific anatomical model, the method comprising:
obtaining a preliminary virtual 3-D anatomy model of a first anatomy;
obtaining a supplemental image of the first anatomy;
registering the preliminary virtual 3-D anatomy model of the first anatomy with the supplemental image of the first anatomy;
extracting geometric information about the first anatomy from the supplemental image of the first anatomy; and
generating a refined virtual 3-D patient-specific anatomy model of the first anatomy by refining the preliminary virtual 3-D anatomy model of the first anatomy using the geometric information about the first anatomy from the supplemental image of the first anatomy.

63. The method of claim 62, wherein obtaining the preliminary 3-D anatomy model comprises obtaining a point cloud of the first anatomy and reconstructing the preliminary 3-D anatomy model by morphing a generalized 3-D anatomy model using the point cloud of the first anatomy.

64. The method of claim 63,
wherein obtaining the point cloud of the first anatomy utilizes a first imaging modality;
wherein obtaining the supplemental image of the first anatomy utilizes a second imaging modality; and
wherein the first imaging modality is different than the second imaging modality.

65.-83. (canceled)

84. The method of claim 62, wherein registering the preliminary 3-D anatomy model of the first anatomy with the supplemental image of the first anatomy comprises solving for a pose of the preliminary 3-D anatomy model which produces a 2-D projection corresponding to a projection of the supplemental image.

85. The method of claim 62,
wherein obtaining a supplemental image of the first anatomy comprises obtaining a plurality of supplemental images of the first anatomy;
wherein registering the preliminary virtual 3-D anatomy model of the first anatomy with the supplemental image of the first anatomy comprises registering the preliminary virtual 3-D anatomy model of the first anatomy with the plurality of supplemental images of the first anatomy;
wherein extracting geometric information about the first anatomy from the supplemental images of the first anatomy comprises extracting geometric information about the first anatomy from the plurality of supplemental images of the first anatomy; and
wherein refining the preliminary virtual 3-D anatomy model of the first anatomy using the geometric information about the first anatomy from the supplemental image of the first anatomy comprises refining the preliminary virtual 3-D anatomy model of the first anatomy using the geometric information about the first anatomy from the plurality of supplemental images of the first anatomy.

86. The method of claim 62, further comprising:
obtaining a preliminary virtual 3-D anatomy model of a second anatomy;
obtaining a supplemental image of the second anatomy;
registering the preliminary virtual 3-D anatomy model of the second anatomy with the supplemental image of the second anatomy;
extracting geometric information about the second anatomy from the supplemental image of the second anatomy; and
generating a refined virtual 3-D patient-specific anatomy model of the second anatomy by refining the preliminary virtual 3-D anatomy model of the second anatomy using the geometric information about the second anatomy from the supplemental image of the second anatomy.

87. The method of claim 86,
wherein obtaining the point cloud of the second anatomy comprises performing an ultrasound scan of the second anatomy;
wherein obtaining the supplemental image of the second anatomy comprises obtaining a 2-D X-ray of the second anatomy; and
wherein the 2-D X-ray of the second anatomy includes at least one portion of the second anatomy that was not visible on the ultrasound scan of the second anatomy.

88. The method of claim 62, wherein extracting geometric information from the supplemental image of the first anatomy comprises extracting at least one of a length dimension, an angular dimension, or a curvature of the first anatomy from the supplemental image of the first anatomy.

89.-94. (canceled)
